SEQUENCE LISTING (1 sequence)

SEQ ID NO: 1 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

(19) United States
(12) Patent Application Publication
(10) Pub. No.: US 2024/0316062 A1
(43) Pub. Date: Sep. 26, 2024

(54) COMBINATION OF IMMUNOTHERAPEUTICS AND BISFLUOROALKYL-1,4-BENZODIAZEPINONE COMPOUNDS FOR TREATING CANCER

(71) Applicant: Immunome, Inc., Bothell, WA (US)

(72) Inventors: David SIDRANSKY, Pikesville, MD (US); Amnon PELED, Tel-Aviv (IL)

(73) Assignee: Immunome, Inc., Bothell, WA (US)

(21) Appl. No.: 18/738,096

(22) Filed: Jun. 10, 2024

Related U.S. Application Data
(63) Continuation of application No. 17/746,205, filed on May 17, 2022, now abandoned.
Application No. 17/746,205 is a continuation of application No. 16/580,178, filed on Sep. 24, 2019, now abandoned.
Application No. 16/580,178 is a continuation-in-part of application No. PCT/US2019/030996, filed on May 7, 2019.
(60) Provisional application No. 62/787,406, filed on Jan. 2, 2019.
(60) Provisional application No. 62/715,293, filed on Aug. 7, 2018.
(60) Provisional application No. 62/667,644, filed on May 28, 2018.
(60) Provisional application No. 62/675,787, filed on May 24, 2018.

(51) Int. Cl. A61K 31/5513; A61K 9/00; A61K 35/17; A61K 38/17; A61K 39/395; A61P 35/00

(52) U.S. Cl. CPC A61K 31/5513; A61K 9/0019; A61K 9/0053; A61K 35/17; A61K 38/1774; A61K 39/3955; A61P 35/00

(57) ABSTRACT

The present invention provides methods of use for compositions comprising an immunotherapeutic such as chimeric antigen receptor T cells (CAR-T cells), and specifically those that target a tumor antigen cleaved by gamma secretase, in combination with bisfluoroalkyl-1,4-benzodiazepinone compounds, including compounds of Formula (I) or prodrugs thereof;

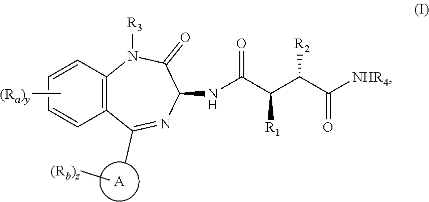

for treating lymphomas.

### CROSS REFERENCE TO RELATED APPLICATIONS

This application is a Continuation of U.S. patent application Ser. No. 17/746,205, filed on May 17, 2022, which is a Continuation of U.S. patent application Ser. No. 16/580,178 filed on Sep. 24, 2019, which is a Continuation-in-Part of PCT International Application No. PCT/US2019/030996, filed on May 7, 2019, which claims the benefit of U.S. Provisional Patent Application No. 62/787,406 filed on Jan. 2, 2019, U.S. Provisional Patent Application No. 62/715,293 filed on Aug. 7, 2018, U.S. Provisional Patent Application No. 62/667,644 filed on May 28, 2018, and U.S. Provisional Patent Application No. 62/675,787 filed on May 24, 2018, which are incorporated in their entirety herein by reference.

### SEQUENCE LISTING STATEMENT

The instant application contains a Sequence Listing which has been submitted electronically in XML file format and is hereby incorporated by reference in its entirety. Said XML copy, created on Jun. 9, 2024, is named P-579617-US2-SQL-ST26-09JUN24 and is 3 KB in size.

### FIELD OF THE INVENTION

The present invention provides methods of use for compositions comprising an immunotherapeutic such as chimeric antigen receptor T cells (CAR-T cells), and specifically those that target a tumor antigen cleaved by gamma secretase, in combination with bisfluoroalkyl-1,4-benzodiazepinone compounds, including compounds of Formula (I) or prodrugs thereof;

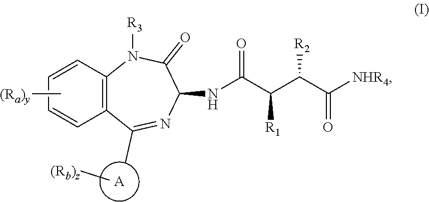

for treating lymphomas.

### BACKGROUND OF THE INVENTION

The present invention provides a method of improving the efficacy of a BCMA-targeting immuno-therapeutic in a subject having lymphoma comprising the step of administering to said subject a first composition comprising one or more compounds represented by the structure of Formula (I):

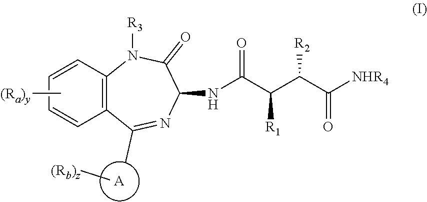

and/or at least one salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

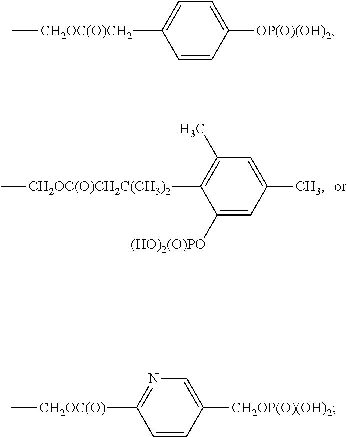

R_{y} is: —SCH₂CH(NH₂)C(O)OH, —SCH₂CH(NH₂)C(O)OCH₃, or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is 1 or 2,
   and a second composition comprising one or more BCMA-targeting immuno-therapeutics.

The present invention also provides a method of treating, suppressing or inhibiting a lymphoma in a subject comprising the step of administering to said subject a first composition comprising one or more compounds represented by the structure of Formula (I):

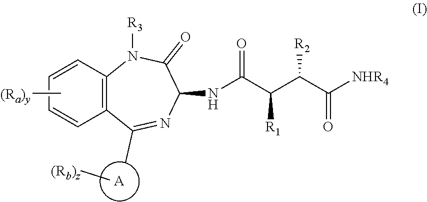

and/or at least one salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

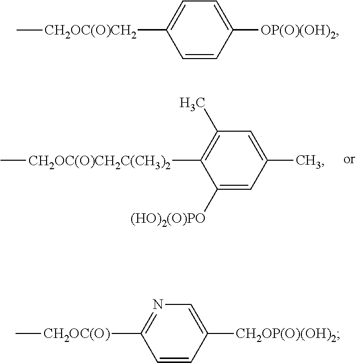

R_{y} is: —SCH₂CH(NH₂)C(O)OH, —SCH₂CH(NH₂)C(O)OCH₃, or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is 1 or 2,
   and a second composition comprising one or more BCMA-targeting immuno-therapeutics.

The present invention also provides a method of decreasing B-cell maturation antigen (BCMA) shedding or decreasing soluble BCMA in a lymphoma cell in a subject having lymphoma comprising the step of administering to said subject a composition comprising one or more compounds represented by the structure of Formula (I):

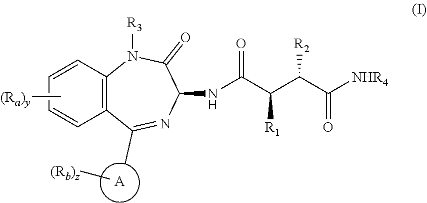

and/or at least one salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

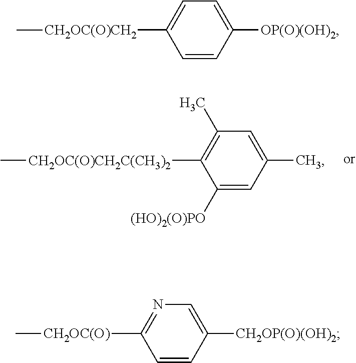

R_{y} is: —SCH₂CH(NH₂)C(O)OH, —SCH₂CH(NH₂)C(O)OCH₃, or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is 1 or 2.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1. Levels of cell-bound BCMA on multiple myeloma (MM) cells incubated with γ-secretase inhibitors (GSIs). U266 cells (MM cell line) were incubated with 0.3 nM, 0.5 nM, 1 nM or 3 nM of GSI (Compound 1, Compound 22, LY3039478 or PF3084014) for 24 hours, and BCMA levels were measured by flow cytometry.

FIG. 2A. Levels of soluble BCMA in cell media after incubation of multiple myeloma (MM) cells with Compound 22. U266 cells (MM cell line) were incubated with 0.3 nM, 1 nM, 3 nM and 10 nM of Compound 22 for 24 hours, and levels of soluble BCMA were measured by ELISA.

FIG. 2B. Levels of soluble BCMA in cell media after incubation of multiple myeloma (MM) cells with LY3039478. U266 cells (MM cell line) were incubated with 0.3 nM, 1 nM, 3 nM and 10 nM of LY3039478 for 24 hours, and levels of soluble BCMA were measured by ELISA.

FIG. 2C. Levels of soluble BCMA in cell media after incubation of multiple myeloma (MM) cells with Compound 1. U266 cells (MM cell line) were incubated with 0.3 nM, 1 nM, 3 nM and 10 nM of Compound 1 for 24 hours, and levels of soluble BCMA were measured by ELISA.

FIG. 2D. Levels of soluble BCMA in cell media after incubation of multiple myeloma (MM) cells with PF3084014. U266 cells (MM cell line) were incubated with 0.3 nM, 1 nM, 3 nM and 10 nM of PF3084014 for 24 hours, and BCMA levels were measured by ELISA.

FIG. 3. Levels of soluble BCMA in cell media after incubation of multiple myeloma (MM) cells with γ-secretase inhibitors (GSIs). U266 cells (MM cell line) were incubated with 0.3 nM, 1 nM, 3 nM and 10 nM of GSI (Compound 1, Compound 22, LY3039478 or PF3084014) for 24 hours, and BCMA levels were measured by ELISA.

FIG. 4A. Levels of BCMA in Lymphoma Cell Lines after treatment with the γ-secretase inhibitor (GSI), Compound 1. Cells from five different lymphoma cell lines (SP49 (MCL with Notch4 GOF); Toledo (DLBCL); RC (Double Hit DLBCL—MYC, BCL2); SUDHL-4 (DLBCL); and SUDHL-6 (DLBCL)) were treated for 24 hours with 0, 1 nM, 10 nM, 100 nM, 500 nM, 1000 nM, or 10000 nM of the GSI Compound 1 (Cell surface BCMA levels were observed by flow cytometry (FACS analysis). * indicates missing data point.

FIG. 4B. Levels of BCMA in Lymphoma Cell Lines after treatment with γ-secretase inhibitor (GSI), Compound 22. Cells from five different lymphoma cell lines (SP49 (MCL with Notch4 GOF); Toledo (DLBCL); RC (Double Hit DLBCL—MYC, BCL2); SUDHL-4 (DLBCL); and SUDHL-6 (DLBCL)) were treated for 24 hours with 0, 1 nM, 10 nM, 100 nM, 500 nM, 1000 nM, or 10000 nM of the GSI, Compound 22. Cell surface BCMA levels were observed by flow cytometry (FACS analysis).

FIG. 5A. Levels of BCMA in B-ALL Cell Line RSV411 after treatment with γ-secretase inhibitors (GSIs). Cells from Acute B-Cell Lymphoblastic Leukemia (B-ALL) cell line RSV411 were treated for 24 hours with different concentrations (0-10 μM) of GSIs (Compound 1 and Compound 22). Cell surface BCMA levels were observed by flow cytometry (FACS analysis).

FIG. 5B. Levels of BCMA in B-ALL Cell Line REH after treatment with γ-secretase inhibitors (GSIs). Cells from Acute B-Cell Lymphoblastic Leukemia (B-ALL) cell line REH were treated for 24 hours with different concentrations (0-10 μM) of GSIs (Compound 1 and Compound 22). Cell surface BCMA levels were observed by flow cytometry (FACS analysis).

FIG. 5C. Levels of BCMA in B-ALL Cell Line 018Z after treatment with γ-secretase inhibitors (GSIs). Cells from Acute B-Cell Lymphoblastic Leukemia (B-ALL) cell line 018Z were treated for 24 hours with different concentrations (0-10 μM) of GSIs (Compound 1 and Compound 22). Cell surface BCMA levels were observed by flow cytometry (FACS analysis).

FIG. 6A. Cell surface BCMA levels in BCP-1 cells (B-cell Lymphoma) after treatment with Compound (22). BCP-1 cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

FIG. 6B. BCMA Shedding in BCP-1 cells (B-cell Lymphoma line) after treatment with Compound (22). BCP-1 cells were incubated with 10 μM, 1 μM, 100 nM, 10 nM, 1 nM, 100 pM, 10 pM, 1 pM, and 100 fM of Compound (22) for 24 hours BCMA levels were measured by ELISA.

FIG. 7A. Cell surface BCMA levels in Daudi cells (Burkitt's Lymphoma) after treatment with Compound (22). Daudi cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

FIG. 7B. BCMA Shedding in Daudi cells (Burkitt's Lymphoma) after treatment with Compound (22). Daudi cells were incubated with 10 μM, 1 μM, 100 nM, 10 nM, 1 nM, 100 pM, 10 pM, 1 pM, and 100 fM of Compound (22) for 24 hours BCMA levels were measured by ELISA.

FIG. 8A. Cell surface BCMA levels in Jiyoye cells (Burkitt's Lymphoma) after treatment with Compound (22). Jiyoye cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

FIG. 8B. BCMA Shedding in Jiyoye cells (Burkitt's Lymphoma) after treatment with Compound (22). Jiyoye cells were incubated with 10 μM, 1 μM, 100 nM, 10 nM, 1 nM, 100 pM, 10 pM, 1 pM, and 100 fM of Compound (22) for 24 hours BCMA levels were measured by ELISA.

FIG. 9A. Cell surface BCMA levels in DB cells (DLBCL) after treatment with Compound (22). DB cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

FIG. 9B. BCMA Shedding in DB cells (DLBCL) after treatment with Compound (22). DB cells were incubated with 10 μM, 1 μM, 100 nM, 10 nM, 1 nM, 100 pM, 10 pM, 1 pM, and 100 fM of Compound (22) for 24 hours BCMA levels were measured by ELISA.

FIG. 10A. Cell surface BCMA levels in RPMI-6666 cells (Hodgkin's Lymphoma) after treatment with Compound (22). RMPI 6666 cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

FIG. 10B. Cell surface BCMA levels in Jeko-1 cells (Mantle Cell Lymphoma) after treatment with Compound (22). Jeko-1 cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

FIG. 10C. Cell surface BCMA levels in JVM-2 cells (Mantle Cell Lymphoma) after treatment with Compound (22). JVM-2 cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

FIG. 10D. Cell surface BCMA levels in DOHH-2 cells (DLBCL) after treatment with Compound (22). DOHH-2 cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

FIG. 10E. Cell surface BCMA levels in HT cells (DLBCL) after treatment with Compound (22). HT cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

FIG. 10F. Cell surface BCMA levels in ST486 cells (DLBCL) after treatment with Compound (22). ST486 cells were incubated with 10 μM, 1 μM, 100 nM, 10 nM, nM, 100 pM, 10 pM, 1 pM, and 100 fM of Compound (22) for 24 hours. Cell surface BCMA levels were observed by flow cytometry (FACS analysis).

FIG. 10G. Cell surface BCMA levels in SU-DHL cells (DLBCL) after treatment with Compound (22). SU-DHL cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

FIG. 10H. Cell surface BCMA levels in SU-DHL-10 cells (DLBCL) after treatment with Compound (22). SU-DHL-10 cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

FIG. 10I. Cell surface BCMA levels in Namalwa cells (Burkitt's Lymphoma) after treatment with Compound (22). Namalwa cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

FIG. 10J. Cell surface BCMA levels in CA46 cells (Burkitt's Lymphoma) after treatment with Compound (22). CA46 cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

FIG. 10K. Cell surface BCMA levels in EB2 cells (Burkitt's Lymphoma) after treatment with Compound (22). EB2 cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

FIG. 10L. Cell surface BCMA levels in Ramos cells (Burkitt's Lymphoma) after treatment with Compound (22). Ramos cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

FIG. 10M. Cell surface BCMA levels in GA-10 cells (Burkitt's Lymphoma) after treatment with Compound (22). GA-10 cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

FIG. 10N. Cell surface BCMA levels in Raji cells (Burkitt's Lymphoma) after treatment with Compound (22). Raji cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

FIG. 100. Cell surface BCMA levels in BC-1 cells (B-cell Lymphoma) after treatment with Compound (22). BC-1 cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

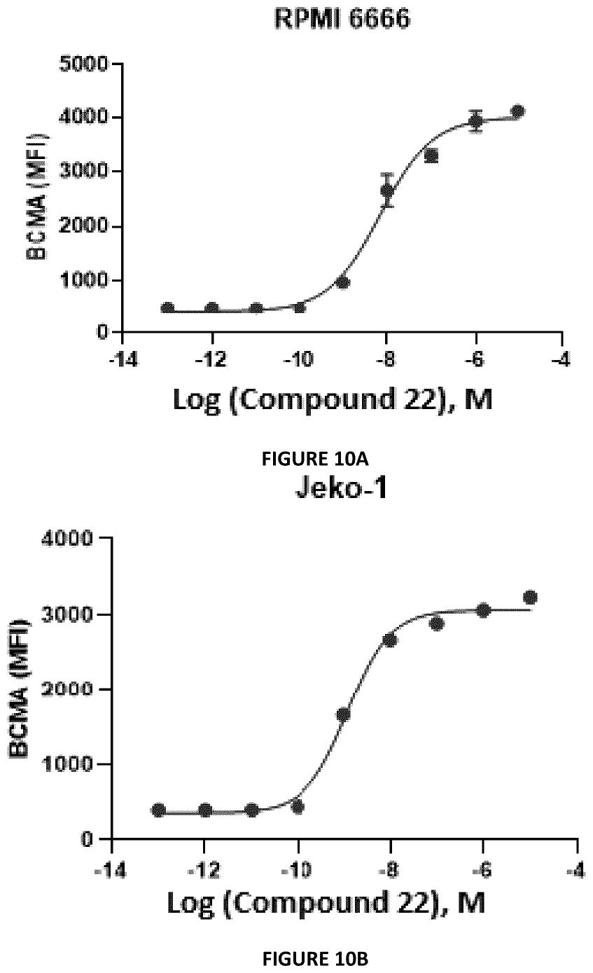

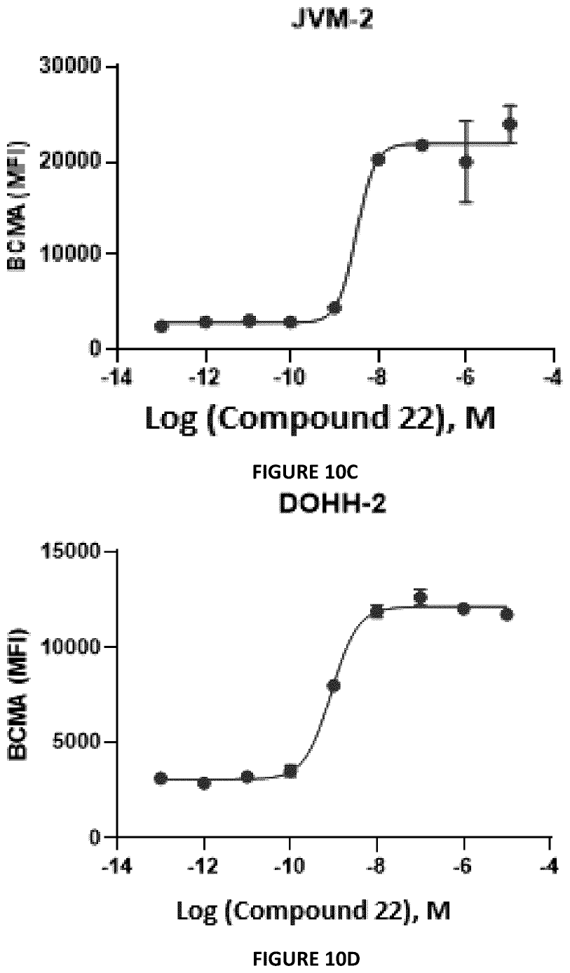

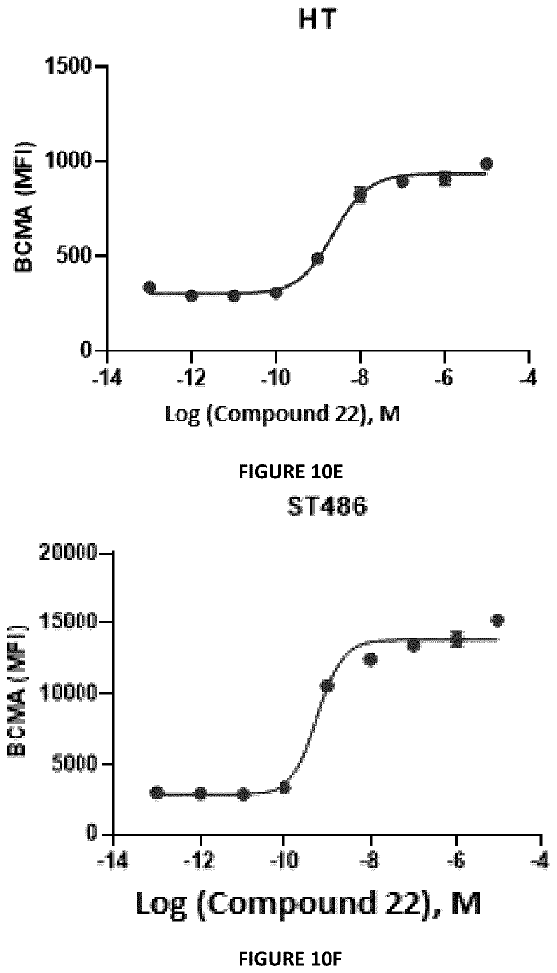

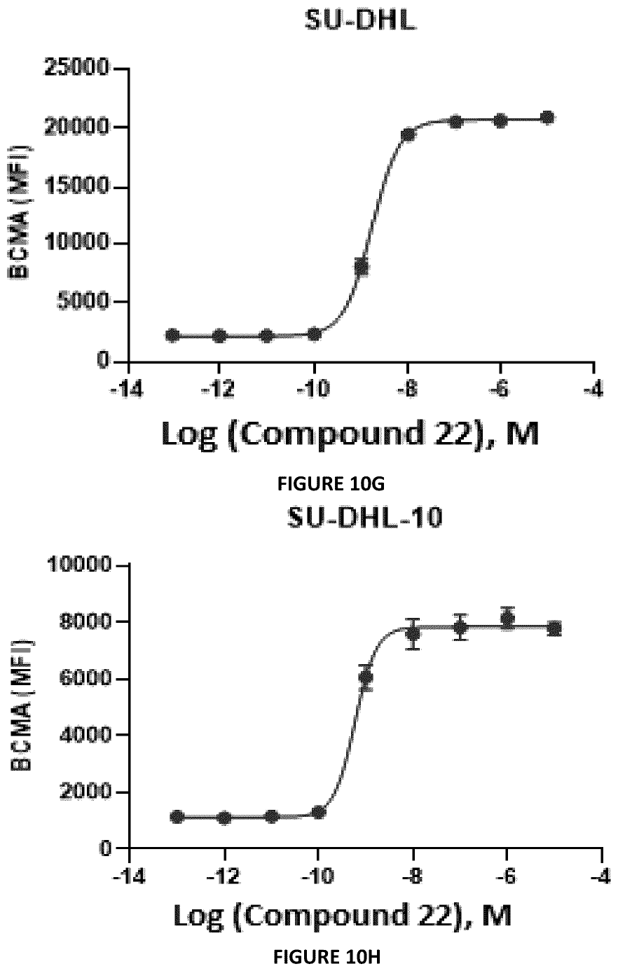

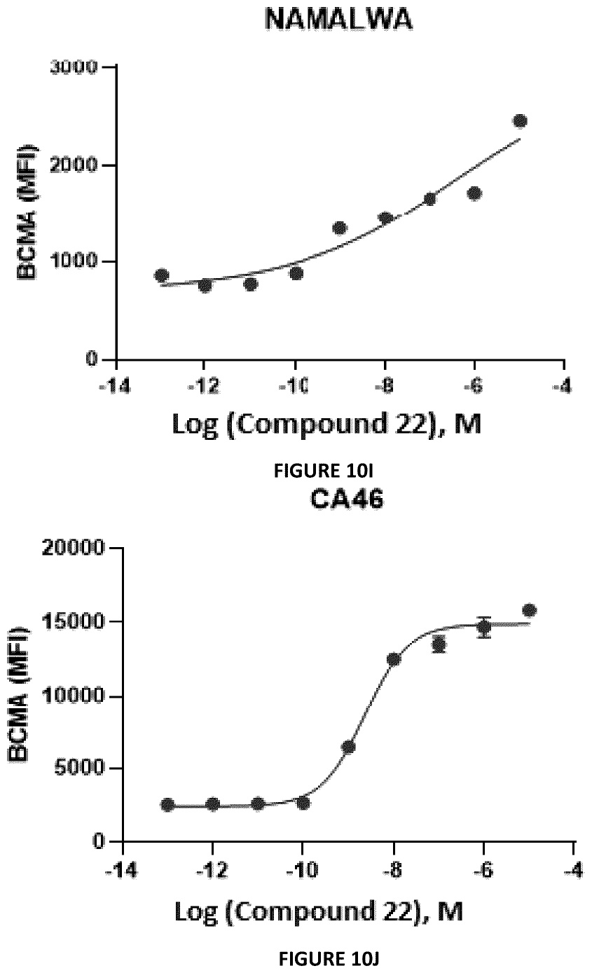

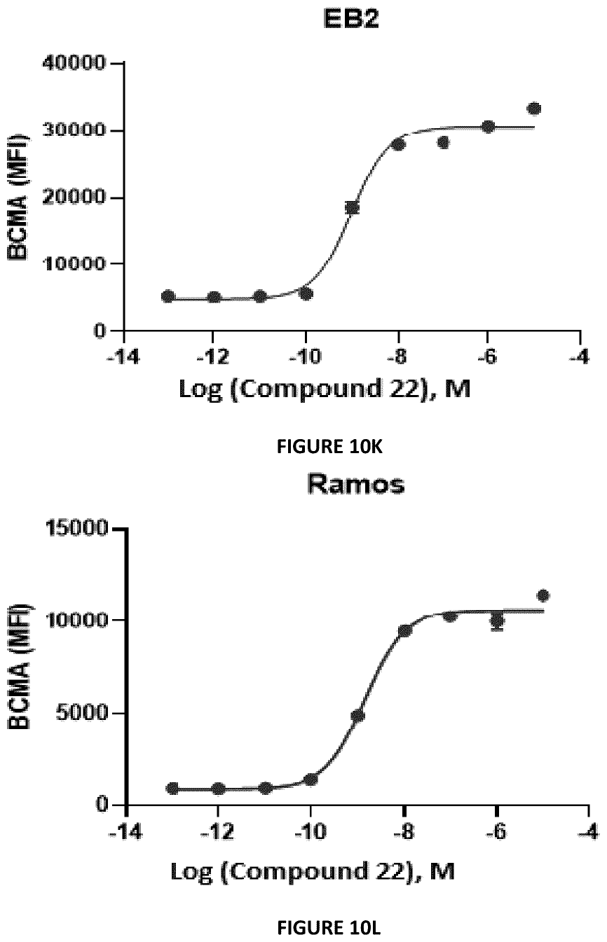

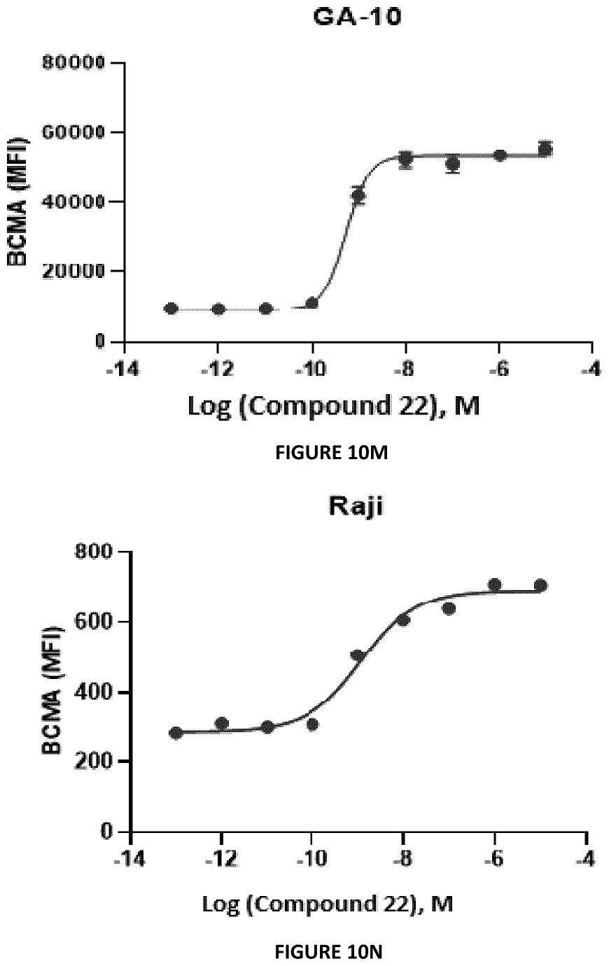

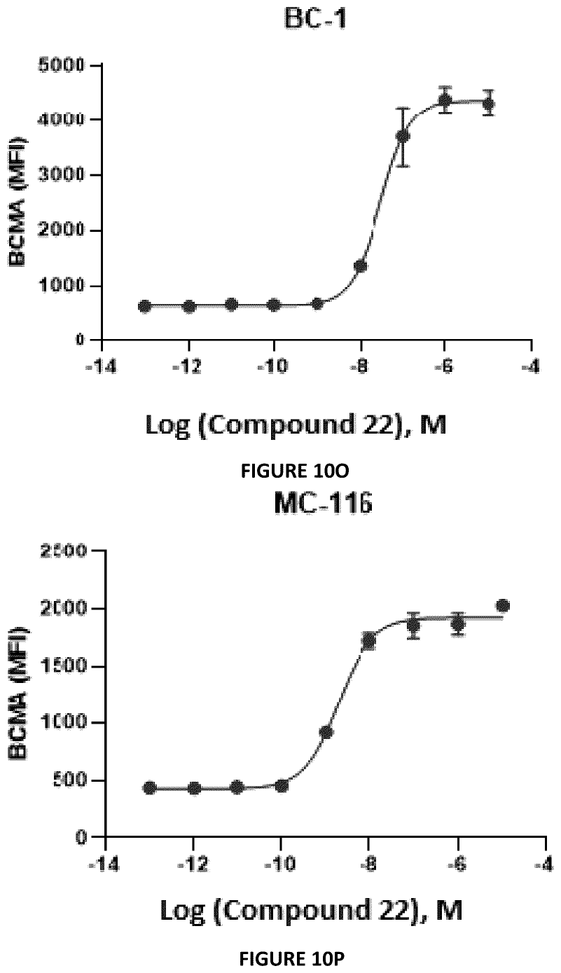

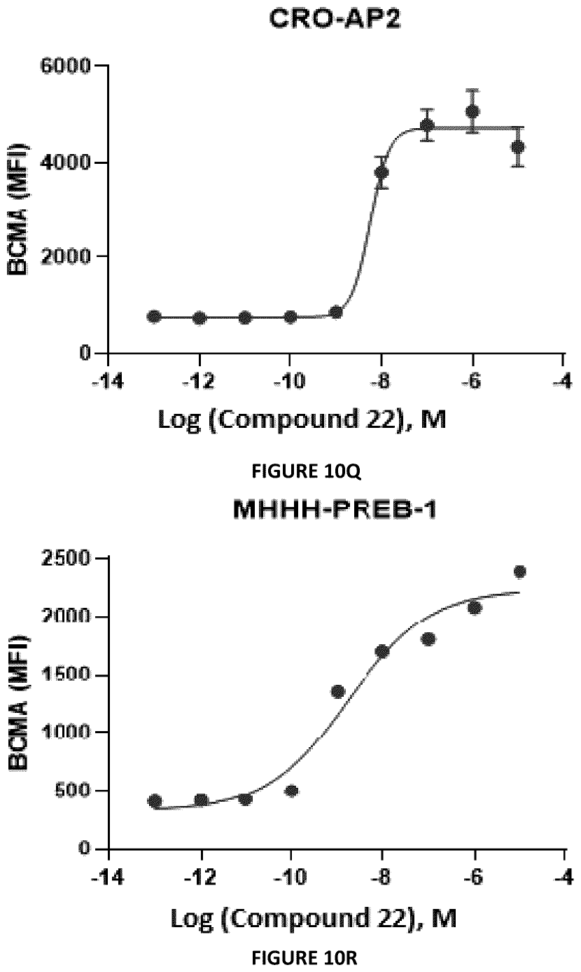

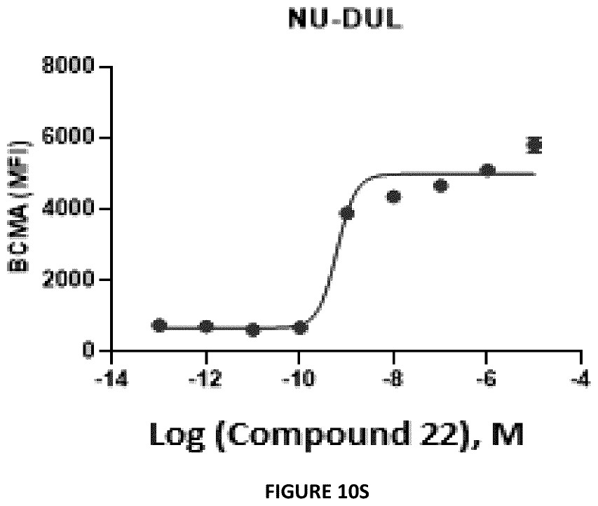

FIG. 10P. Cell surface BCMA levels in MC-116 cells (B-cell Lymphoma) after treatment with Compound (22). MC-116 cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

FIG. 10Q. Cell surface BCMA levels in CRO-AP2 cells (B-cell Lymphoma) after treatment with Compound (22). CRO-AP2 cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

FIG. 10R. Cell surface BCMA levels in MHHH-PREB-1 cells (B-cell Lymphoma) after treatment with Compound (22). MHHH-PREB-1 cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

FIG. 10S. Cell surface BCMA levels in NU-DUL cells (B-cell Lymphoma) after treatment with Compound (22). NU-DUL cells were incubated with 10 μM, 1 μM, 100 nM, 10 nM, 1 nM, 100 pM, 10 pM, 1 pM, and 100 fM of Compound (22) for 24 hours. Cell surface BCMA levels were observed by flow cytometry (FACS analysis).

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In one embodiment, compositions of the present invention or for use in the methods of the present invention comprise one or more gamma secretase inhibitors. In one embodiment, the gamma secretase inhibitor comprises a bisfluoroalkyl-1,4-benzodiazepinone compound.

Bisfluoroalkyl-1,4-benzodiazepinone Compounds

In one embodiment, the present invention provides compositions comprising compounds represented by the structure of Formula (I):

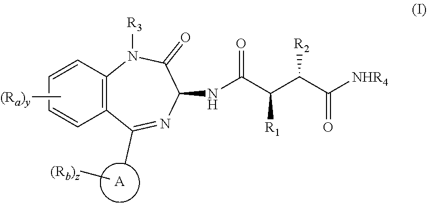

and/or at least one salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC (O)CH(NH₂)CH(CH₃)₂,

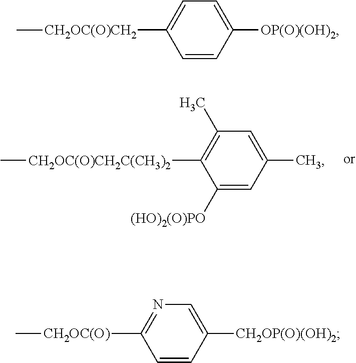

R_{y} is: —SCH₂CH(NH₂)C(O)OH, —SCH₂CH(NH₂)C(O)OCH₃, or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is zero, 1, or 2.

In one embodiment, the present invention provides compositions comprising compounds as described herein formulated at a dose of 4 mg. In one embodiment, the present invention provides compositions comprising compounds as described herein formulated for intravenous administration.

In one embodiment, the present invention provides compositions comprising compounds represented by the structure of Formula (II):

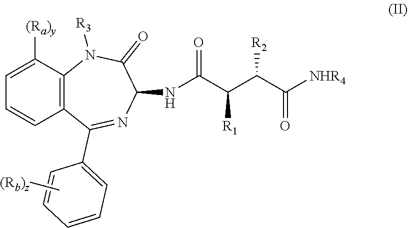

wherein R₃ is H or —CH₃; and y is zero or 1.

In one embodiment, the present invention provides compositions comprising compounds of Formula (III):

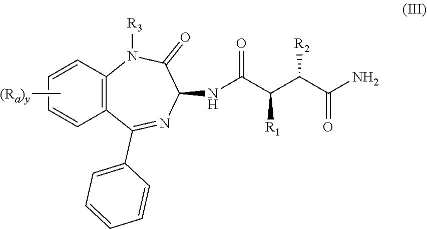

or prodrugs or salts thereof; wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H or —CH₃;
each Rₐ is independently F, Cl, —CN, —OCH₃, and/or —NHCH₂CH₂OCH₃; and
y is zero, 1, or 2.

In one embodiment, R₁ is —CH₂CF₃ or —CH₂CH₂CF₃ and R₂ is —CH₂CF₃ or —CH₂CH₂CF₃. In another embodiment, R₁ is —CH₂CH₂CF₃ and R₂ is —CH₂CH₂CF₃. In one embodiment, y is 1 or 2. In another embodiment, y is zero or 1. In another embodiment, y is zero.

In one embodiment, the compound of Formula (III) comprises: (2R,3S)—N-((3S)-1-methyl-2-oxo-5-phenyl-2,3-dihydro-1H-1,4-benzodiazepin-3-yl)-2,3-bis(3,3,3-trifluoropropyl)succinamide (1)

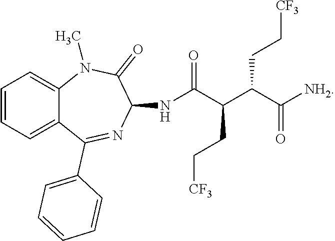 [SMILES: CN1C(=O)[C@@H](NC(=O)[C@H](CCC(F)(F)F)[C@H](CCC(F)(F)F)C(N)=O)N=C(c2ccccc2)c2ccccc21]

In another embodiment, the compound of Formula (III) comprises: (2R,3S)—N-((3S)-2-oxo-5-phenyl-2,3-dihydro-1H-1,4-benzodiazepin-3-yl)-2,3-bis(3,3,3-trifluoropropyl)succinamide (2)

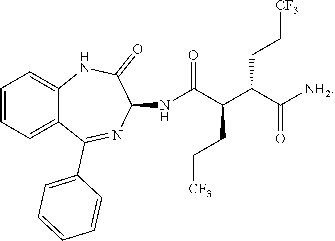 [SMILES: NC(=O)[C@@H](CCC(F)(F)F)[C@@H](CCC(F)(F)F)C(=O)N[C@H]1N=C(c2ccccc2)c2ccccc2NC1=O]

In another embodiment, the compound of Formula (III) comprises: (2R,3S)—N-((3S)-1-methyl-2-oxo-5-phenyl-2,3-dihydro-1H-1,4-benzodiazepin-3-yl)-2-(2,2,2-trifluoroethyl)-3-(3,3,3-trifluoropropyl)succinamide (3);

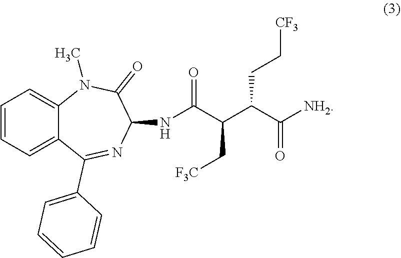 [SMILES: CN1C(=O)[C@@H](NC(=O)[C@H](CC(F)(F)F)[C@H](CCC(F)(F)F)C(N)=O)N=C(c2ccccc2)c2ccccc21]

In another embodiment, the compound of Formula (III) comprises: (2R,3S)—N-((3S)-1-methyl-2-oxo-5-phenyl-2,3-dihydro-1H-1,4-benzodiazepin-3-yl)-3-(2,2,2-trifluoroethyl)-2-(3,3,3-trifluoropropyl)succinamide (4);

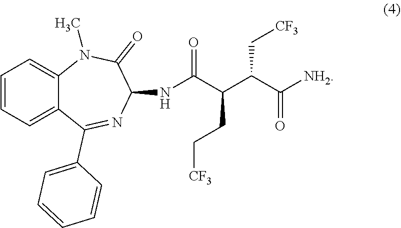 [SMILES: CN1C(=O)[C@@H](NC(=O)[C@H](CCC(F)(F)F)[C@H](CC(F)(F)F)C(N)=O)N=C(c2ccccc2)c2ccccc21]

In another embodiment, the compound of Formula (III) comprises: (2R,3S)N-((3S)-1-(²H₃)methyl-2-oxo-5-phenyl-2,3-dihydro-1H-1,4-benzodiazepin-3-yl)-2,3-bis(3,3,3-trifluoropropyl)succinamide (5);

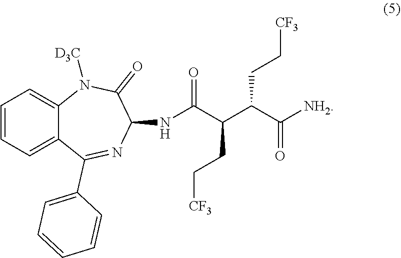 [SMILES: [2H]C([2H])([2H])N1C(=O)[C@@H](NC(=O)[C@H](CCC(F)(F)F)[C@H](CCC(F)(F)F)C(N)=O)N=C(c2ccccc2)c2ccccc21]

In another embodiment, the compound of Formula (III) comprises a compound of Formula (VI):

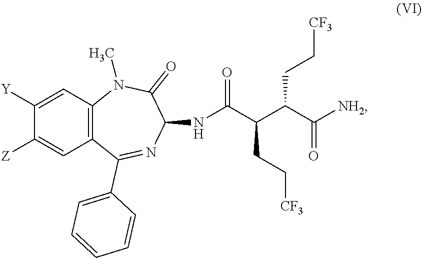

which in one embodiment, comprises (2R,3S)N-((3S)-7-chloro-1-methyl-2-oxo-5-phenyl-2,3-dihydro-1H-1,4-benzodiazepin-3-yl)-2,3-bis(3,3,3-trifluoropropyl)succinamide (6), i.e. Y═H and Z═Cl; (2R,3S)—N-((3S)-8-methoxy-1-methyl-2-oxo-5-phenyl-2,3-dihydro-1H-1,4-benzodiazepin-3-yl)-2,3-bis(3,3,3-trifluoropropyl)succinamide (7), i.e. Y═OCH₃ and Z═H; (2R,3S)—N-((3S)-8-fluoro-1-methyl-2-oxo-5-phenyl-2,3-dihydro-1H-1,4-benzodiazepin-3-yl)-2,3-bis(3,3,3-trifluoropropyl)succinamide (8), i.e. Y═F and Z═H; (2R,3S)—N-((3S)-7-methoxy-1-methyl-2-oxo-5-phenyl-2,3-dihydro-1H-1,4-benzodiazepin-3-yl)-2,3-bis(3,3,3-trifluoropropyl)succinamide (9), Y═H and Z═OCH₃; (2R,3S)—N-((3S)-7-fluoro-1-methyl-2-oxo-5-phenyl-2,3-dihydro-1H-1,4-benzodiazepin-3-yl)-2,3-bis(3,3,3-trifluoropropyl)succinamide (10), i.e. Y═H and Z═F; or (2R,3S)—N-((3S)-8-chloro-1-methyl-2-oxo-5-phenyl-2,3-dihydro-1H-1,4-benzodiazepin-3-yl)-2,3-bis(3,3,3-trifluoropropyl)succinamide (11), i.e. Y═Cl and Z═H.

In another embodiment, the compound of Formula (III) comprises a compound of Formula (VII):

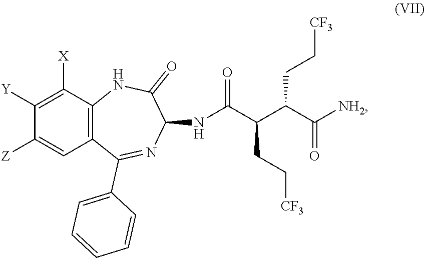

which in one embodiment, comprises (2R,3S)—N-((3S)-9-methoxy-2-oxo-5-phenyl-2,3-dihydro-1H-1,4-benzodiazepin-3-yl)-2,3-bis(3,3,3-trifluoropropyl)succinamide (12), i.e. X═OCH₃, Y═H and Z═H; (2R,3S)N-((3S)-8-methoxy-2-oxo-5-phenyl-2,3-dihydro-1H-1,4-benzodiazepin-3-yl)-2,3-bis(3,3,3-trifluoropropyl)succinamide (13), i.e. X═H, Y═OCH₃ and Z═H; (2R,3S)N-((3S)-7-methoxy-2-oxo-5-phenyl-2,3-dihydro-1H-1,4-benzodiazepin-3-yl)-2,3-bis(3,3,3-trifluoropropyl)succinamide (14), i.e. X═H, Y═H and Z═OCH₃; (2R,3S)N-((3S)-8-cyano-9-methoxy-2-oxo-5-phenyl-2,3-dihydro-1H-1,4-benzodiazepin-3-yl)-2,3-bis(3,3,3-trifluoropropyl)succinamide (15), i.e. X═OCH₃, Y═CN and Z═H; (2R,3S)N-((3S)-8,9-dichloro-2-oxo-5-phenyl-2,3-dihydro-1H-1,4-benzodiazepin-3-yl)-2,3-bis(3,3,3-trifluoropropyl)succinamide (16), i.e. X═Cl, Y═Cl and Z═H; (2R,3S)N-((3S)-9-fluoro-2-oxo-5-phenyl-2,3-dihydro-1H-1,4-benzodiazepin-3-yl)-2,3-bis(3,3,3-trifluoropropyl)succinamide (17), i.e. X═F, Y═H and Z═H; or (2R,3S)N-((3S)-9-chloro-2-oxo-5-phenyl-2,3-dihydro-1H-1,4-benzodiazepin-3-yl)-2,3-bis(3,3,3-trifluoropropyl)succinamide (18), i.e. X═Cl, Y═H and Z═H.

In another embodiment, the compound of Formula (III) comprises: (2R,3S)—N-((3S)-2-oxo-5-phenyl-2,3-dihydro-1H-1,4-benzodiazepin-3-yl)-3-(4,4,4-trifluorobutyl)-2-(3,3,3-trifluoropropyl)succinamide (19);

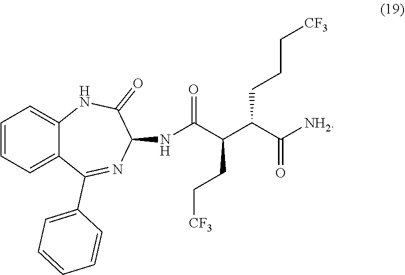 [SMILES: NC(=O)[C@@H](CCCC(F)(F)F)[C@@H](CCC(F)(F)F)C(=O)N[C@H]1N=C(c2ccccc2)c2ccccc2NC1=O]

In another embodiment, the compound of Formula (III) comprises: (2R,3S)—N-((3S)-8-methoxy-2-oxo-5-phenyl-2,3-dihydro-1H-1,4-benzodiazepin-3-yl)-3-(4,4,4-trifluorobutyl)-2-(3,3,3-trifluoropropyl)succinamide (20)

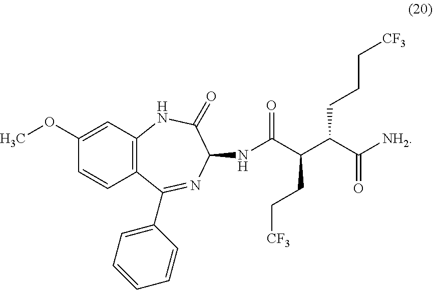 [SMILES: COc1ccc2c(c1)NC(=O)[C@@H](NC(=O)[C@H](CCC(F)(F)F)[C@H](CCCC(F)(F)F)C(N)=O)N=C2c1ccccc1]

In another embodiment, the compound of Formula (III) comprises: (2R,3S)—N-((3S)-9-((2-methoxyethyl)amino)-2-oxo-5-phenyl-2,3-dihydro-1H-1,4-benzodiazepin-3-yl)-2,3-bis(3,3,3-trifluoropropyl)succinamide (21)

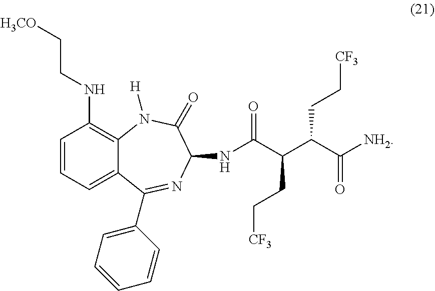 [SMILES: COCCNc1cccc2c1NC(=O)[C@@H](NC(=O)[C@H](CCC(F)(F)F)[C@H](CCC(F)(F)F)C(N)=O)N=C2c1ccccc1]

In another embodiment, the present invention provides compositions comprising compounds represented by the structure of Formula (I):

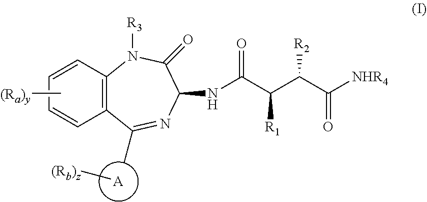

and/or at least one salt thereof, wherein:
R₁ is —CH₂CF₃;
R₂ is —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

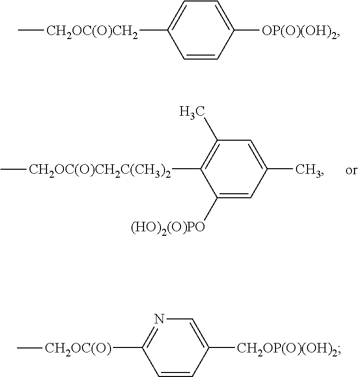

R_{y} is: —SCH₂CH(NH₂)C(O)OH, —SCH₂CH(NH₂)C(O)OCH₃, or —SCH₂CH(NH₂)C(O)OC(CH₃)₃;
Ring A is phenyl or pyridinyl;
each Rₐ is independently Cl, C₁₋₃ alkyl, —CH₂OH, —CF₃, cyclopropyl, —OCH₃, and/or —O(cyclopropyl);
each R_{b} is independently F, Cl, —CH₃, —CH₂OH, —CF₃, cyclopropyl, and/or —OCH₃;
y is zero, 1 or 2; and
z is 1 or 2.

In another embodiment, Ring A is phenyl; and R₃ is H. In another embodiment, R₂ is —CH₂CH₂CF₃; and Ring A is phenyl. In another embodiment, R₂ is —CH₂CH₂CF₃; Ring A is phenyl; Rₐ is C₁₋₃ alkyl or —CH₂OH; each Re is independently F and/or Cl; and y is 1.

In another embodiment, the present invention provides compositions comprising compounds represented by the structure of Formula (IV):

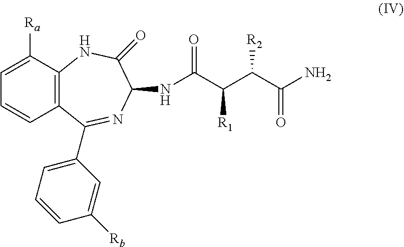

In another embodiment, the present invention provides compositions comprising compounds represented by the structure of Formula (V):

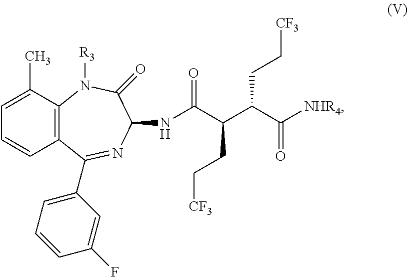

wherein R₃ is H or Rₓ.

In another embodiment, the present invention provides compositions comprising (2R,3S)—N-((3S)-5-(3-fluorophenyl)-9-methyl-2-oxo-2,3-dihydro-1H-1,4-benzodiazepin-3-yl)-2,3-bis(3,3,3-trifluoropropyl)succinamide (22); (2R,3S)—N-((3S)-5-(3-chlorophenyl)-9-ethyl-2-oxo-2,3-dihydro-1H-1,4-benzodiazepin-3-yl)-2,3-bis(3,3,3-trifluoropropyl)succinamide (23); (2R,3S)—N-((3S)-5-(3-chlorophenyl)-9-isopropyl-2-oxo-2,3-dihydro-1H-1,4-benzodiazepin-3-yl)-2,3-bis(3,3,3-trifluoropropyl)succinamide (24); (2R,3S)—N-(9-chloro-5-(3,4-dimethylphenyl)-2-oxo-2,3-dihydro-1H-1,4-benzodiazepin-3-yl)-3-(4,4,4-trifluorobutyl)-2-(3,3,3-trifluoropropyl)succinamide (25); (2R,3S)—N-(9-chloro-5-(3,5-dimethylphenyl)-2-oxo-2,3-dihydro-1H-1,4-benzodiazepin-3-yl)-3-(4,4,4-trifluorobutyl)-2-(3,3,3-trifluoropropyl)succinamide (26); (2R,3S)—N-((3S)-9-ethyl-5-(3-methylphenyl)-2-oxo-2,3-dihydro-1H-1,4-benzodiazepin-3-yl)-2,3-bis(3,3,3-trifluoropropyl)succinamide (27); (2R,3S)—N-((3S)-5-(3-chlorophenyl)-9-methyl-2-oxo-2,3-dihydro-1H-1,4-benzodiazepin-3-yl)-2,3-bis(3,3,3-trifluoropropyl)succinamide (28); (2R,3S)—N-((3S)-5-(3-chlorophenyl)-9-methyl-2-oxo-2,3-dihydro-1H-1,4-benzodiazepin-3-yl)-3-(4,4,4-trifluorobutyl)-2-(3,3,3-trifluoropropyl)succinamide (29); (2R,3S)—N-((3S)-5-(3-methylphenyl)-2-oxo-9-(trifluoromethyl)-2,3-dihydro-1H-1,4-benzodiazepin-3-yl)-2,3-bis(3,3,3-trifluoropropyl)succinamide (30); (2R,3S)—N-((3S)-9-chloro-5-(3,5-dimethylphenyl)-2-oxo-2,3-dihydro-1H-1,4-benzodiazepin-3-yl)-2,3-bis(3,3,3-trifluoropropyl)succinamide (31); (2R,3S)—N-((3S)-5-(3-methylphenyl)-2-oxo-9-(trifluoromethyl)-2,3-dihydro-1H-1,4-benzodiazepin-3-yl)-3-(4,4,4-trifluorobutyl)-2-(3,3,3-trifluoropropyl)succinamide (32); (2R,3S)—N-((3S)-9-isopropyl-5-(3-methylphenyl)-2-oxo-2,3-dihydro-1H-1,4-benzodiazepin-3-yl)-2,3-bis(3,3,3-trifluoropropyl)succinamide (33); (2R,3S)—N-((3S)-9-(cyclopropyloxy)-5-(3-methylphenyl)-2-oxo-2,3-dihydro-1H-1,4-benzodiazepin-3-yl)-3-(4,4,4-trifluorobutyl)-2-(3,3,3-trifluoropropyl)succinamide (34); (2R,3S)—N-((3S)-9-(cyclopropyloxy)-5-(3-methylphenyl)-2-oxo-2,3-dihydro-1H-1,4-benzodiazepin-3-yl)-2,3-bis(3,3,3-trifluoropropyl)succinamide (35); (2R,3S)—N-((3S)-9-chloro-5-(3-methylphenyl)-2-oxo-2,3-dihydro-1H-1,4-benzodiazepin-3-yl)-3-(4,4,4-trifluorobutyl)-2-(3,3,3-trifluoropropyl) succinamide (36); (2R,3S)—N-((3S)-9-methyl-2-oxo-5-(3-(trifluoromethyl)phenyl)-2,3-dihydro-1H-1,4-benzodiazepin-3-yl)-3-(4,4,4-trifluorobutyl)-2-(3,3,3-trifluoropropyl) succinamide (37); (2R,3S)—N-((3S)-9-methyl-2-oxo-5-(3-(trifluoromethyl) phenyl)-2,3-dihydro-1H-1,4-benzodiazepin-3-yl)-2,3-bis(3,3,3-trifluoropropyl) succinamide (38); (2R,3S)—N-((3S)-9-chloro-5-(2-methylphenyl)-2-oxo-2,3-dihydro-1H-1,4-benzodiazepin-3-yl)-2,3-bis(3,3,3-trifluoropropyl)succinamide (39); (2R,3S)—N-((3S)-5-(4-fluorophenyl)-9-methyl-2-oxo-2,3-dihydro-1H-1,4-benzodiazepin-3-yl)-2,3-bis(3,3,3-trifluoropropyl)succinamide (40); (2R,3S)—N-((3S)-9-chloro-5-(3-cyclopropylphenyl)-2-oxo-2,3-dihydro-1H-1,4-benzodiazepin-3-yl)-2,3-bis(3,3,3-trifluoropropyl)succinamide (41); (2R,3S)—N-((3S)-5-(3-chlorophenyl)-9-methoxy-2-oxo-2,3-dihydro-1H-1,4-benzodiazepin-3-yl)-2,3-bis(3,3,3-trifluoropropyl)succinamide (42); (2R,3S)—N-((3S)-5-(4-chlorophenyl)-9-methoxy-2-oxo-2,3-dihydro-1H-1,4-benzodiazepin-3-yl)-2,3-bis(3,3,3-trifluoropropyl)succinamide (43); (2R,3S)—N-((3S)-9-chloro-5-(3-methylphenyl)-2-oxo-2,3-dihydro-1H-1,4-benzodiazepin-3-yl)-2,3-bis(3,3,3-trifluoropropyl)succinamide (44); (2R,3S)—N-((3S)-5-(3-methylphenyl)-9-methoxy-2-oxo-2,3-dihydro-1H-1,4-benzodiazepin-3-yl)-2,3-bis(3,3,3-trifluoropropyl)succinamide (45); (2R,3S)—N-((3S)-5-(4-(hydroxymethyl)phenyl)-2-oxo-2,3-dihydro-1H-1,4-benzodiazepin-3-yl)-2,3-bis(3,3,3-trifluoropropyl)succinamide (46); (2R,3S)—N-((3S)-5-(2-methylphenyl)-2-oxo-2,3-dihydro-1H-1,4-benzodiazepin-3-yl)-2,3-bis(3,3,3-trifluoropropyl)succinamide (47); (2R,3S)—N-((3S)-5-(3-methylphenyl)-2-oxo-2,3-dihydro-1H-1,4-benzodiazepin-3-yl)-2,3-bis(3,3,3-trifluoropropyl)succinamide (48); (2R,3S)—N-((3S)-9-methoxy-2-oxo-5-(5-(trifluoromethyl)-2-pyridinyl)-2,3-dihydro-1H-1,4-benzodiazepin-3-yl)-2,3-bis(3,3,3-trifluoropropyl)succinamide (49); (2R,3S)—N-((3S)-5-(5-chloro-2-pyridinyl)-9-methoxy-2-oxo-2,3-dihydro-1H-1,4-benzodiazepin-3-yl)-2,3-bis(3,3,3-trifluoropropyl)succinamide (50); (2R,3S)—N-((3S)-5-(4-methoxyphenyl)-2-oxo-2,3-dihydro-1H-1,4-benzodiazepin-3-yl)-2,3-bis(3,3,3-trifluoropropyl)succinamide (51); (2R,3S)—N-((3S)-5-(4-methylphenyl)-2-oxo-2,3-dihydro-1H-1,4-benzodiazepin-3-yl)-2,3-bis(3,3,3-trifluoropropyl)succinamide (52); (2R,3S)—N-((3S)-5-(3-fluorophenyl)-9-(hydroxymethyl)-2-oxo-2,3-dihydro-1H-1,4-benzodiazepin-3-yl)-2,3-bis(3,3,3-trifluoropropyl)succinamide (53); ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl L-valinate (54); ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl L-alaninate (55); S-(((2S,3R)-6,6,6-trifluoro-3-(((3S)-5-(3-fluorophenyl)-9-methyl-2-oxo-2,3-dihydro-1H-1,4-benzodiazepin-3-yl)carbamoyl)-2-(3,3,3-trifluoropropyl)hexanoyl)amino)-L-cysteine (56); tert-butyl S-(((2S,3R)-6,6,6-trifluoro-3-(((3S)-5-(3-fluorophenyl)-9-methyl-2-oxo-2,3-dihydro-1H-1,4-benzodiazepin-3-yl)carbamoyl)-2-(3,3,3-trifluoropropyl)hexanoyl)amino)-L-cysteinate (57); methyl S-(((2S,3R)-6,6,6-trifluoro-3-(((3S)-5-(3-fluorophenyl)-9-methyl-2-oxo-2,3-dihydro-1H-1,4-benzodiazepin-3-yl)carbamoyl)-2-(3,3,3-trifluoropropyl) hexanoyl)amino)-L-cysteinate (58); ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl (4-(phosphonooxy)phenyl)acetate (59); and ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl L-valyl-L-valinate (60); and salts thereof.

In another embodiment, the present invention provides compositions comprising compounds represented by the structure of Formula (I):

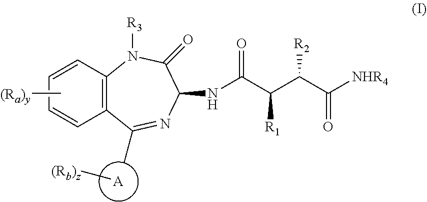

and/or at least one salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC (O)CH(NH₂)CH(CH₃)₂,

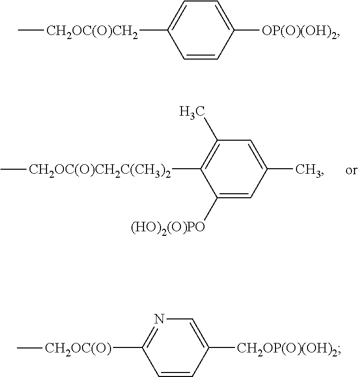

R_{y} is: —SCH₂CH(NH₂)C(O)OH, —SCH₂CH(NH₂)C(O)OCH₃, or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is zero, 1, or 2
provided that if Ring A is phenyl, z is zero, and y is 1 or 2 then at least one Rₐ is C₁₋₃ alkyl, —CH₂OH, —CF₃, cyclopropyl, or —O(cyclopropyl);
provided that if R₃ is Rₓ then R₄ is H; and
provided that if R₄ is R_{y} then R₃ is H or —CH₃.

In another embodiment, the structure as described hereinabove comprises one or more of the following provisos: provided that if Ring A is phenyl, z is zero, and y is 1 or 2 then at least one Rₐ is C₁₋₃ alkyl, —CH₂OH, —CF₃, cyclopropyl, or —O(cyclopropyl); provided that if R₃ is Rₓ then R₄ is H; and provided that if R₄ is R_{y} then R₃ is H or —CH₃.

In another embodiment, the present invention provides compositions comprising compounds represented by the following structure:

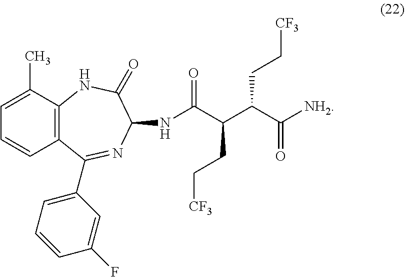 [SMILES: Cc1cccc2c1NC(=O)[C@@H](NC(=O)[C@H](CCC(F)(F)F)[C@H](CCC(F)(F)F)C(N)=O)N=C2c1cccc(F)c1]

In another embodiment, the compounds as described herein comprise prodrugs of one or more of the compounds.

U.S. Pat. No. 9,273,014, which is incorporated herein in its entirety, discloses various compounds of Formula (I):

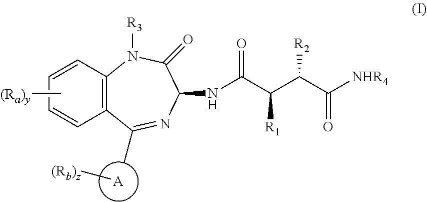

and/or at least one salt thereof, wherein:
R₁ is —CH₂CH₂CF₃;
R₂ is —CH₂CH₂CF₃ or —CH₂CH₂CH₂CF₃;
R₃ is H, —CH₃, or Rₓ;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC (O)CH(NH₂)CH(CH₃)₂,

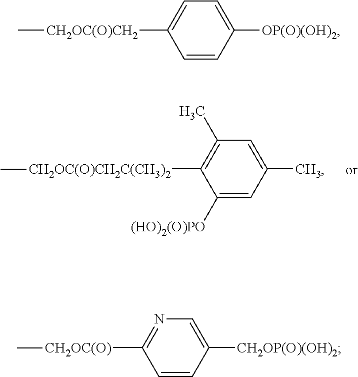

R_{y} is: —SCH₂CH(NH₂)C(O)OH, —SCH₂CH(NH₂)C(O)OCH₃, or —SCH₂CH(NH₂)C(O)OC(CH₃)₃;
Ring A is phenyl or pyridinyl;
each Rₐ is independently Cl, C₁₋₃ alkyl, —CH₂OH, —CF₃, cyclopropyl, —OCH₃, and/or —O(cyclopropyl);
each R_{b} is independently F, Cl, —CH₃, —CH₂OH, —CF₃, cyclopropyl, and/or —OCH₃;
y is zero, 1, or 2; and
z is 1 or 2.

U.S. Pat. No. 9,273,014 also discloses the compound of Formula (22):

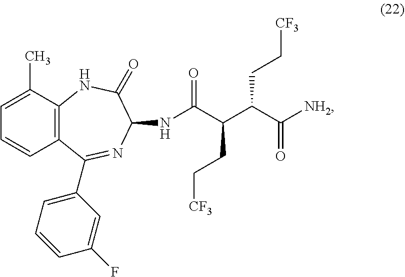 [SMILES: Cc1cccc2c1NC(=O)[C@@H](NC(=O)[C@H](CCC(F)(F)F)[C@H](CCC(F)(F)F)C(N)=O)N=C2c1cccc(F)c1]

which, in one embodiment, has the chemical name (2R,3S)—N-((3S)-5-(3-fluorophenyl)-9-methyl-2-oxo-2,3-dihydro-1H-1,4-benzodiazepin-3-yl)-2,3-bis(3,3,3-trifluoropropyl)succinamide. U.S. Pat. No. 9,273,014 also discloses a process for synthesizing the compounds as well as other compounds of Formula (I), which are to be considered as part of the present invention.

U.S. Pat. No. 8,629,136, which is incorporated by reference herein in its entirety, discloses compounds of Formula (III):

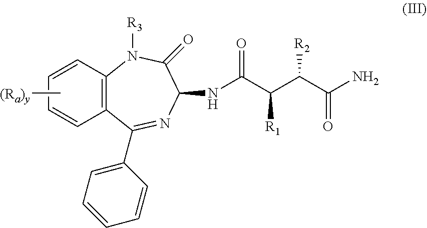

and/or at least one salt thereof, wherein:
R₃ is H or —CH₃; and
each Rₐ is independently F, Cl, —CN, —OCH₃ and/or —NHCH₂CH₂OCH₃.

U.S. Pat. No. 8,629,136 also discloses the compound of Formula (1):

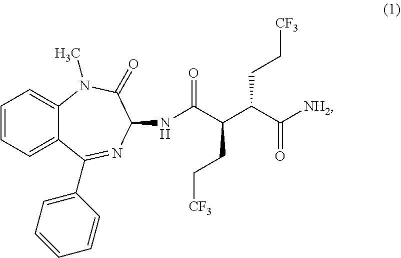 [SMILES: CN1C(=O)[C@@H](NC(=O)[C@H](CCC(F)(F)F)[C@H](CCC(F)(F)F)C(N)=O)N=C(c2ccccc2)c2ccccc21]

which, in one embodiment, has the chemical name (2R,3S)—N-((3S)-1-methyl-2-oxo-5-phenyl-2,3-dihydro-1H-1,4-benzodiazepin-3-yl)-2,3-bis(3,3,3-trifluoropropyl)succinamide. In one embodiment, the compounds are Notch inhibitors. U.S. Pat. No. 8,629,136 discloses a process for synthesizing the compounds as well as other compounds of Formula (I), which are to be considered as part of the present invention.

The present invention may be embodied in other specific forms without departing from the spirit or essential attributes thereof. This invention encompasses all combinations of the aspects and/or embodiments of the invention noted herein. It is understood that any and all embodiments of the present invention may be taken in conjunction with any other embodiment or embodiments to describe additional embodiments. It is also to be understood that each individual element of the embodiments is meant to be combined with any and all other elements from any embodiment to describe an additional embodiment.

In one embodiment, compositions of the present invention or for use in the methods of the present invention comprise one or more chimeric antigen receptor T cells (CAR-T cells).

### Chimeric Antigen Receptor T Cells (CAR-T Cells)

Chimeric antigen receptors (CARs) are proteins that incorporate an antigen recognition domain, costimulatory domains, and T-cell activation domains. T cells genetically modified to express CARs specifically recognize and eliminate malignant cells expressing a targeted antigen.

CAR-T cell therapy is the second U.S. Food and Drug Administration approved gene therapy. However, the efficacy of CAR-T cells in treating tumors has been unpredictable, because tumors develop mechanisms to evade elimination by the immune system such as via suppression of local immune cell activity. Therefore, CAR-T cells as a stand-alone treatment may be limited. However, combining CAR-T cell therapy with other compounds, such as the combinations described herein, may significantly increase the efficacy of CAR-T cells therapy.

In one embodiment, chimeric antigen receptors (CARs) are a type of antigen-targeted receptor composed of intracellular T-cell signaling domains fused to extracellular tumor-binding moieties, most commonly single-chain variable fragments (scFvs) from monoclonal antibodies. CARs directly recognize cell surface antigens, independent of MHC-mediated presentation, permitting the use of a single receptor construct specific for any given antigen in all patients. Initial CARs fused antigen-recognition domains to the CD3(activation chain of the T-cell receptor (TCR) complex. While these first generation CARs induced T-cell effector function in vitro, they were largely limited by poor antitumor efficacy in vivo. Subsequent CAR iterations have included secondary costimulatory signals in tandem with CD3ζ, including intracellular domains from CD28 or a variety of TNF receptor family molecules such as 4-1BB (CD137) and OX40 (CD134). Further, third generation receptors include two costimulatory signals in addition to CD3ζ, most commonly from CD28 and 4-1BB. Second and third generation CARs dramatically improved antitumor efficacy, in some cases inducing complete remissions in a proportion of patients with advanced cancer. In one embodiment, the 4-1BB domain is optimized for less acute toxicity and more durable CAR T cell persistence.

In one embodiment, a CAR-T cell is an immunoresponsive cell comprising an antigen receptor, which is activated when its receptor binds to its antigen.

In one embodiment, the CAR-T cells used in the compositions and methods as disclosed herein are first generation CAR-T cells. In another embodiment, the CAR-T cells used in the compositions and methods as disclosed herein are second generation CAR-T cells. In another embodiment, the CAR-T cells used in the compositions and methods as disclosed herein are third generation CAR-T cells. In another embodiment, the CAR-T cells used in the compositions and methods as disclosed herein are fourth generation CAR-T cells.

In one embodiment, CAR-modified T-cell potency may be further enhanced through the introduction of additional genes, including those encoding proliferative cytokines (i.e., IL-12) or costimulatory ligands (ie, 4-1BBL), thus producing “armored” fourth-generation CAR-modified T-cells. In one embodiment, “armored CAR-T cells,” are CAR-T cells which are protected from the inhibitory tumor microenvironment. In another embodiment, the “armored” CAR technology incorporates the local secretion of soluble signaling proteins to amplify the immune response within the tumor microenvironment with the goal of minimizing systemic side effects. In one embodiment, the signaling protein signal is IL-12, which can stimulate T-cell activation and recruitment. In one embodiment, “armored” CAR technology is especially useful in solid tumor indications, in which microenvironment and potent immunosuppressive mechanisms have the potential to make the establishment of a robust anti-tumor response more challenging.

In one embodiment, CAR T-cells are genetically modified to encode molecules involved in the prevention of apoptosis, the remodeling of the tumor microenvironment, induction of homeostatic proliferation, and chemokine receptors that promote directed T-cell homing.

In another embodiment, CAR T-cell therapy used in the compositions and methods as disclosed herein is enhanced using the expression of cytokine transgenes, combination therapy with small molecule inhibitors, or monoclonal antibodies. In another embodiment, other strategies aimed at improving CAR T-cell therapy including using dual CARs and chemokine receptors to more specifically target tumor cells are to be considered part of the CAR T-cells and CAR T-cell therapy as disclosed herein.

In one embodiment, the CAR T-cells of the compositions and methods as disclosed herein comprise a second binding domain that can lead to either an inhibitory or amplifying signal, in order to increase specificity of CAR T-cells for cancer cells versus normal cells. For example, a CAR T-cell can be engineered such that it would be triggered in the presence of one target protein, but if a second protein is present it would be inhibited. Alternatively, it could also be engineered such that two target proteins would be required for maximal activation. These approaches may increase the specificity of the CAR for tumor relative to normal tissue.

In another embodiment, T cells redirected for universal cytokine killing (TRUCK) may be used. In one embodiment, TRUCK is a way to redirect CAR-T cells by producing and releasing a transgenic product, such as IL-12, to activate innate immune response against tumor cells which are invisible to CAR-T cells. In another embodiment, CAR OT-I cells which induce serial killing in addition to recognizing target tumor cells and secreting cytotoxic granule proteins (perforin, granzyme B). In another embodiment, CAR-T cells are engineered to produce interferon gamma. In another embodiment, CAR-T cell function is improved through co-activation of macrophage and NK cells.

In one embodiment, the CAR T-cells used in the compositions and methods as disclosed herein encode antibody-based external receptor structures and cytosolic domains that encode signal transduction modules composed of the immunoreceptor tyrosine-based activation motif.

Accordingly, one embodiment as disclosed herein relates to cytotoxic immune cells (e.g., NK cells or T-cells) comprising chimeric antigen receptors (CARs) whereby the cells retain their cytotoxic function. In another embodiment, the chimeric antigen receptor is exogenous to the T-cell. In another embodiment, the CAR is recombinantly expressed. In another embodiment, the CAR is expressed from a vector.

In one embodiment, the T-cell utilized to generate CAR T-cells is a naïve CD4⁺ T-cell. In another embodiment, the T-cell utilized to generate CAR T-cells is a naïve CD8⁺ T-cell. In another embodiment, the T-cell utilized to generate CAR T-cells is an effector T-cell. In another embodiment, the T-cell utilized to generate CAR T-cells is a regulatory T-cell (Treg). In another embodiment, the T-cell utilized to generate CAR T-cells is a cytotoxic T-cell. In another embodiment, the CAR-T cells comprise a high proportion of stem cell memory T cells (T_{SCM}).

In one embodiment, disclosed herein are compositions comprising genetically modified immune cells. In another embodiment, the genetically modified immune cell is a T-cell. In another embodiment, a T-cell is a naïve T-cell. In another embodiment, a T-cell is a naïve CD4⁺ T-cell. In another embodiment, a T-cell is a naïve CD8⁺ T-cell. In another embodiment, the genetically modified immune cell is a natural killer (NK) cell. In another embodiment, the genetically modified immune cell is a dendritic cell. In still another embodiment, the genetically modified T-cell is a cytotoxic T lymphocyte (CTL cell). In another embodiment, the genetically modified T-cell is a regulatory T-cell (Treg). In another embodiment, the genetically modified T-cell is a chimeric antigen receptor (CAR) T-cell. In another embodiment, the genetically modified T-cell is a genetically modified T-cell receptor (TCR) cell.

In one embodiment, the immune cells are cytotoxic. In another embodiment, cytotoxic cells for genetic modification can be obtained from bone marrow of the subject (autologous) or a donor (allogeneic). In other cases, the cells are obtained from a stem cell. For example, cytotoxic cells can be derived from human pluripotent stem cells such as human embryonic stem cells or human induced pluripotent T-cells. In the case of induced pluripotent stem cells (IPSCs), such pluripotent T-cells can be obtained using a somatic cell from the subject to which genetically modified cytotoxic cells will be provided. In one embodiment, immune cells may be obtained from a subject or donor by harvesting cells by venipuncture, by apheresis methods, by white cell mobilization followed by apheresis or venipuncture, or by bone marrow aspiration.

In one embodiment, immune cells, for example T-cell, are generated and expanded by the presence of specific factors in vivo. In another embodiment, T-cell generation and maintenance is affected by cytokines in vivo. In another embodiment, cytokines that affect generation and maintenance to T-helper cells in vivo comprise IL-1, IL-2, IL-4, IL-6, IL-12, IL-21, IL-23, IL-25, IL-33, and TGFβ.

CAR T-cells have been described extensively in the literature, see for example Themelli et al. (2015) New Cell Sources for T Cell Engineering and Adoptive Immunotherapy. Cell Stem Cell 16: 357-366; Sharpe and Mount (2015) Genetically modified T cells in cancer therapy: opportunities and challenges. Disease Models & Mechanisms 8:337-350; Han et al. (2013) Journal of Hematology & Oncology 6:47-53; Wilkie et al. (2010) J Bio Chem 285(33):25538-25544; and van der Stegen et al. (2013) J. Immunol 191: 4589-4598. CAR T-cells are available to order from a commercial source such as Creative Biolabs (NY USA), which provides custom construction and production services for Chimeric Antigen Receptors (CAR) and also provides premade CAR constructs stock, which can induce protective immunity encode by recombinant adenovirus vaccine.

### T-Cell Receptor (TCR) Cells

In one embodiment, compositions and methods as disclosed herein utilize a designer T-cell receptor (TCR) cells in addition to or in place of CAR T-cells. The TCR is a multi-subunit transmembrane complex that mediates the antigen-specific activation of T-cells. The TCR is composed of two different polypeptide chains. The TCR confers antigenic specificity on the T cell, by recognizing an antigen epitope on the target cell, for example a tumor or cancer cell. Following contact with the antigen present on the tumor or cancer cell, T-cells proliferate and acquire the phenotype and function to allow them to eliminate the cancer or tumor cells.

In one embodiment, TCR T-cell therapy comprises introducing a T-cell receptor (TCR) that is specific to an epitope of a protein of interest into a T-cell. In another embodiment, the protein of interest is a tumor-associated antigen. In another embodiment, the genetically engineered TCR recognizes a tumor antigen epitope presented by the major histocompatibility complex (MHC) on the tumor cell along with T-cell activating domains. In another embodiment, the T-cell receptors recognize antigens irrespective of their intracellular or membrane localization. In another embodiment, TCRs recognize tumor cells that intracellularly express a tumor associated antigen. In one embodiment TCRs recognize internal antigens. In another embodiment, TCRs recognize angiogenic factors. In another embodiment, an angiogenic factor is a molecule involved in the formation of new blood vessels. Various genetically modified T-cell receptors and methods of their production are known in the art.

In one embodiment, TCR T-cell therapy is used to treat, prevent, inhibit, ameliorate, reduce the incidence of, or alleviate a cancer or a tumor. In one embodiment, TCR T-cell therapy is used to treat, prevent, inhibit, ameliorate, reduce the incidence of, or alleviate advanced metastatic disease, including those with hematological (lymphoma and leukemia) and solid tumors (refractory melanoma, sarcoma). In one embodiment, the TCR T-cell therapy used in the compositions and methods as disclosed herein treat a malignancy listed in Table 1 of Sadelain et al., (Cancer Discov. 2013 April; 3(4): 388-398), which is incorporated herein by reference in its entirety.

In another embodiment, the T-cell receptor is genetically modified to bind NY-ESO-1 epitopes, and the TCR-engineered T-cell is anti-NY-ESO-1. In another embodiment, the T-cell receptor is genetically modified to bind HPV-16 E6 epitopes, and the TCR-engineered T-cell is anti-HPV-16 E6. In another embodiment, the T-cell receptor is genetically modified to bind HPV-16 E7 epitopes, and the TCR-engineered T-cell is anti-HPV-16 E7. In another embodiment, the T-cell receptor is genetically modified to bind MAGE A3/A6 epitopes, and the TCR-engineered T-cell is anti-MAGE A3/A6. In another embodiment, the T-cell receptor is genetically modified to bind MAGE A3 epitopes, and the TCR-engineered T-cell is anti-MAGE A3. In another embodiment, the T-cell receptor is genetically modified to bind SSX2 epitopes, and the TCR-engineered T-cell is anti-SSX2. In another embodiment, the T-cell receptor is genetically modified to bind a target antigen disclosed herein. Using the tools well known in the art, a skilled would appreciate that the T-cell receptor may be genetically modified to bind a target antigen present on a cancer or tumor cell, wherein the TCR-engineer T-cell comprises an anti-tumor or anti-cancer cell.

In one embodiment, a method as disclosed herein comprises obtaining immune cells from a subject, and genetically modifying the immune cells to express a recombinant T-cell receptor (TCR). In another embodiment, a method as disclosed herein comprises obtaining immune cells from a subject, genetically modifying the immune cells to express a recombinant TCR and combining with an additional agent, wherein said additional agent comprises an apoptotic cell population, an apoptotic cell supernatant, a CTLA-4 blocking agent, an alpha-1 anti-trypsin or fragment thereof or analogue thereof, a tellurium-based compound, or an immune modulating agent, or any combination thereof.

TCR T-cells have been described extensively in the literature, see for example Sharpe and Mount (2015) ibid.; Essand M, Loskog ASI (2013) Genetically engineered T cells for the treatment of cancer (Review). J Intern Med 273: 166-181; and Kershaw et al. (2014) Clinical application of genetically modified T cells in cancer therapy. Clinical & Translational Immunology 3:1-7.

### Other Immunotherapeutics

In another embodiment, the present invention provides a composition comprising one or more compounds represented by the structure of Formula (I) as described herein in combination with a cancer vaccine. In another embodiment, the present invention provides a composition comprising one or more compounds represented by the structure of Formula (I) as described herein and an immunotherapeutic. In one embodiment, the immunotherapeutic comprises a monoclonal antibody.

In another embodiment, the present invention provides a composition comprising one or more B-cell maturation antigen (BCMA)-targeting immuno-therapeutics, one or more immunotherapeutic compounds, and one or more compounds represented by the structure of Formula (I):

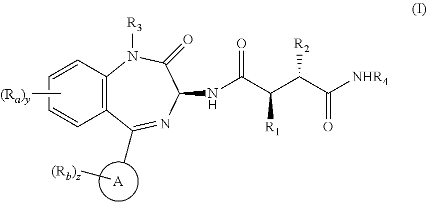

and/or at least one salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

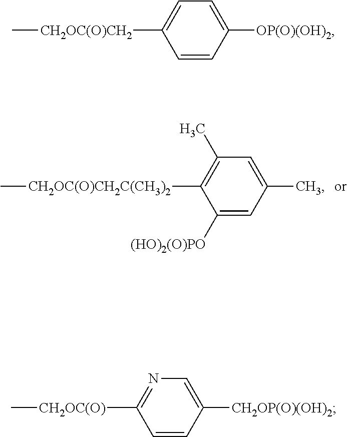

R_{y} is: —SCH₂CH(NH₂)C(O)OH, —SCH₂CH(NH₂)C(O)OCH₃, or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is zero, 1, or 2.

In another embodiment, z is 1 or 2.

In one embodiment, the BCMA-targeting immunotherapeutic comprises an antibody drug conjugate, bispecific antibody, tri-specific antibody, trifunctional antibody, chemically linked Fab or a bi-specific T-cell engager (BiTE).

In another embodiment, the present invention also provides a method as described herein comprising the step of administering to a subject a first composition comprising one or more B-cell maturation antigen (BCMA)-targeting immuno-therapeutics, a second composition comprising one or more immunotherapeutic compounds, and a third composition comprising one or more compounds represented by the structure of Formula (I):

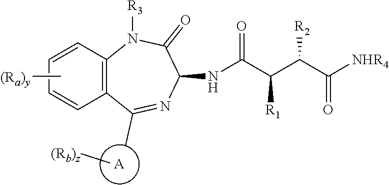

and/or at least one salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

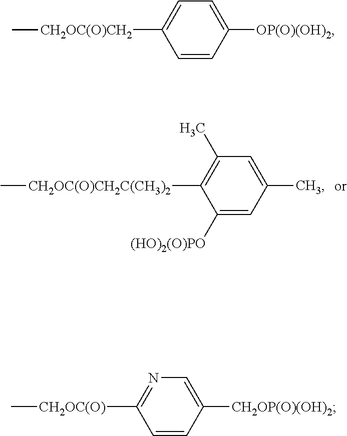

R_{y} is: —SCH₂CH(NH₂)C(O)OH, —SCH₂CH(NH₂)C(O)OCH₃, or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is zero, 1, or 2.

In another embodiment, z is 1 or 2.

### Targeting Antigens

In one embodiment, the CAR or TCR binds to an epitope of an antigen via an antibody or an antibody fragment that is directed to the antigen. In another embodiment, the antibody is a monoclonal antibody. In another embodiment, the antibody is a polyclonal antibody. In another embodiment, the antibody fragment is a single-chain variable fragment (scFv).

Appropriate target antigens for CAR T-cell therapies should be uniformly expressed on the malignancy to be treated and should not be expressed on normal essential cells. B-cell maturation antigen (BCMA) is such an appropriate target antigen. BCMA is a member of the tumor necrosis factor superfamily. Among hematologic cells, BCMA is only expressed by some B cells, normal plasma cells, and malignant plasma cells; BCMA is not expressed by hematopoietic stem cells. BCMA is uniformly expressed by the malignant plasma cells of many cases of multiple myeloma (MM) and that BCMA is not expressed by normal essential nonhematopoietic tissues. Thus, in one embodiment, the CAR-T target antigen is BCMA.

BCMA targeting methods that may be used in combination with one or more compounds represented by the structure of Formula (I) as described herein are known in the art and include BCMA-directed CAR-T, antibody drug conjugate, bispecific antibody, and tri-specific antibody.

BCMA CAR-T cells are known in the art and are described in, for example, WO2016090320A1, which is incorporated herein by reference in its entirety. In another embodiment, BCMA CAR-T cells from Novartis in collaboration with University of Pennsylvania (in one embodiment, CAR-T BCMA cells, in another embodiment, MTV273, in another embodiment, MCM998), Unicar-Therapy Biomedicine (in one embodiment, CD19 and BCMA-specific CAR-T cells), Bluebird & Celgene (in one embodiment, Bb2121, in another embodiment, Bb21217, in another embodiment, JCARH123), Gilead/Kite (in one embodiment, KITE-585), Poseida Therapeutics Inc. (in one embodiment, P-BCMA-101), CARsgen therapeutics Co. Ltd (in one embodiment, CAR-BCMA), Celgene (Juno) (in one embodiment, JCARH125), Cellectis, Allogene & Pfizer Alliance, National Institutes of Health (in one embodiment, CAR-BCMA), Autolus Ltd. (in one embodiment, APRIL CAR (AUTO02)), Celyad, or a combination thereof may be used in the present invention.

In one embodiment, the CAR-T cell has more than one target. In one embodiment, the CAR-T cell has two targets. In one embodiment, the two targets comprise BCMA and Transmembrane activator and CAML interactor (TACI).

In one embodiment, an antibody-drug conjugate for the compositions and methods as described herein comprise a chemotherapy drug. In one embodiment, the antibody-drug conjugate is from Bluebird & Celgene (Sutro) (in one embodiment, BCMA ADC).

In one embodiment, the drug in the antibody-drug conjugate comprises anti mitotic agent Monomethyl auristatin F (MMAF). In one embodiment, the antibody-drug conjugate comprising MMAF comprises GSK2857916 from GSK.

In one embodiment, the drug in the antibody-drug conjugate comprises Amanitin, which in one embodiment, kills dividing and quiescent tumor cells; specific and effective inhibitor of eukaryotic transcription, specifically inhibits RNA polymerase II, and has low toxicity of free toxin due to low membrane permeability. In one embodiment, the antibody-drug conjugate comprising Amanitin is HDP-101 from Heidelberg Pharma AG.

In one embodiment, the antibody-drug conjugate is linked via a maleimidocaproyl linker. In one embodiment the antibody-drug conjugate comprising a maleimidocaproyl linker comprises GSK2857916 from GSK.

In one embodiment, a bispecific antibody targeting BCMA for the compositions and methods of the present invention comprises a bispecific antibody from Novarts (in one embodiment, WVT078). In another embodiment, a bispecific antibody targeting BCMA for the compositions and methods of the present invention comprises a bispecific antibody from Bluebird & Celgene (in one embodiment, CC93569). In one embodiment, the bispecific antibody comprises a T cell bispecific antibody. In another embodiment, the bispecific antibody comprises a trifunctional antibody, chemically linked Fab or a bi-specific T-cell engager (BiTE). In one embodiment, the T cell engager targets CD3 T cells and BCMA. In one embodiment, the T cell engager comprises CC-93269. In another embodiment, the T cell engager comprises AMG 420 (BI836909) of Amgen (BI). In another embodiment, the T cell engager comprises AMG 701 of Amgen. In another embodiment, the T cell engager comprises HPN217 of Harpoon. In another embodiment, the T cell engager comprises a single flexible polypeptide comprising three binding domains.

In one embodiment, the bispecific antibody targeting BCMA is directed to BCMA and CD16A+. In one embodiment, CD16A+ (Fc receptor FcγRIIIa) is implicated in triggering lysis by NK cells, which in one embodiment, helps improve the efficacy of the treatment. In one embodiment, the bispecific antibody directed to BCMA and CD16A+ is AFM26 of Affimed N.V.

In one embodiment, the bispecific antibody targeting BCMA comprises a bispecific antibody directed to two different BCMA epitopes. In one embodiment, activating two key BCMA epitopes helps improve the efficacy of the treatment. In one embodiment, the bispecific antibody targeting BCMA directed to two key BCMA epitopes comprises LCAR-B38M of Nanjjing Legend Biotech Co. Ltd.

In one embodiment, the bispecific antibody targeting BCMA comprises a bispecific antibody directed to BCMA and CD3. In one embodiment, CD3 activates both cytotoxic T cells and T helper cells, which in one embodiment, improves the efficacy of the treatment. In one embodiment, the bispecific antibody directed to BCMA and CD3 comprises EM801 of Celgene Corp. In another embodiment, the bispecific antibody directed to BCMA and CD3 comprises PF-06863135 of Pfizer. In another embodiment, the bispecific antibody directed to BCMA and CD3 comprises TNB-381M, TNB-383M or TNB-383B of Teneobio Inc. In another embodiment, the bispecific antibody directed to BCMA and CD3 comprises JNJ-64007957 of Johnson & Johnson. In another embodiment, the bispecific antibody directed to BCMA and CD3 comprises AMG 420 (BI836909) of Amgen (BI). In one embodiment, the bispecific antibody comprises fully human VH domains.

In another embodiment, a tri-specific antibody targeting BCMA may be used in the compositions and methods of the present invention. In one embodiment, the tri-specific antibody is directed to human serum albumin (HSA), CD3 and BCMA. In one embodiment, the tri-specific antibody comprises HPN217 of Harpoon.

In one embodiment, BCMA is human BCMA having the amino acid sequence set forth below, or fragments thereof:

(SEQ ID NO: 1)
MLQMAGQCSQNEYFDSLLHACIPCQLRCSSNTPPLTCQRYCNASVTNSVK
GTNAILWTCLGLSLIISLAVFVLMFLLRKINSEPLKDEFKNTGSGLLGMA
NIDLEKSRTGDEIILPRGLEYTVEECTCEDCIKSKPKVDSDHCFPLPAME
EGATILVTTKTNDYCKSLPAALSATEIEKSISAR.

In another embodiment, the CAR T-cells of the compositions and methods as disclosed herein express a tumor antigen. In another embodiment, the tumor antigen is cleaved by gamma secretase. In one embodiment, the tumor antigen cleaved by gamma secretase comprises BCMA. In another embodiment, the tumor antigen cleaved by gamma secretase comprises a vascular endothelial growth factor receptor (VEGFR). In one embodiment, the VEGFR comprises VEGFR-1. In one embodiment, the VEGFR comprises VEGFR-2. In another embodiment, the tumor antigen cleaved by gamma secretase comprises CD44 or ErbB4.

In another embodiment, the CAR T-cells of the compositions and methods as disclosed herein target a substrate of γ-secretase. In one embodiment, the γ-secretase substrate comprises cluster of differentiation 43 (CD43), CD44, Colony-stimulating factor 1 receptor (CSF1R, CSF-1R), Delta1, E-cadherin, Ephrin B1, Ephrin B2, EphB2, ErbB4, growth hormone receptor (GHR), HLA-A2, insulin-like growth factor 1 receptor (IGF-1R), interferon alpha and beta receptor subunit 2 (IFNaR2), Interleukin 1 Receptor Type 2 (IL1R2), insulin receptor (IR), Ire1α, Jagged2, lipoprotein receptor-related protein (LRP), LRP1B, N-cadherin, Nectin-1α, Notch1, Notch2, Notch3, Notch4, or a combination thereof.

In another embodiment, the γ-secretase substrate comprises alcadein-α, alcadein-γ, Amyloid Beta Precursor Like Protein 1 (APLP1), APLP2, apolipoprotein E receptor 2 (ApoER2), amyloid precursor protein (APP), Chemokine (C-X-C motif) ligand 16 (CXCL16), CX3CL1, deleted in colorectal cancer (DCC), L1, LRP2, LRP6, neurotrophin receptor alike death domain protein (NRADD), p75-neurotrophin receptor (NTR), Polycystic Kidney and Hepatic Disease 1 (PKHD1), protocadherin (Pcdh) α4, Pcdh γ-C3, Protein Tyrosine Phosphatase (PTP)κ, PTPμ, PTP-leukocyte common antigen-related protein (LAR), Sortilin Related VPS10 Domain Containing Receptor 1b (SorCS1b), Sorting-related receptor with A-type repeats (SorLA), Sortilin, syndecan-3, tyrosinase, Tyrosinase-related protein 1 (TYRP1), TYRP2, vascular endothelial growth factor (VEGF)-1R, VEGF-R1, Voltage-gated sodium channel (VGSC) β2, Very Low Density Lipoprotein Receptor (VLDLR), or a combination thereof.

In another embodiment, the CAR T-cells of the compositions as disclosed herein express a tumor associated antigen (TAA). In one embodiment, the tumor associated antigen is: Mucin 1, cell surface associated (MUC1) or polymorphic epithelial mucin (PEM), Arginine-rich, mutated in early stage tumors (Armet), Heat Shock Protein 60 (HSP60), calnexin (CANX), methylenetetrahydrofolate dehydrogenase (NADP+ dependent) 2, methenyltetrahydrofolate cyclohydrolase (MTHFD2), fibroblast activation protein (FAP), matrix metallopeptidase (MMP6), B Melanoma Antigen-1 (BAGE-1), aberrant transcript of N-acetyl glucosaminyl transferase V (GnTV), Q5H943, Carcinoembryonic antigen (CEA), Pmel, Kallikrein-4, Mammaglobin-1, MART-1, GPR143-OA1, prostate specific antigen (PSA), TRP1, Tyrosinase, FGP-5, NEU proto-oncogene, Aft, MMP-2, prostate specific membrane antigen (PSMA), Telomerase-associated protein-2, Prostatic acid phosphatase (PAP), Uroplakin II or Proteinase 3.

In another embodiment, the CAR binds to CD19 or CD20 to target B cells in the case where one would like to destroy B cells as in leukemia. CD19 is a B cell lineage specific surface receptor whose broad expression, from pro-B cells to early plasma cells, makes it an attractive target for the immunotherapy of B cell malignancies. In another embodiment, the CAR binds to CD116. In another embodiment, the CAR binds to ROR1, CD22, or GD2. In another embodiment, the CAR binds to NY-ESO-1. In another embodiment, the CAR binds to MAGE family proteins. In another embodiment, the CAR binds to mesothelin (MSLN). In another embodiment, the CAR binds to c-erbB2. In another embodiment, the CAR binds to mutational antigens that are tumor specific, such as BRAFV600E mutations and BCR-ABL translocations. In another embodiment, the CAR binds to viral antigens which are tumor-specific, such as EBV in HD, HPV in cervical cancer, and polyomavirus in Merkel cancer. In another embodiment, the CAR T-cell binds to Her2/neu. In another embodiment, the CAR T-cell binds to EGFRvIII.

In one embodiment, the chimeric antigen receptor (CAR) T-cell binds the CD19 antigen. In another embodiment, the CAR T-cell binds the CD22 antigen. In another embodiment, the CAR T-cell binds to alpha folate receptor. In another embodiment, the CAR T-cell binds to carboxy-anhydrase-IX (CAIX). In another embodiment, the CAR T-cell binds to CD20. In another embodiment, the CAR T-cell binds to CD23. In another embodiment, the CAR T-cell binds to CD24. In another embodiment, the CAR T-cell binds to CD30. In another embodiment, the CAR T-cell binds to CD33. In another embodiment, the CAR T-cell binds to CD38. In another embodiment, the CAR T-cell binds to CD44v6. In another embodiment, the CAR T-cell binds to CD44v7/8. In another embodiment, the CAR T-cell binds to CD123. In another embodiment, the CAR T-cell binds to CD171. In another embodiment, the CAR T-cell binds to carcinoembryonic antigen (CEA). In another embodiment, the CAR T-cell binds to EGFRvIII. In another embodiment, the CAR T-cell binds to EGP-2. In another embodiment, the CAR T-cell binds to EGP-40. In another embodiment, the CAR T-cell binds to EphA2. In another embodiment, the CAR T-cell binds to Erb-B2. In another embodiment, the CAR T-cell binds to Erb-B 2,3,4. In another embodiment, the CAR T-cell binds to Erb-B3/4. In another embodiment, the CAR T-cell binds to FBP. In another embodiment, the CAR T-cell binds to fetal acetylcholine receptor. In another embodiment, the CAR T-cell binds to G_{D2}. In another embodiment, the CAR T-cell binds to G_{D3}. In another embodiment, the CAR T-cell binds to HER2. In another embodiment, the CAR T-cell binds to HMW-MAA. In another embodiment, the CAR T-cell binds to IL-11Ralpha. In another embodiment, the CAR T-cell binds to IL-13Ralpha1. In another embodiment, the CAR T-cell binds to KDR. In another embodiment, the CAR T-cell binds to kappa-light chain. In another embodiment, the CAR T-cell binds to Lewis Y. In another embodiment, the CAR T-cell binds to L1-cell adhesion molecule (CAM). In another embodiment, the CAR T-cell binds to MAGE-A1. In another embodiment, the CAR T-cell binds to mesothelin. In another embodiment, the CAR T-cell binds to CMV infected cells. In another embodiment, the CAR T-cell binds to MUC1. In another embodiment, the CAR T-cell binds to MUC16. In another embodiment, the CAR T-cell binds to NKG2D ligands. In another embodiment, the CAR T-cell binds to NY-ESO-1 (amino acids 157-165). In another embodiment, the CAR T-cell binds to oncofetal antigen (h5T4). In another embodiment, the CAR T-cell binds to PSCA. In another embodiment, the CAR T-cell binds to PSMA. In another embodiment, the CAR T-cell binds to ROR1. In another embodiment, the CAR T-cell binds to TAG-72. In another embodiment, the CAR T-cell binds to VEGF-R2 or other VEGF receptors. In another embodiment, the CAR T-cell binds to B7-H6. In another embodiment, the CAR T-cell binds to CA9. In another embodiment, the CAR T-cell binds to αᵥβ₆ integrin. In another embodiment, the CAR T-cell binds to 8H9. In another embodiment, the CAR T-cell binds to NCAM. In another embodiment, the CAR T-cell binds to fetal acetylcholine receptor.

In another embodiment, the chimeric antigen receptor (CAR) T-cell targets the CD19 antigen, and has a therapeutic effect on subjects with B-cell malignancies, ALL, Follicular lymphoma, CLL, and Lymphoma. In another embodiment, the CAR T-cell targets the CD22 antigen, and has a therapeutic effect on subjects with B-cell malignancies. In another embodiment, the CAR T-cell targets alpha folate receptor or folate receptor alpha (FR-α), and has a therapeutic effect on subjects with ovarian cancer or epithelial cancer. In another embodiment, the CAR T-cell targets CAIX or G250/CAIX, and has a therapeutic effect on subjects with renal cell carcinoma. In another embodiment, the CAR T-cell targets CD20, and has a therapeutic effect on subjects with Lymphomas, B-cell malignancies, B-cell lymphomas, Mantle cell lymphoma and, indolent B-cell lymphomas. In another embodiment, the CAR T-cell targets CD23, and has a therapeutic effect on subjects with CLL. In another embodiment, the CAR T-cell targets CD24, and has a therapeutic effect on subjects with pancreatic adenocarcinoma. In another embodiment, the CAR T-cell targets CD30, and has a therapeutic effect on subjects with Lymphomas or Hodgkin lymphoma. In another embodiment, the CAR T-cell targets CD33, and has a therapeutic effect on subjects with AML. In another embodiment, the CAR T-cell targets CD38, and has a therapeutic effect on subjects with Non-Hodgkin lymphoma. In another embodiment, the CAR T-cell targets CD44v6, and has a therapeutic effect on subjects with several malignancies. In another embodiment, the CAR T-cell targets CD44v7/8, and has a therapeutic effect on subjects with cervical carcinoma. In another embodiment, the CAR T-cell targets CD123, and has a therapeutic effect on subjects with myeloid malignancies. In another embodiment, the CAR T-cell targets CEA, and has a therapeutic effect on subjects with colorectal cancer, colorectal carcinoma, pancreatic adenocarcinoma, breast cancer. In another embodiment, the CAR T-cell targets EGFRvIII, and has a therapeutic effect on subjects with Glioblastoma. In another embodiment, the CAR T-cell targets Epidermal growth factor receptor (EGFR), and has a therapeutic effect on subjects with NSCLC, epithelial carcinoma, glioma. In another embodiment, the CAR T-cell targets EGP-2, and has a therapeutic effect on subjects with multiple malignancies. In another embodiment, the CAR T-cell targets EGP-40, and has a therapeutic effect on subjects with colorectal cancer. In another embodiment, the CAR T-cell targets EphA2, and has a therapeutic effect on subjects with Glioblastoma. In another embodiment, the CAR T-cell targets Erb-B2 or ErbB3/4, and has a therapeutic effect on subjects with Breast cancer and others, prostate cancer, colon cancer, various tumors. In another embodiment, the CAR T-cell targets Erb-B 2,3,4, and has a therapeutic effect on subjects with Breast cancer and others. In another embodiment, the CAR T-cell targets FBP, and has a therapeutic effect on subjects with Ovarian cancer. In another embodiment, the CAR T-cell targets fetal acetylcholine receptor, and has a therapeutic effect on subjects with Rhabdomyosarcoma. In another embodiment, the CAR T-cell targets G_{D2}, and has a therapeutic effect on subjects with Neuroblastoma, melanoma, or Ewing's sarcoma. In another embodiment, the CAR T-cell targets G_{D3}, and has a therapeutic effect on subjects with Melanoma. In another embodiment, the CAR T-cell targets HER2, and has a therapeutic effect on subjects with medulloblastoma, pancreatic adenocarcinoma, Glioblastoma, Osteosarcoma, breast cancer, colon cancer, or ovarian cancer. In another embodiment, the CAR T-cell targets HMW-MAA, and has a therapeutic effect on subjects with Melanoma. In another embodiment, the CAR T-cell targets IL-I1Ralpha, and has a therapeutic effect on subjects with Osteosarcoma. In another embodiment, the CAR T-cell targets IL-13Ralpha1, and has a therapeutic effect on subjects with Glioma, Glioblastoma, or medulloblastoma. In another embodiment, the CAR T-cell targets IL-13 receptor alpha2, and has a therapeutic effect on subjects with glioma or other malignancies. In another embodiment, the CAR T-cell targets KDR, and has a therapeutic effect on subjects with tumors by targeting tumor neovasculature. In another embodiment, the CAR T-cell targets kappa-light chain, and has a therapeutic effect on subjects with B-cell malignancies (B-NHL, CLL). In another embodiment, the CAR T-cell targets Lewis Y, and has a therapeutic effect on subjects with various carcinomas or epithelial-derived tumors. In another embodiment, the CAR T-cell targets L1-cell adhesion molecule (L1-CAM), and has a therapeutic effect on subjects with neuroblastoma, melanoma, ovarian adenocarcinoma. In another embodiment, the CAR T-cell targets MAGE-A1 or HLA-A1 MAGE A1, and has a therapeutic effect on subjects with Melanoma. In another embodiment, the CAR T-cell targets mesothelin, and has a therapeutic effect on subjects with Mesothelioma, ovarian cancer, pancreatic adenocarcinoma. In another embodiment, the CAR T-cell targets CMV infected cells, and has a therapeutic effect on subjects with CMV. In another embodiment, the CAR T-cell targets MUC1, and has a therapeutic effect on subjects with breast or ovarian cancer or on subjects with seminal vesicle cancer. In another embodiment, the CAR T-cell targets Cancer antigen 125 (also known as MUC16) (CA125), and has a therapeutic effect on subjects with ovarian cancer, including epithelial ovarian cancers. In another embodiment, the CAR T-cell targets NKG2D ligands, and has a therapeutic effect on subjects with myeloma, ovarian, and other tumors. In another embodiment, the CAR T-cell targets Cancer/testis antigen 1B (also known as NY-ESO-1) (CTAG1B), and has a therapeutic effect on subjects with Melanoma and ovarian cancer. In another embodiment, the CAR T-cell targets NY-ESO-1 (157-165) or HLA-A2 NY-ESO-1, and has a therapeutic effect on subjects with multiple myeloma. In another embodiment, the CAR T-cell targets oncofetal antigen (h5T4), and has a therapeutic effect on subjects with various tumors. In another embodiment, the CAR T-cell targets PSCA, and has a therapeutic effect on subjects with prostate carcinoma. In another embodiment, the CAR T-cell targets PSMA, and has a therapeutic effect on subjects with prostate cancer/tumor vasculature. In another embodiment, the CAR T-cell targets ROR1, and has a therapeutic effect on subjects with B-CLL and mantle cell lymphoma. In another embodiment, the CAR T-cell targets TAG-72, and has a therapeutic effect on subjects with adenocarcinomas or gastrointestinal cancers. In another embodiment, the CAR T-cell targets VEGF-R2 or other VEGF receptors, and has a therapeutic effect on subjects with tumors by targeting tumor neovasculature. In another embodiment, the CAR T-cell targets CA9, and has a therapeutic effect on subjects with renal cell carcinoma. In another embodiment, the CAR T-cell targets CD171, and has a therapeutic effect on subjects with renal neuroblastoma. In another embodiment, the CAR T-cell targets NCAM, and has a therapeutic effect on subjects with neuroblastoma. In another embodiment, the CAR T-cell targets fetal acetylcholine receptor, and has a therapeutic effect on subjects with rhabdomyosarcoma. In another embodiment, the CAR T-cell targets Glypican-3 (GPC3), and has a therapeutic effect on subjects with Hepatocellular carcinoma. In another embodiment, the CAR T-cell targets Cluster of differentiation 133 (also known as prominin-1) (CD133), and has a therapeutic effect on subjects with Glioblastoma, cholangiocarcinoma (CCA). In another embodiment, the CAR T-cell targets Fibroblast activation protein (FAP), and has a therapeutic effect on subjects with Malignant pleural mesothelioma (MPM).

In another embodiment, the CAR binds to one of the target antigens listed in Table 1 of Sadelain et al. (Cancer Discov. 2013 April; 3(4): 388-398), which is incorporated by reference herein in its entirety. In another embodiment, CAR T-cells express carbohydrate or glycolipid structures.

In one embodiment the CAR binds to an angiogenic factor, thereby targeting tumor vasculature. In one embodiment, the angiogenic factor is VEGFR-2. In another embodiment, the angiogenic factor is VEGFR-1. In another embodiment, the angiogenic factor is endoglin. In another embodiment, an angiogenic factor of the present invention is Angiogenin; Angiopoietin-1; Del-1; Fibroblast growth factors: acidic (aFGF) and basic (bFGF); Follistatin; Granulocyte colony-stimulating factor (G-CSF); Hepatocyte growth factor (HGF)/scatter factor (SF); Interleukin-8 (IL-8); Leptin; Midkine; Placental growth factor; Platelet-derived endothelial cell growth factor (PD-ECGF); Platelet-derived growth factor-BB (PDGF-BB); Pleiotrophin (PTN); Progranulin; Proliferin; Transforming growth factor-alpha (TGF-alpha); Transforming growth factor-beta (TGF-beta); Tumor necrosis factor-alpha (TNF-alpha); Vascular endothelial growth factor (VEGF)/vascular permeability factor (VPF). In another embodiment, an angiogenic factor is an angiogenic protein. In one embodiment, a growth factor is an angiogenic protein. In one embodiment, an angiogenic protein for use in the compositions and methods of the present invention is Fibroblast growth factors (FGF); VEGF; VEGFR and Neuropilin 1 (NRP-1); Angiopoietin 1 (Ang1) and Tie2; Platelet-derived growth factor (PDGF; BB-homodimer) and PDGFR; Transforming growth factor-beta (TGF-β), endoglin and TGF-β receptors; monocyte chemotactic protein-1 (MCP-1); Integrins αVβ3, αVβ5 and α5β1; VE-cadherin and CD31; ephrin; plasminogen activators; plasminogen activator inhibitor-1; Nitric oxide synthase (NOS) and COX-2; AC133; or Id1/Id3. In one embodiment, an angiogenic protein for use in the compositions and methods of the present invention is an angiopoietin, which in one embodiment, is Angiopoietin 1, Angiopoietin 3, Angiopoietin 4 or Angiopoietin 6. In one embodiment, endoglin is also known as CD105; EDG; HHT1; ORW; or ORW1. In one embodiment, endoglin is a TGFbeta co-receptor.

In another embodiment, the CAR T-cells express an antigen associated with an infectious agent. In one embodiment, the infectious agent is Mycobacterium ruberculosis. In one embodiment, said Mycobacterium tuberculosis associated antigen is: Antigen 85B, Lipoprotein IpqH, ATP dependent helicase putative, uncharacterized protein Rv0476/MTO4941 precursor or uncharacterized protein Rv1334/MIT1376 precursor.

In another embodiment, the CAR binds to an antibody. In one embodiment, the CAR T-cell is an “antibody-coupled T-cell receptor” (ACTR). According to this embodiment, the CAR T-cell is a universal CAR T-cell. In another embodiment, the CAR T-cell having an antibody receptor is administered before, after, or at the same time as the antibody is administered and then binds to the antibody, bringing the T-cell in close proximity to the tumor or cancer. In another embodiment, the antibody is directed against a tumor cell antigen. In another embodiment, the antibody is directed against CD20. In another embodiment, the antibody is rituximab.

In another embodiment, the antibody is Trastuzumab (Herceptin; Genentech): humanized IgG1, which is directed against ERBB2. In another embodiment, the antibody is Bevacizumab (Avastin; Genentech/Roche): humanized IgG1, which is directed against VEGF. In another embodiment, the antibody is Cetuximab (Erbitux; Bristol-Myers Squibb): chimeric human-murine IgG1, which is directed against EGFR. In another embodiment, the antibody is Panitumumab (Vectibix; Amgen): human IgG2, which is directed against EGFR. In another embodiment, the antibody is Ipilimumab (Yervoy; Bristol-Myers Squibb): IgG1, which is directed against CTLA4.

In another embodiment, the antibody is Alemtuzumab (Campath; Genzyme): humanized IgG1, which is directed against CD52. In another embodiment, the antibody is Ofatumumab (Arzerra; Genmab): human IgG1, which is directed against CD20. In another embodiment, the antibody is Gemtuzumab ozogamicin (Mylotarg; Wyeth): humanized IgG4, which is directed against CD33. In another embodiment, the antibody is Brentuximab vedotin (Adcetris; Seattle Genetics): chimeric IgG1, which is directed against CD30. In another embodiment, the antibody is 90Y-labelled ibritumomab tiuxetan (Zevalin; IDEC Pharmaceuticals): murine IgG1, which is directed against CD20. In another embodiment, the antibody is 131I-labelled tositumomab (Bexxar; GlaxoSmithKline): murine IgG2, which is directed against CD20.

In another embodiment, the antibody is Ramucirumab, which is directed against vascular endothelial growth factor receptor-2 (VEGFR-2). In another embodiment, the antibody is ramucirumab (Cyramza Injection, Eli Lilly and Company), blinatumomab (BLINCYTO, Amgen Inc.), pembrolizumab (KEYTRUDA, Merck Sharp & Dohme Corp.), obinutuzumab (GAZYVA, Genentech, Inc.; previously known as GA101), pertuzumab injection (PERJETA, Genentech, Inc.), or denosumab (Xgeva, Amgen Inc.). In another embodiment, the antibody is Basiliximab (Simulect; Novartis). In another embodiment, the antibody is Daclizumab (Zenapax; Roche).

In another embodiment, the antibody to which the CAR T-cell is coupled is directed to a tumor or cancer antigen or a portion thereof, that is described herein and/or that is known in the art. In another embodiment, the antibody to which the CAR T-cell is couples is directed to a tumor-associated antigen. In another embodiment, the antibody to which the CAR T-cell is couples is directed to a tumor-associated antigen or a portion thereof that is an angiogenic factor.

A skilled artisan would appreciate that a genetically modified TCR may be engineered to recognize any of the antigens described above to which a CAR binds. In one embodiment, a TCR T-cell binds to an antigen described above as a CAR T-cell binding target. In another embodiment, a TCR recognizes any antigen disclosed herein. In another embodiment, the antigen to which the TCR recognizes is a tumor or cancer antigen or a portion thereof, that is described herein and/or that is known in the art. In another embodiment, the TCR recognizes a tumor-associated antigen. In another embodiment, the TCR recognizes a tumor-associated antigen or a portion thereof that is an angiogenic factor.

In one embodiment, compositions and methods as described herein incorporate immune checkpoint blockade, which in one embodiment, comprises PD-1/PD-L1 blockade. In one embodiment, PD-1/PD-L1 blockade comprises administration of anti-PD-1, anti-PD-L1, anti-CTLA-4 antibodies, or a combination thereof.

In another embodiment, CAR-T cells are engineered to produce molecules that block PD-1/PD-L1. In one embodiment, CAR-T cells are engineered to secrete anti-PD-1/PD-L1 or anti-CTLA-4 antibodies. In another embodiment, CAR-T cells are engineered to express a PD-1 dominant negative receptor (DNR) or a PD-1:CD28 chimeric switch-receptor (CSR). In another embodiment, CAR-T cells are PD-1 deficient. In another embodiment, PD-1 expression is downregulated by a PD-1 shRNA lentiviral cassette. In another embodiment, CAR-T cells are engineered with knockouts of TCR, Beta2-Microglobulin and/or Human Leukocyte Antigen (HLA). In one embodiment, ablating the endogenous TCR followed by selective depletion of native TCR+ cells allows for the generation of highly potent, tumor specific CAR-T cells lacking any capacity to target non-CAR antigens. In another embodiment, CAR-T cells are engineered with to be expressed under the TRAC gene promoter, which in one embodiment, leads to uniform levels of CAR expression, averts tonic CAR-signaling, establishes effective internalization and re-expression of CAR following single or repeated exposure to antigen, and delays effector T cell differentiation and exhaustion.

In another embodiment, immune checkpoint blockade comprises blockade of LAG-3, TIM-3, CTLA-4, SHP-1, adenosine 2A receptor (A2AR), or a combination thereof, using any of the techniques described for PD-1/PD-L1 blockade as described hereinabove.

All of these are to be considered as embodiments of the present invention.

### Combined Treatments

In treating cancer, a combination of chemotherapeutic agents and/or other treatments (e.g., radiation therapy) is often advantageous. An additional agent may have the same or different mechanism of action than the primary therapeutic agents. For example, drug combinations may be employed wherein the two or more drugs being administered act in different manners or in different phases of the cell cycle, and/or where the two or more drugs have nonoverlapping toxicities or side effects, and/or where the drugs being combined each has a demonstrated efficacy in treating the particular disease state manifested by the patient.

In one embodiment, a method is provided for treating cancer comprising administering to a mammal in need thereof a composition as described herein and administering one or more additional anti-cancer agents.

In one embodiment, the phrase “additional anti-cancer agent” refers to a drug selected from any one or more of the following: alkylating agents (including nitrogen mustards, methanesulphonate, busulphan, alkyl sulfonates, nitrosoureas, ethylenimine derivatives, and triazenes, or a combination thereof); anti-angiogenics (including matrix metalloproteinase inhibitors); antimetabolites (including adenosine deaminase inhibitors, folic acid antagonists, purine analogues, and pyrimidine analogues); antibiotics or antibodies (including monoclonal antibodies, CTLA-4 antibodies, anthracyclines); aromatase inhibitors; cell-cycle response modifiers; enzymes; farnesyl-protein transferase inhibitors; hormonal and antihormonal agents and steroids (including synthetic analogs, glucocorticoids, estrogens/anti-estrogens [e.g., SERMs], androgens/anti-androgens, progestins, progesterone receptor agonists, and luteinizing hormone-releasing [LHRH] agonists and antagonists); insulin-like growth factor (IGF)/insulin-like growth factor receptor (IGFR) system modulators (including IGFR1 inhibitors); integrin-signaling inhibitors; kinase inhibitors (including multi-kinase inhibitors and/or inhibitors of Src kinase or Src/ab1, cyclin dependent kinase [CDK] inhibitors, panHer, Her-1 and Her-2 antibodies, VEGF inhibitors, including anti-VEGF antibodies, EGFR inhibitors, PARP (poly ADP-ribose polymerase) inhibitors, mitogen-activated protein [MAP] inhibitors, MET inhibitors, MEK inhibitors, Aurora kinase inhibitors, PDGF inhibitors, and other tyrosine kinase inhibitors or serine/threonine kinase inhibitors; microtubule-disruptor agents, such as ecteinascidins or their analogs and derivatives; microtubule-stabilizing agents such as taxanes, platinum-based antineoplastic drugs (platins) such as cisplatin, carboplatin, oxaliplatin, nedaplatin, triplatin tetranitrate, phenanthriplatin, picoplatin and satraplatin, and the naturally-occurring epothilones and their synthetic and semi-synthetic analogs; microtubule-binding, destabilizing agents (including vinca alkaloids); topoisomerase inhibitors; prenyl-protein transferase inhibitors; platinum coordination complexes; signal transduction inhibitors; and other agents used as anti-cancer and cytotoxic agents such as biological response modifiers, growth factors, and immune modulators.

In another embodiment, the present invention provides a composition comprising one or more compounds represented by the structure of Formula (I) as described herein in combination with idecabtagene vicleucel. In another embodiment, the present invention provides a composition comprising one or more compounds represented by the structure of Formula (I) as described herein in combination with belantamab mafodotin. In another embodiment, the present invention provides a composition comprising one or more compounds represented by the structure of Formula (I) as described herein in combination with HDP-101 (Heidelberg Pharma AG). In another embodiment, the present invention provides a composition comprising one or more compounds represented by the structure of Formula (I) as described herein in combination with gene therapy to target BCMA for B-Cell Leukemia or Lymphoma, which in one embodiment, is from Max Delbruck Center for Molecular Medicine and, in another embodiment, is from Third Military Medical University Hospital One.

In another embodiment, the present invention provides a composition comprising one or more compounds represented by the structure of Formula (I) as described herein in combination with ABL-201. In another embodiment, the present invention provides a composition comprising one or more compounds represented by the structure of Formula (I) as described herein in combination with ACTR-087+SEA-BCMA. In another embodiment, the present invention provides a composition comprising one or more compounds represented by the structure of Formula (I) as described herein in combination with AFM-26. In another embodiment, the present invention provides a composition comprising one or more compounds represented by the structure of Formula (I) as described herein in combination with ALLO-715. In another embodiment, the present invention provides a composition comprising one or more compounds represented by the structure of Formula (I) as described herein in combination with AMG-224. In another embodiment, the present invention provides a composition comprising one or more compounds represented by the structure of Formula (I) as described herein in combination with AMG-420. In another embodiment, the present invention provides a composition comprising one or more compounds represented by the structure of Formula (I) as described herein in combination with AMG-701. In another embodiment, the present invention provides a composition comprising one or more compounds represented by the structure of Formula (I) as described herein in combination with AUTO-2. In another embodiment, the present invention provides a composition comprising one or more compounds represented by the structure of Formula (I) as described herein in combination with bb-21217. In another embodiment, the present invention provides a composition comprising one or more compounds represented by the structure of Formula (I) as described herein in combination with belantamab mafodotin. In another embodiment, the present invention provides a composition comprising one or more compounds represented by the structure of Formula (I) as described herein in combination with CC-93269. In another embodiment, the present invention provides a composition comprising one or more compounds represented by the structure of Formula (I) as described herein in combination with CC-99712. In another embodiment, the present invention provides a composition comprising one or more compounds represented by the structure of Formula (I) as described herein in combination with CCAR-088. In another embodiment, the present invention provides a composition comprising one or more compounds represented by the structure of Formula (I) as described herein in combination with CMD-505. In another embodiment, the present invention provides a composition comprising one or more compounds represented by the structure of Formula (I) as described herein in combination with CRC-03. In another embodiment, the present invention provides a composition comprising one or more compounds represented by the structure of Formula (I) as described herein in combination with CT-053. In another embodiment, the present invention provides a composition comprising one or more compounds represented by the structure of Formula (I) as described herein in combination with CT-103A. In another embodiment, the present invention provides a composition comprising one or more compounds represented by the structure of Formula (I) as described herein in combination with CTX-120. In another embodiment, the present invention provides a composition comprising one or more compounds represented by the structure of Formula (I) as described herein in combination with CTX-4419. In another embodiment, the present invention provides a composition comprising one or more compounds represented by the structure of Formula (I) as described herein in combination with CTX-8573. In another embodiment, the present invention provides a composition comprising one or more compounds represented by the structure of Formula (I) as described herein in combination with CYAD-211. In another embodiment, the present invention provides a composition comprising one or more compounds represented by the structure of Formula (I) as described herein in combination with Descartes-08. In another embodiment, the present invention provides a composition comprising one or more compounds represented by the structure of Formula (I) as described herein in combination with EM-801. In another embodiment, the present invention provides a composition comprising one or more compounds represented by the structure of Formula (I) as described herein in combination with FCARH-143. In another embodiment, the present invention provides a composition comprising one or more compounds represented by the structure of Formula (I) as described herein in combination with FT-576. In another embodiment, the present invention provides a composition comprising one or more compounds represented by the structure of Formula (I) as described herein in combination with HDP-101. In another embodiment, the present invention provides a composition comprising one or more compounds represented by the structure of Formula (I) as described herein in combination with HPN-217. In another embodiment, the present invention provides a composition comprising one or more compounds represented by the structure of Formula (I) as described herein in combination with HRAIN-002. In another embodiment, the present invention provides a composition comprising one or more compounds represented by the structure of Formula (I) as described herein in combination with HY-015. In another embodiment, the present invention provides a composition comprising one or more compounds represented by the structure of Formula (I) as described herein in combination with ICTCAR-032. In another embodiment, the present invention provides a composition comprising one or more compounds represented by the structure of Formula (I) as described herein in combination with idecabtagene vicleucel. In another embodiment, the present invention provides a composition comprising one or more compounds represented by the structure of Formula (I) as described herein in combination with IM-21. In another embodiment, the present invention provides a composition comprising one or more compounds represented by the structure of Formula (I) as described herein in combination with JNJ-7957. In another embodiment, the present invention provides a composition comprising one or more compounds represented by the structure of Formula (I) as described herein in combination with LCARB-38M. In another embodiment, the present invention provides a composition comprising one or more compounds represented by the structure of Formula (I) as described herein in combination with MCARH-171. In another embodiment, the present invention provides a composition comprising one or more compounds represented by the structure of Formula (I) as described herein in combination with MEDI-2228. In another embodiment, the present invention provides a composition comprising one or more compounds represented by the structure of Formula (I) as described herein in combination with orvacabtagene autoleucel. In another embodiment, the present invention provides a composition comprising one or more compounds represented by the structure of Formula (I) as described herein in combination with PBCAR-269A. In another embodiment, the present invention provides a composition comprising one or more compounds represented by the structure of Formula (I) as described herein in combination with PBCMA-101. In another embodiment, the present invention provides a composition comprising one or more compounds represented by the structure of Formula (I) as described herein in combination with PBCMAALL-O1. In another embodiment, the present invention provides a composition comprising one or more compounds represented by the structure of Formula (I) as described herein in combination with PF-06863135. In another embodiment, the present invention provides a composition comprising one or more compounds represented by the structure of Formula (I) as described herein in combination with REGN-5458. In another embodiment, the present invention provides a composition comprising one or more compounds represented by the structure of Formula (I) as described herein in combination with SEA-BCMA. In another embodiment, the present invention provides a composition comprising one or more compounds represented by the structure of Formula (I) as described herein in combination with TNB-383B.

In another embodiment, the present invention provides a composition comprising one or more compounds represented by the structure of Formula (I) as described herein in combination with Bispecific Monoclonal Antibodies to Target PDL1 and BCMA for Multiple Myeloma. In another embodiment, the present invention provides a composition comprising one or more compounds represented by the structure of Formula (I) as described herein in combination with Bispecific Monoclonal Antibody To Target BCMA and CD3 for Multiple Myeloma. In another embodiment, the present invention provides a composition comprising one or more compounds represented by the structure of Formula (I) as described herein in combination with Bispecific Monoclonal Antibody to Target CD3 and BCMA for Multiple Myeloma. In another embodiment, the present invention provides a composition comprising one or more compounds represented by the structure of Formula (I) as described herein in combination with Bispecific Monoclonal Antibody to Target CD3 and BCMA for Oncology. In another embodiment, the present invention provides a composition comprising one or more compounds represented by the structure of Formula (I) as described herein in combination with Gene Therapy to Target BCMA and CD19 for Relapsed and Refractory Multiple Myeloma and POMES syndrome. In another embodiment, the present invention provides a composition comprising one or more compounds represented by the structure of Formula (I) as described herein in combination with Gene Therapy to Target BCMA and CD38 for Multiple Myeloma. In another embodiment, the present invention provides a composition comprising one or more compounds represented by the structure of Formula (I) as described herein in combination with Gene Therapy to Target BCMA and CD38 for Relapsed and Refractory Multiple Myeloma. In another embodiment, the present invention provides a composition comprising one or more compounds represented by the structure of Formula (I) as described herein in combination with Gene Therapy to Target BCMA and CS1 for Multiple Myeloma. In another embodiment, the present invention provides a composition comprising one or more compounds represented by the structure of Formula (I) as described herein in combination with Gene Therapy to Target BCMA for B-Cell Leukemia and Lymphoma. In another embodiment, the present invention provides a composition comprising one or more compounds represented by the structure of Formula (I) as described herein in combination with Gene Therapy to Target BCMA for Multiple Myeloma. In another embodiment, the present invention provides a composition comprising one or more compounds represented by the structure of Formula (I) as described herein in combination with Gene Therapy to Target BCMA for Recurrent and Refractory Multiple Myeloma. In another embodiment, the present invention provides a composition comprising one or more compounds represented by the structure of Formula (I) as described herein in combination with Gene Therapy to Target BCMA for Refractory Multiple Myeloma. In another embodiment, the present invention provides a composition comprising one or more compounds represented by the structure of Formula (I) as described herein in combination with Gene Therapy to Target BCMA for Relapsed and Refractory Multiple Myeloma. In another embodiment, the present invention provides a composition comprising one or more compounds represented by the structure of Formula (I) as described herein in combination with Gene Therapy to Target CD138 and CD269 for Relapsed and Refractory Multiple Myeloma. In another embodiment, the present invention provides a composition comprising one or more compounds represented by the structure of Formula (I) as described herein in combination with Gene Therapy to Target CD19 and CD269 for Multiple Myeloma. In another embodiment, the present invention provides a composition comprising one or more compounds represented by the structure of Formula (I) as described herein in combination with Gene Therapy to Target CD269 for Multiple Myeloma. In another embodiment, the present invention provides a composition comprising one or more compounds represented by the structure of Formula (I) as described herein in combination with Gene Therapy to Target CD38 and CD269 for Multiple Myeloma. In another embodiment, the present invention provides a composition comprising one or more compounds represented by the structure of Formula (I) as described herein in combination with Gene Therapy to Target CD38 and TNFRSF17 for Relapsed and Refractory Multiple Myeloma. In another embodiment, the present invention provides a composition comprising one or more compounds represented by the structure of Formula (I) as described herein in combination with Gene Therapy to Target Cells Expressing BCMA for Multiple Myeloma. In another embodiment, the present invention provides a composition comprising one or more compounds represented by the structure of Formula (I) as described herein in combination with Gene Therapy to Target TNFRSF17 for B-Cell Non-Hodgkin Lymphoma and Multiple Myeloma. In another embodiment, the present invention provides a composition comprising one or more compounds represented by the structure of Formula (I) as described herein in combination with Gene Therapy to Target TNFRSF17 for Multiple Myeloma. In another embodiment, the present invention provides a composition comprising one or more compounds represented by the structure of Formula (I) as described herein in combination with Monoclonal Antibodies to Target BCMA for Multiple Myeloma and Autoimmune Diseases. In another embodiment, the present invention provides a composition comprising one or more compounds represented by the structure of Formula (I) as described herein in combination with Monoclonal Antibodies to Target BCMA for Multiple Myeloma and Autoimmune Diseases.

In another embodiment, the present invention provides a composition comprising one or more compounds represented by the structure of Formula (I) as described herein in combination with an inhibitor of mammalian target of rapamycin (mTOR). In one embodiment, the mTOR inhibitor comprises Everolimus. In another embodiment, the mTOR inhibitor comprises sirolimus (rapamycin). In another embodiment, the mTOR inhibitor comprises temsirolimus.

In another embodiment, the mTOR inhibitor comprises a dual mammalian target of rapamycin/phosphoinositide 3-kinase inhibitor, which in one embodiment, comprises NVP-BEZ235 (dactolisib), GSK2126458, XL765, or a combination thereof.

In another embodiment, the mTOR inhibitor comprises a second generation mTOR inhibitor, which, in one embodiment, comprises AZD8055, INK128/MLN0128, OSI027, or a combination thereof.

In another embodiment, the mTOR inhibitor comprises a third generation mTOR inhibitor, which, in one embodiment, comprises RapaLinks.

In another embodiment, the present invention provides a composition comprising one or more compounds represented by the structure of Formula (I) as described herein in combination with an mTOR inhibitor and a chemotherapeutic drug. In one embodiment, the mTOR inhibitor comprises everolimus. In one embodiment, the chemotherapeutic drug comprises cisplatin.

In another embodiment, the present invention provides a composition comprising one or more compounds represented by the structure of Formula (I) as described herein in combination with bisphosphonates.

In another embodiment, the present invention provides a composition comprising one or more compounds represented by the structure of Formula (I) as described herein in combination with cancer growth blockers.

In another embodiment, the present invention provides a composition comprising one or more compounds represented by the structure of Formula (I) as described herein in combination with proteasome inhibitors.

In another embodiment, the present invention provides a composition comprising one or more compounds represented by the structure of Formula (I) as described herein in combination with one or more interferons.

In another embodiment, the present invention provides a composition comprising one or more compounds represented by the structure of Formula (I) as described herein in combination with one or more interleukins.

In another embodiment, the present invention provides a composition comprising one or more compounds represented by the structure of Formula (I) as described herein in combination with an alkylating drug. In one embodiment, the alkylating drug comprises Procarbazine (MATULANE®), Dacarbazine (DTIC), Altretamine (HEXALEN®), or a combination thereof.

In another embodiment, the present invention provides a composition comprising one or more compounds represented by the structure of Formula (I) as described herein in combination with an antimetabolite. In one embodiment, the antimetabolite comprises an antifolic acid compound (Methotrexate), an amino acid antagonists (Azaserine), or a combination thereof.

In another embodiment, the present invention provides a composition comprising one or more compounds represented by the structure of Formula (I) as described herein in combination with a purine antagonist. In one embodiment, the purine antagonist comprises Mercaptopurine (6-MP), Thioguanine (6-TG), Fludarabine Phosphate, Cladribine (LEUSTATIN®), Pentostatin (NIPENT™) or a combination thereof.

In another embodiment, the present invention provides a composition comprising one or more compounds represented by the structure of Formula (I) as described herein in combination with a pyrimidine antagonist. In one embodiment, the pyrimidine antagonist comprises Fluorouracil (5-FU), Cytarabine (ARA-C), Azacitidine, or a combination thereof.

In another embodiment, the present invention provides a composition comprising one or more compounds represented by the structure of Formula (I) as described herein in combination with a plant alkaloid. In one embodiment, the pyrimidine antagonist comprises Vinblastine (VELBAN®), Vincristine (ONCOVIN®), Etoposide (VP-16, VEPE-SID®), Teniposide (VUMON®), Topotecan (HYCAMTIN®), Irinotecan (CAMPTOSAR®), Paclitaxel (TAXOL®), Docetaxel (TAXOTERE®), or a combination thereof.

In another embodiment, the present invention provides a composition comprising one or more compounds represented by the structure of Formula (I) as described herein in combination with FOLFIRI, wherein in one embodiment FOLFIRI comprises folinic acid (leucovorin), fluorouracil (5-FU) and irinotecan (CAMPTOSAR®). In another embodiment, the present invention provides a composition comprising one or more compounds represented by the structure of Formula (I) as described herein in combination with folinic acid (leucovorin), fluorouracil (5-FU), irinotecan (CAMPTOSAR®), or a combination thereof.

In another embodiment, the present invention provides a composition comprising one or more compounds represented by the structure of Formula (I) as described herein in combination with an antibiotic. In one embodiment, the antibiotic comprises Anthracyclines, Doxorubicin (ADRIAMYCIN, RUBEX, DOXIL®), Daunorubicin (DAUNOXOME®), Dactinomycin (COSMEGEN®), Idarubincin (IDAMYCIN®), Plicamycin (MITHRAMYCIN®), Mitomycin (MUTAMYCIN®), Bleomycin (BLENOXANE®), or a combination thereof.

In another embodiment, the present invention provides a composition comprising one or more compounds represented by the structure of Formula (I) as described herein in combination with an immunotherapeutic. In one embodiment, the immunotherapeutic comprises a monoclonal antibody. In one embodiment, the monoclonal antibody comprises an anti-PD-1 antibody, which in one embodiment comprises nivolumab.

In another embodiment, the monoclonal antibody comprises alemtuzumab (CAMPATH®), trastuzumab (HERCEPTIN®), Bevacizumab (AVASTIN®), Cetuximab (ERBITUX®), or a combination thereof. In another embodiment, the monocolonal antibody comprises a radiolabeled antibody, which, in one embodiment, comprises britumomab, tiuxetan (ZEVALIN®), or a combination thereof. In another embodiment, the monocolonal antibody comprises a chemolabeled antibody, which in one embodiment comprises Brentuximab vedotin (ADCETRIS®), Ado-trastuzumab emtansine (KADCYLA®, also called TDM-1), denileukin diftitox (ONTAK®), or a combination thereof, In another embodiment, the monocolonal antibody comprises a bispecific antibody, which in one embodiment, comprises blinatumomab (BLINCYTO®).

In another embodiment, the present invention provides a composition comprising one or more compounds represented by the structure of Formula (I) as described herein in combination with a hormonal agent. In one embodiment, the hormonal agent comprises Tamoxifen (NOLVADEX®), Flutamide (EULEXIN®), Gonadotropin-Releasing Hormone Agonists, (Leuprolide and Goserelin (ZOLADEX®)), Aromatase Inhibitors, Aminoglutethimide, Anastrozole (ARIMIDEX®), or a combination thereof.

In another embodiment, the present invention provides a composition comprising one or more compounds represented by the structure of Formula (I) as described herein in combination with Amsacrine, Hydroxyurea (Hydrea), Asparaginase (EL-SPAR®), Mitoxantrone (NOVANTRONE®), Mitotane, Retinoic Acid Derivatives, Bone Marrow Growth Factors, Amifostine, or a combination thereof.

In another embodiment, the present invention provides a composition comprising one or more compounds represented by the structure of Formula (I) as described herein in combination with an agent that inhibits one or more cancer stem cell pathways. In one embodiment, such agent comprises an inhibitor of Hedgehog, WNT, BMP, or a combination thereof.

In another embodiment, the present invention provides a composition comprising one or more compounds represented by the structure of Formula (I) as described herein in combination with any one or more of the following: REVLIMID® (lenalidomide), AVASTIN® (Bevacizumab), HERCEPTIN® (trastuzumab), RITUXAN® (rituximab), OPDIVO® (nivolumab), GLEEVEC® (imatinib mesylate), IMBRUVICA® (ibrutinib), VELCADE® (bortezomib), ZYTIGA® (abiraterone acetate), XTANDI® (enzalutamide), ALIMTA® (Pemetrexed), GARDASIL®, IBRANCE® (palbociclib), PERJETA® (pertuzumab), TASIGNA® (nilotinib), XGEVA® (Denosumab), AFINITOR® (everolimus), JAKAFI® (ruxolitinib), TARCEVA® (erlotinib), KEYTRUDA® (pembrolizumab), SUTENT® (sunitinib malate)|, YERVOY® (ipilimumab), NEXAVAR® (sorafenib), ZOLADEX (goserelin acetate), ERBITUX®, DARZALEX® (daratumumab), XELODA® (capecitabine), GAZYVA® (obinutuzumab), VENCLEXTA® (venetoclax), and TECENTRIQ® (atezolizumab).

In another embodiment, the present invention provides a composition comprising one or more compounds represented by the structure of Formula (I) as described herein in combination with any one or more of the following: abemaciclib, epacadostat, apalutamide, Carfilzomib, Crizotinib (PF-02341066), GDC-0449 (vismodegib), ONCOVEX^{GM-CSF}, PLX4032 (RG7204), Ponatinib, SGN-35 (brentuximab vedotin), Tivozanib (AV-951), T-DM1 (Trastuzumab-DM1), and XL184 (cabozantinib).

Accordingly, the compositions of the present invention may be administered in combination with other anti-cancer treatments useful in the treatment of cancer or other proliferative diseases. The invention herein further comprises use of the compositions of the present invention in preparing medicaments for the treatment of cancer, and/or it comprises the packaging of the compositions of the present invention together with instructions that the compositions be used in combination with other anti-cancer or cytotoxic agents and treatments for the treatment of cancer.

In one embodiment, a method is provided for treating cancer comprising administering to a mammal in need thereof a combination of a compound of Formula (I) and CAR-T therapy; administering dasatinib; and optionally, one or more additional anti-cancer agents.

In one embodiment, a method is provided for treating cancer comprising administering to a mammal in need thereof a combination of a compound of Formula (I) and CAR-T therapy; administering paclitaxel; and optionally, one or more additional anti-cancer agents.

In one embodiment, a method is provided for treating cancer comprising administering to a mammal in need thereof a combination of a compound of Formula (I) and CAR-T therapy; administering Tamoxifen; and optionally, one or more additional anti-cancer agents.

In one embodiment, a method is provided for treating cancer comprising administering to a mammal in need thereof a combination of a compound of Formula (I) and CAR-T therapy; administering a glucocorticoid; and optionally, one or more additional anti-cancer agents. An example of a suitable glucocorticoid is dexamethasone.

In one embodiment, a method is provided for treating cancer comprising administering to a mammal in need thereof a combination of a compound of Formula (I) and CAR-T therapy; administering carboplatin; and optionally, one or more additional anti-cancer agents.

The compounds of the present invention can be formulated or co-administered with other therapeutic agents that are selected for their particular usefulness in addressing side effects associated with the aforementioned conditions. For example, compounds of the invention may be formulated with agents to prevent nausea, hypersensitivity and gastric irritation, such as antiemetics, and H₁ and H₂ antihistaminics.

In one embodiment, pharmaceutical compositions are provided comprising a compound of Formula (I) or prodrug thereof; one or more additional agents selected from a kinase inhibitory agent (small molecule, polypeptide, and antibody), an immunosuppressant, an anti-cancer agent, an anti-viral agent, anti-inflammatory agent, antifungal agent, antibiotic, or an anti-vascular hyperproliferation compound; and any pharmaceutically acceptable carrier, adjuvant or vehicle.

The above other therapeutic agents, when employed in combination with the compounds of the present invention, may be used, for example, in those amounts indicated in the Physicians' Desk Reference (PDR) or as otherwise determined by one of ordinary skill in the art. In the methods of the present invention, such other therapeutic agent(s) may be administered prior to, simultaneously with, or following the administration of the inventive compounds.

### Pharmaceutical Compositions

### Formulations (Compound I)

Also embraced within this invention is a class of pharmaceutical compositions comprising the compound of Formula (I) and one or more non-toxic, pharmaceutically-acceptable carriers and/or diluents and/or adjuvants (collectively referred to herein as “carrier” materials) and, if desired, other active ingredients.

The compounds of Formula (I) may be administered by any suitable route, preferably in the form of a pharmaceutical composition adapted to such a route, and in a dose effective for the treatment intended. The compounds and compositions of the present invention may, for example, be administered in dosage unit formulations containing conventional pharmaceutically acceptable carriers, adjuvants, and vehicles. For example, the pharmaceutical carrier may contain a mixture of mannitol or lactose and microcrystalline cellulose. The mixture may contain additional components such as a lubricating agent, e.g., magnesium stearate and a disintegrating agent such as crospovidone. The carrier mixture may be filled into a gelatin capsule or compressed as a tablet. The pharmaceutical composition may be administered as an oral dosage form or an infusion, for example.

For oral administration, the pharmaceutical composition may be in the form of, for example, a tablet, capsule, liquid capsule, suspension, or liquid. The pharmaceutical composition is preferably made in the form of a dosage unit containing a particular amount of the active ingredient. For example, the pharmaceutical composition may be provided as a tablet or capsule comprising an amount of active ingredient in the range of from about 1 to 2000 mg, preferably from about 1 to 500 mg, and more preferably from about 5 to 150 mg. A suitable daily dose for a human or other mammal may vary widely depending on the condition of the patient and other factors, but, can be determined using routine methods.

Any pharmaceutical composition contemplated herein can, for example, be delivered orally via any acceptable and suitable oral preparations. Exemplary oral preparations include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the invention can contain at least one agent selected from sweetening agents, flavoring agents, coloring agents, demulcents, antioxidants, and preserving agents.

A tablet can, for example, be prepared by admixing at least one compound of Formula (I) with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium croscarmellose, corn starch, and alginic acid; binding agents, such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasant tasting drug, or delay disintegration and absorption of the active ingredient in the gastrointestinal tract thereby sustaining the effects of the active ingredient for a longer period. Exemplary water-soluble taste masking materials, include, but are not limited to, hydroxypropyl-methylcellulose and hydroxypropyl-cellulose. Exemplary time delay materials include, but are not limited to, ethyl cellulose and cellulose acetate butyrate.

Hard gelatin capsules can, for example, be prepared by mixing at least one compound of Formula (I) with at least one inert solid diluent, such as, for example, calcium carbonate; calcium phosphate; and kaolin.

Soft gelatin capsules can, for example, be prepared by mixing at least one compound of Formula (I) with at least one water-soluble carrier, such as, for example, polyethylene glycol; and at least one oil medium, such as, for example, peanut oil, liquid paraffin, and olive oil.

An aqueous suspension can be prepared, for example, by admixing at least one compound of Formula (I) with at least one excipient suitable for the manufacture of an aqueous suspension. Exemplary excipients suitable for the manufacture of an aqueous suspension, include, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example heptadecaethylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of Formula (I) in either a vegetable oil, such as, for example, arachis oil; olive oil; sesame oil; and coconut oil; or in mineral oil, such as, for example, liquid paraffin. An oily suspension can also contain at least one thickening agent, such as, for example, beeswax; hard paraffin; and cetyl alcohol. In order to provide a palatable oily suspension, at least one of the sweetening agents already described hereinabove, and/or at least one flavoring agent can be added to the oily suspension. An oily suspension can further contain at least one preservative, including, but not limited to, for example, an antioxidant, such as, for example, butylated hydroxyanisol, and alpha-tocopherol.

Dispersible powders and granules can, for example, be prepared by admixing at least one compound of Formula (I) with at least one dispersing and/or wetting agent; at least one suspending agent; and/or at least one preservative. Suitable dispersing agents, wetting agents, and suspending agents are as already described above. Exemplary preservatives include, but are not limited to, for example, anti-oxidants, e.g., ascorbic acid. In addition, dispersible powders and granules can also contain at least one excipient, including, but not limited to, for example, sweetening agents; flavoring agents; and coloring agents.

An emulsion of at least one compound of Formula (I) can, for example, be prepared as an oil-in-water emulsion. The oily phase of the emulsions comprising compounds of Formula (I) may be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase may comprise merely an emulsifier, it may comprise a mixture of at least one emulsifier with a fat or an oil or with both a fat and an oil. Suitable emulsifying agents include, but are not limited to, for example, naturally-occurring phosphatides, e.g., soy bean lecithin; esters or partial esters derived from fatty acids and hexitol anhydrides, such as, for example, sorbitan monooleate; and condensation products of partial esters with ethylene oxide, such as, for example, polyoxyethylene sorbitan monooleate. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present invention include Tween 60, Span 80, cetostearyl alcohol, myristyl alcohol, glyceryl monostearate, sodium lauryl sulfate, glyceryl distearate alone or with a wax, or other materials well known in the art.

In another embodiment, the compounds of Formula (I) can be formulated as a nanoparticle, lipid nanoparticle, microparticle or liposome.

The compounds of Formula (I) can, for example, also be delivered intravenously, subcutaneously, and/or intramuscularly via any pharmaceutically acceptable and suitable injectable form. Exemplary injectable forms include, but are not limited to, for example, sterile aqueous solutions comprising acceptable vehicles and solvents, such as, for example, water, Ringer's solution, and isotonic sodium chloride solution; sterile oil-in-water microemulsions; and aqueous or oleaginous suspensions.

For example, the composition may be provided for intravenous administration comprising an amount of active ingredient in the range of from about 0.2 to 150 mg. In another embodiment, the active ingredient is present in the range of from about 0.3 to 10 mg. In another embodiment, the active ingredient is present in the range of from about 4 to 8.4 mg. In one embodiment, the active ingredient is administered at a dose of about 4 mg. In another embodiment, the active ingredient is administered at a dose of about 6 mg. In another embodiment, the active ingredient is administered at a dose of about 8.4 mg.

In another embodiment, the active ingredient is administered at a dose of about 0.3 mg. In another embodiment, the active ingredient is administered at a dose of about 0.6 mg. In another embodiment, the active ingredient is administered at a dose of about 1.2 mg. In another embodiment, the active ingredient is administered at a dose of about 2.4 mg.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (i.e., CAPTISOL®), cosolvent solubilization (i.e., propylene glycol) or micellar solubilization (i.e., Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid find use in the preparation of injectables.

A sterile injectable oil-in-water microemulsion can, for example, be prepared by 1) dissolving at least one compound of Formula (I) in an oily phase, such as, for example, a mixture of soybean oil and lecithin; 2) combining the Formula (I) containing oil phase with a water and glycerol mixture; and 3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butane diol; and a sterile oleaginous suspension can be prepared with a sterile non-toxic acceptable solvent or suspending medium, such as, for example, sterile fixed oils, e.g., synthetic mono- or diglycerides; and fatty acids, such as, for example, oleic acid.

Pharmaceutically acceptable carriers, adjuvants, and vehicles that may be used in the pharmaceutical compositions of this invention include, but are not limited to, ion exchangers, alumina, aluminum stearate, lecithin, self-emulsifying drug delivery systems (SEDDS) such as d-alpha-tocopherol polyethyleneglycol 1000 succinate, surfactants used in pharmaceutical dosage forms such as Tweens, polyethoxylated castor oil such as CREMOPHOR® surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutically active compounds of this invention can be processed in accordance with conventional methods of pharmacy to produce medicinal agents for administration to patients, including humans and other mammals. The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

The amounts of compounds that are administered and the dosage regimen for treating a disease condition with the compounds and/or compositions of this invention depends on a variety of factors, including the age, weight, gender, the medical condition of the subject, the type of disease, the severity of the disease, the route and frequency of administration, and the particular compound employed. Thus, the dosage regimen may vary widely, but can be determined routinely using standard methods. A daily dose of about 0.001 to 100 mg/kg body weight, preferably between about 0.005 and about 50 mg/kg body weight and most preferably between about 0.01 to 10 mg/kg body weight, may be appropriate. The daily dose can be administered in one to four doses per day.

In one embodiment, the compound is administered to the subject once a week. In another embodiment, the compound is administered to the subject once every two weeks.

For therapeutic purposes, the active compounds of this invention are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

Pharmaceutical compositions of this invention comprise at least one compound of Formula (I) and/or at least one salt thereof, and optionally an additional agent selected from any pharmaceutically acceptable carrier, adjuvant, and vehicle. Alternate compositions of this invention comprise a compound of the Formula (I) described herein, or a prodrug thereof, and a pharmaceutically acceptable carrier, adjuvant, or vehicle.

The compound in accordance with Formula (I) can be administered by any means suitable for the condition to be treated, which can depend on the need for site-specific treatment or quantity of Formula (I) compound to be delivered. The compounds and compositions of the present invention may, for example, be administered orally, mucosally, or parentally including intravascularly, intraperitoneally, subcutaneously, intramuscularly, and intrasternally. In another embodiment, the compounds and compositions of the present invention are administered intravenously.

### Formulations (CAR-T)

Compositions of the presently disclosed subject matter comprise pharmaceutical compositions comprising immunoresponsive cells expressing a BCMA-targeted or other targeted CAR and a pharmaceutically acceptable carrier. Administration can be autologous or non-autologous. For example, immunoresponsive cells expressing a CAR and compositions comprising thereof can be obtained from one subject, and administered to the same subject or a different, compatible subject. Peripheral blood derived T cells of the presently disclosed subject matter or their progeny (e.g., in vivo, ex vivo or in vitro derived) can be administered via localized injection, including catheter administration, systemic injection, localized injection, intravenous injection, or parenteral administration. When administering a pharmaceutical composition of the presently disclosed subject matter (e.g., a pharmaceutical composition comprising immunoresponsive cells expressing a BCMA-targeted CAR), it can be formulated in a unit dosage injectable form (solution, suspension, emulsion).

Immunoresponsive cells expressing a CAR and compositions comprising thereof of the presently disclosed subject matter can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions comprising immunoresponsive cells expressing a generally BCMA-targeted CAR of the presently disclosed subject matter in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, alum inurn monostearate and gelatin. According to the present invention, however, any vehicle, diluent, or additive used would have to be compatible with the immunoresponsive cells expressing a generally targeted CAR of the presently disclosed subject matter.

The compositions can be isotonic, i.e., they can have the same osmotic pressure as blood and lacrimal fluid. The desired isotonicity of the compositions of the presently disclosed subject matter may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the immunoresponsive cells as describe in the presently disclosed subject matter. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of the immunoresponsive cells of the presently disclosed subject matter is the quantity of cells necessary to achieve an optimal effect. The quantity of cells to be administered will vary for the subject being treated. In certain embodiments, from about 10⁴ to about 10¹⁰, from about 10⁵ to about 10⁹, or from about 10⁶ to about 10⁸ immunoresponsive cells of the presently disclosed subject matter are administered to a subject. More effective cells may be administered in even smaller numbers. In certain embodiments, at least about 1×10⁸, about 2×10⁸, about 3×10⁸, about 4×10⁸, and about 5×10⁸ immunoresponsive cells of the presently disclosed subject matter are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods of the presently disclosed subject matter. Typically, any additives (in addition to the active cell(s) and/or agent(s)) are present in an amount of from about 0.001% to about 50% by weight) solution in phosphate buffered saline, and the active ingredient is present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

For adoptive immunotherapy using antigen-specific T cells, cell doses in the range of about 10⁶ to about 10¹⁰ (e.g., about 10⁹) are typically infused. Upon administration of the immunoresponsive cells into the subject and subsequent differentiation, the immunoresponsive cells are induced that are specifically directed against one specific antigen (e.g., BCMA). “Induction” of T cells can include inactivation of antigen-specific T cells such as by deletion or anergy. Inactivation is particularly useful to establish or reestablish tolerance such as in autoimmune disorders. The immunoresponsive cells of the presently disclosed subject matter can be administered by any methods known in the art, including, but not limited to, pleural administration, intravenous administration, subcutaneous administration, intranodal administration, intratumoral administration, intrathecal administration, intrapleural administration, intraperitoneal administration, and direct administration to the thymus. In certain embodiments, the immunoresponsive cells and the compositions comprising thereof are intravenously administered to the subject in need.

### Methods of Use

In one embodiment, the present invention provides the use of the described compounds or compositions for treating, suppressing or inhibiting a B-cell maturation antigen (BCMA)-related disorder or a disease associated with a BCMA expression in a subject. In one embodiment, the BCMA-related disorder or a disease associated with a BCMA expression comprises a lymphoma.

In one embodiment, the present invention provides a method of improving the efficacy of a BCMA-targeting immuno-therapeutic in a subject having lymphoma comprising the step of administering to said subject a first composition comprising one or more compounds represented by the structure of Formula (I):

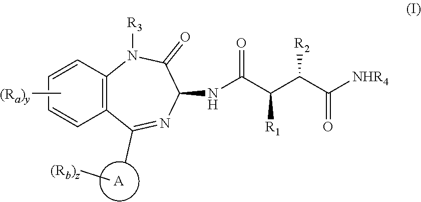

and/or at least one salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

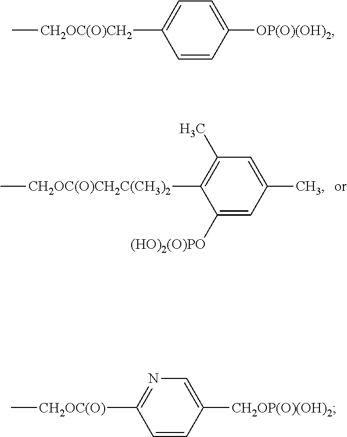

R_{y} is: —SCH₂CH(NH₂)C(O)OH, —SCH₂CH(NH₂)C(O)OCH₃, or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is 1 or 2,
   and a second composition comprising one or more BCMA-targeting immuno-therapeutics.

In another embodiment, the present invention provides a method of treating, suppressing or inhibiting a lymphoma in a subject comprising the step of administering to said subject a first composition comprising one or more compounds represented by the structure of Formula (I):

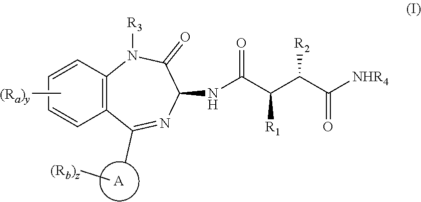

and/or at least one salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

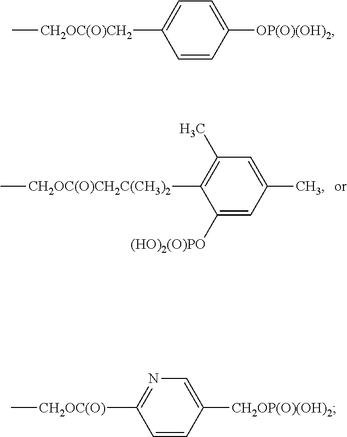

R_{y} is: —SCH₂CH(NH₂)C(O)OH, —SCH₂CH(NH₂)C(O)OCH₃, or —SCH₂CH(NH₂)C(O)OC(CH₃)₃;
Ring A is phenyl or pyridinyl;
each Rₐ is independently F, Cl, —CN, —OCH₃, CI-3 alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is 1 or 2,
   and a second composition comprising one or more BCMA-targeting immuno-therapeutics.

In another embodiment, the present invention provides a method of decreasing B-cell maturation antigen (BCMA) shedding or decreasing soluble BCMA in a lymphoma cell in a subject having lymphoma comprising the step of administering to said subject a composition comprising one or more compounds represented by the structure of Formula (I):

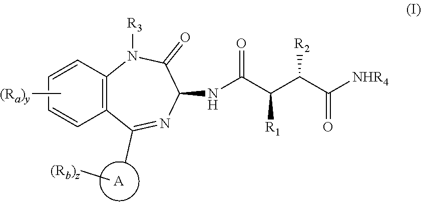

and/or at least one salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

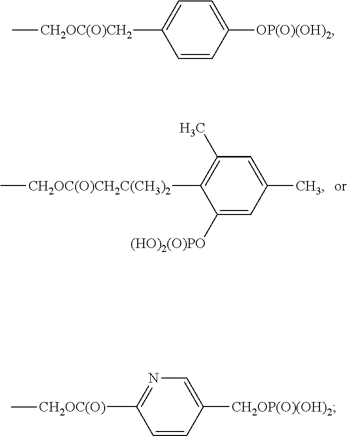

R_{y} is: —SCH₂CH(NH₂)C(O)OH, —SCH₂CH(NH₂)C(O)OCH₃, or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is 1 or 2.

In one embodiment, the lymphoma comprises diffuse large B-cell lymphoma (DLBCL). In another embodiment, the lymphoma comprises Burkitt's Lymphoma. In another embodiment, the lymphoma comprises a B-cell Lymphoma.

In another embodiment, the lymphoma comprises a Hodgkin's Lymphoma. In another embodiment, the lymphoma comprises a Mantle cell lymphoma (MCL). In another embodiment, the lymphoma comprises a non-Hodgkin's Lymphoma (NHL).

In another embodiment, the lymphoma does not comprise a B-cell Acute Lymphoblastic Leukemia/Lymphoma (B-ALL). In another embodiment, the lymphoma does not comprise a Histiocytic Lymphoma. In another embodiment, the lymphoma does not comprise an Anaplastic Large Cell Lymphoma. In another embodiment, the lymphoma does not comprise a Cutaneous T-cell Lymphoma.

In one embodiment, the lymphoma comprises an extranodal lymphoma. In another embodiment, the lymphoma comprises a marginal zone B cell lymphoma. In another embodiment, the lymphoma comprises a B-ALL. In another embodiment, the lymphoma comprises a Histiocytic Lymphoma. In another embodiment, the lymphoma comprises an Anaplastic Large Cell Lymphoma. In another embodiment, the lymphoma comprises a Cutaneous T-cell Lymphoma.

In one embodiment, the lymphoma has low levels of BCMA. In another embodiment, the lymphoma has undetectable levels of BCMA. In one embodiment, the BCMA levels are under the threshold for allowing anti-BCMA therapies to be effective.

In one embodiment, the BCMA is expressed on the cell surface. In another embodiment, the BCMA comprises soluble BCMA.

In another embodiment, the present invention also provides a method for treating, suppressing or inhibiting a B-cell maturation antigen (BCMA)-related disorder or a disease associated with a BCMA expression in a subject comprising the step of administering to said subject a first composition comprising one or more B-cell maturation antigen (BCMA)-targeting immuno-therapeutics and a second composition comprising one or more compounds represented by the structure of Formula (I):

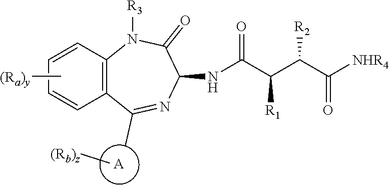

and/or at least one salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

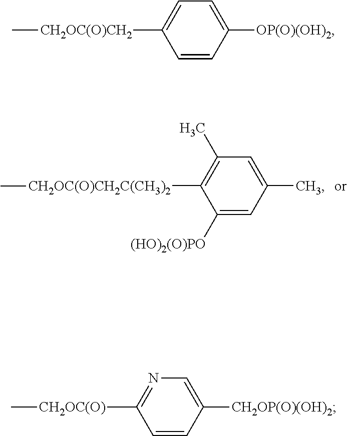

R_{y} is: —SCH₂CH(NH₂)C(O)OH, —SCH₂CH(NH₂)C(O)OCH₃, or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is zero, 1, or 2.

In another embodiment, z is 1 or 2.

In one embodiment, the BCMA-related disorder comprises a proliferative disease. In another embodiment, the BCMA-related disorder comprises multiple myeloma, acute myelomonocytic leukemia (AMML) with eosinophilia, T cell lymphoma, acute monocytic leukemia, follicular lymphoma, diffuse large B-cell lymphoma (DLBCL), mantle cell lymphoma (MCL), or a combination thereof.

In another embodiment, the present invention provides the use of the described compounds or compositions for treating, suppressing or inhibiting a proliferative disease in a subject. In another embodiment, the present invention provides a method of treating, suppressing or inhibiting a proliferative disease in a subject. comprising the step of administering to said subject a first composition comprising one or more chimeric antigen receptor T cells (CAR-T cells) that express a tumor antigen cleaved by gamma secretase and a second composition comprising one or more gamma secretase inhibitors comprising a compound of Formula (I):

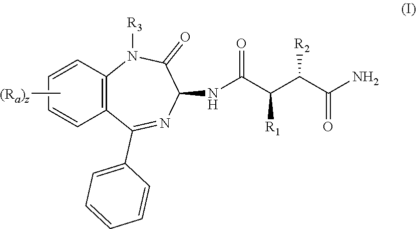

wherein:
R₁ is CH₂CF₃ or CH₂CH₂CF₃;
R₂ is CH₂CF₃, CH₂CH₂CF₃, or CH₂CH₂CH₂CF₃;
R₃ is H or CH₃;
each Rₐ is independently F, Cl, CN, OCH₃, and/or NHCH₂CH₂OCH₃; and
z is zero, 1, or 2.

In another embodiment, the present invention also provides a method of treating, suppressing or inhibiting a proliferative disease in a subject comprising the step of administering to said subject a first composition comprising one or more B-cell maturation antigen (BCMA)-targeting immuno-therapeutics and a second composition comprising one or more compounds represented by the structure of Formula (I):

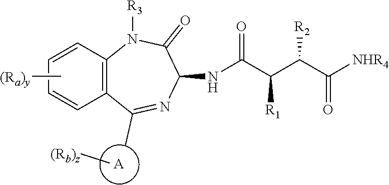

and/or at least one salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

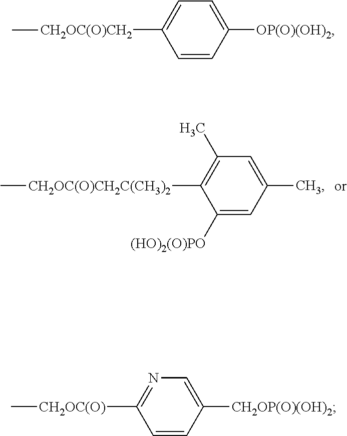

R_{y} is: —SCH₂CH(NH₂)C(O)OH, —SCH₂CH(NH₂)C(O)OCH₃, or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is zero, 1, or 2.

In another embodiment, z is 1 or 2.

In one embodiment, the present invention provides the use of the described compounds or compositions for improving the efficacy of an anti-B-cell maturation antigen (BCMA) therapy in a subject.

In another embodiment, the present invention also provides a method of improving the efficacy of an anti-B-cell maturation antigen (BCMA) therapy in a subject comprising the step of administering to said subject a first composition comprising one or more B-cell maturation antigen (BCMA)-targeting immuno-therapeutics and a second composition comprising one or more compounds represented by the structure of Formula (I):

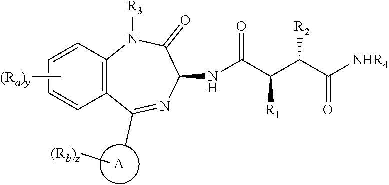

and/or at least one salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC (O)CH(NH₂)CH(CH₃)₂,

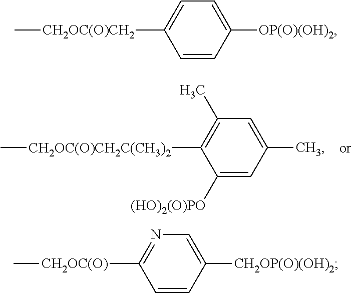

R_{y} is: —SCH₂CH(NH₂)C(O)OH, —SCH₂CH(NH₂)C(O)OCH₃, or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is zero, 1, or 2.

In another embodiment, z is 1 or 2.

In one embodiment, the anti-B-cell maturation antigen (BCMA) therapy comprises an anti-cancer therapy.

In one embodiment, the present invention provides the use of the described compound or compositions for decreasing B-cell maturation antigen (BCMA) shedding from a cancer cell in a subject.

In another embodiment, the present invention also provides a method of decreasing B-cell maturation antigen (BCMA) shedding from a cancer cell in a subject comprising the step of administering to said subject a composition comprising one or more compounds represented by the structure of Formula (I):

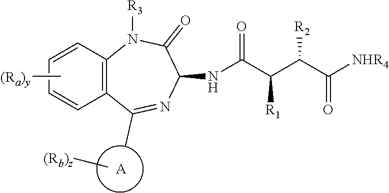

and/or at least one salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

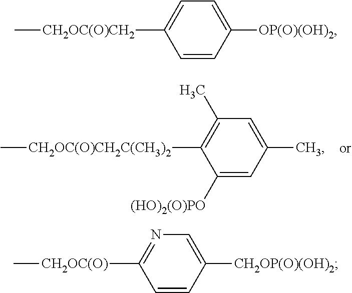

R_{y} is: —SCH₂CH(NH₂)C(O)OH, —SCH₂CH(NH₂)C(O)OCH₃, or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is zero, 1, or 2.

In another embodiment, z is 1 or 2.

In another embodiment, the present invention also provides a method of decreasing B-cell maturation antigen (BCMA) shedding from a cell in a subject comprising the step of administering to said subject a composition comprising one or more compounds represented by the structure of Formula (I) as described herein. In one embodiment, the cell comprises a late memory B cell committed to plasma cell (PC) differentiation or a PC. In another embodiment, the cell comprises a multiple myeloma cell.

In one embodiment, the present invention provides the use of a therapeutically acceptable amount of one or more compounds or compositions as described herein for treating, suppressing or inhibiting a proliferative disease in a subject. In another embodiment, the present invention provides the use of a therapeutically effective amount of one or more compounds or compositions as described herein for treating, suppressing or inhibiting a proliferative disease in a subject. In another embodiment, the present invention provides the use of a synergistically effective amount of one or more compounds or compositions as described herein for treating, suppressing or inhibiting a proliferative disease in a subject. In another embodiment, the present invention provides the use of a synergistically therapeutically effective amount of one or more compounds or compositions as described herein for treating, suppressing or inhibiting a proliferative disease in a subject.

In one embodiment, the proliferative disease comprises a Desmoid tumor.

In one embodiment, the proliferative disease comprises a pre-cancerous condition or a benign proliferative disorder.

In one embodiment, the term “pre-cancerous” or, alternatively, “pre-malignant” as used herein interchangeably refers to diseases, syndromes or other conditions associated with an increased risk of cancer. Pre-cancer conditions in the context of the present invention include, but are not limited to: breast calcifications, vaginal intra-epithelial neoplasia, Barrett's esophagus, atrophic gastritis, dyskeratosis congenital, sideropenic dysphagia, lichen planus, oral submucous fibrosis, actinic keratosis, solar elastosis, cervical dysplasia, leukoplakia and erythroplakia.

In one embodiment, the term “benign hyperproliferative disorder” as used herein refers to a condition in which there is an abnormal growth and differentiation of cells and an increase in the amount of organic tissue that results from cell proliferation. The benign hyperproliferative disorder may be attributed to lack of response or inappropriate response to regulating factors, or alternatively to dysfunctional regulating factors. Non-limiting examples of benign hyperproliferative disorder are psoriasis and benign prostatic hyperplasia (BPH).

In another embodiment, the proliferative disease comprises a cancer.

In one embodiment, the cancer comprises a solid tumor. In another embodiment, the cancer comprises a hematological malignancy.

In one embodiment, a subject as described herein has cancer. In one embodiment, the term “cancer” in the context of the present invention includes all types of neoplasm whether in the form of solid or non-solid tumors, and includes both malignant and premalignant conditions as well as their metastasis.

In one embodiment, the cancer is a carcinoma, sarcoma, myeloma, leukemia, or lymphoma. In another embodiment, the cancer is a mixed type.

In one embodiment, Mixed Type cancers contain several types of cells. The type components may be within one category or from different categories. Some examples are: adenosquamous carcinoma; mixed mesodermal tumor; carcinosarcoma; teratocarcinoma.

In another embodiment, the cancer is dependent upon Notch activation. In another embodiment, the cancer is not dependent upon Notch activation.

In another embodiment, the carcinoma comprises Adenoid Cystic Carcinoma (ACC). In another embodiment, the carcinoma comprises Gastro-esophageal junction carcinoma.

In one embodiment, the carcinoma is an adenocarcinoma. In another embodiment, the carcinoma is a squamous cell carcinoma.

In one embodiment, the sarcoma comprises osteosarcoma or osteogenic sarcoma (bone); Chondrosarcoma (cartilage); Leiomyosarcoma (smooth muscle); Rhabdomyosarcoma (skeletal muscle); Mesothelial sarcoma or mesothelioma (membranous lining of body cavities); Fibrosarcoma (fibrous tissue); Angiosarcoma or hemangioendothelioma (blood vessels); Liposarcoma (adipose tissue); Glioma or astrocytoma (neurogenic connective tissue found in the brain); Myxosarcoma (primitive embryonic connective tissue); and Mesenchymous or mixed mesodermal tumor (mixed connective tissue types).

In one embodiment, the cancer comprises myeloma, which, in one embodiment, is cancer that originates in the plasma cells of bone marrow. The plasma cells produce some of the proteins found in blood. In one embodiment, the cancer comprises multiple myeloma.

In another embodiment, the cancer comprises leukemia (“non-solid tumor” or “blood cancer”), which in one embodiment, is a cancer of the bone marrow (the site of blood cell production). In one embodiment, leukemia comprises myelogenous or granulocytic leukemia (malignancy of the myeloid and granulocytic white blood cell series); Lymphatic, lymphocytic, or lymphoblastic leukemia (malignancy of the lymphoid and lymphocytic blood cell series); and Polycythemia vera or erythremia (malignancy of various blood cell products, but with red cells predominating).

In another embodiment, the cancer comprises T-cell acute lymphoblastic leukemia (T-ALL). In another embodiment, the cancer comprises T-lymphoblastic leukemia/lymphoma (TLL). In another embodiment, the cancer comprises Chronic Lymphocytic Leukemia (CLL).

In another embodiment, the cancer comprises a lymphoma.

In another embodiment, the cancer is dependent upon Notch activation. In another embodiment, the cancer comprises a Notch-activating mutation. In another embodiment, the cancer is not dependent upon Notch activation.

In one embodiment, a cancer as described herein comprises a Notch activating genetic alteration. In another embodiment, a cancer as described herein comprises a Notch activating alteration. In another embodiment, a cancer as described herein comprises a Notch activating mutation. In another embodiment, a cancer as described herein comprises a Notch activating genetic mutation. In another embodiment, a cancer as described herein comprises a Notch mutation. In another embodiment, a cancer as described herein comprises a Notch altering mutation.

In one embodiment, Notch activating genetic alterations comprise mutation in one or more Notch related genes.

In one embodiment, the mutation in one or more Notch-related genes induces a gain of function (GOF) in Notch activity.

In another embodiment, the mutation in one or more Notch-related genes comprises a missense mutation. In another embodiment, the mutation in one or more Notch-related genes comprises a nonsense mutation. In another embodiment, the mutation in one or more Notch-related genes comprises an insertion mutation. In another embodiment, the mutation in one or more Notch-related genes comprises a deletion mutation. In another embodiment, the mutation in one or more Notch-related genes comprises a duplication mutation. In another embodiment, the mutation in one or more Notch-related genes comprises a frameshift mutation. In another embodiment, the mutation in one or more Notch-related genes comprises a repeat expansion. In another embodiment, the mutation in one or more Notch-related genes comprises a fusion.

In one embodiment, the B-cell maturation antigen (BCMA)-related disorder or a disease associated with BCMA expression comprises one or more cells with an activating Notch signature. In one embodiment, the activating Notch signature comprises upregulation of expression of one or more Notch-related genes. In another embodiment, the activating Notch signature comprises downregulation of expression of one or more Notch-related genes. In another embodiment, the activating Notch signature comprises upregulation of expression of some Notch-related genes and downregulation of expression of some Notch-related genes.

In another embodiment, the cancer comprises astrocytoma, bladder cancer, breast cancer, cholangiocarcinoma (CCA), colon cancer, colorectal cancer, colorectal carcinoma, epithelial carcinoma, epithelial ovarian cancers, fibrosarcoma, gall bladder cancer, gastric cancer, glioblastoma, glioma, head and neck cancer, hepatocellular carcinoma, kidney cancer, liver cancer, lung cancer including non-small cell lung cancer (NSCLC), malignant fibrous histiocytoma (MFH), malignant pleural mesothelioma (MPM), medulloblastoma, melanoma, mesothelioma, neuroblastoma, osteosarcoma, ovarian adenocarcinoma, ovarian cancer, pancreatic adenocarcinoma, pancreatic cancer, prostate cancer, renal cell carcinoma (RCC), rhabdomyosarcoma, seminal vesicle cancer, and thyroid cancer.

In one embodiment, the breast cancer is triple negative breast cancer.

As used herein, the term “cancer” includes the above categories of carcinoma, sarcoma, myeloma, leukemia, lymphoma and mixed type tumors. In particular, the term cancer includes: lymphoproliferative disorders, breast cancer, ovarian cancer, prostate cancer, cervical cancer, endometrial cancer, lung cancer, bone cancer, liver cancer, stomach cancer, bladder cancer, colon cancer, colorectal cancer, pancreatic cancer, cancer of the thyroid, head and neck cancer, cancer of the central nervous system, brain cancer, cancer of the peripheral nervous system, skin cancer, kidney cancer, as well as metastases of all the above. More particularly, as used herein the term may refer to: hepatocellular carcinoma, hematoma, hepatoblastoma, rhabdomyosarcoma, esophageal carcinoma, thyroid carcinoma, ganglioblastoma, glioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, squamous cell carcinoma, basal cell carcinoma, adenocarcinoma (well differentiated, moderately differentiated, poorly differentiated or undifferentiated), renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma, Waldenstrom's Macroglobulinemia.

In another embodiment, the administration of any of the compositions as described herein reduces growth of the cells of a solid tumor or hematological malignancy by 40%, 50%, 60%, 70%, 80%, 90% or 95% compared to growth of the cells of the solid tumor or hematological malignancy that have not been treated with the compositions. In the case of combination treatments, the administration of any of the described combinations reduces growth of the cells of a solid tumor or hematological malignancy compared to subjects treated with either one of the compositions, via a different cancer treatment, or who have not been treated. In another embodiment, the present invention provides methods of increasing or lengthening survival of a subject having a neoplasia. As used herein, the term “neoplasia” refers to a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasias can affect a variety of cell types, tissues, or organs, including but not limited to an organ selected from the group consisting of bladder, colon, bone, brain, breast, cartilage, glia, esophagus, fallopian tube, gallbladder, heart, intestines, kidney, liver, lung, lymph node, nervous tissue, ovaries, pleura, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as sarcomas, carcinomas, or plasmacytomas (malignant tumor of the plasma cells).

In another embodiment, the present invention provides a method of treating a subject with a B cell condition or disorder or preventing BCMA shedding from a B cell in a subject comprising administering to the subject a combination of a gamma secretase inhibitor as described herein and CAR-T cells as described herein. In another embodiment, the present invention provides a method of treating pathogenic B cells.

In one embodiment, a subject as described herein is being treated with or has been previously treated with radiation therapy, chemotherapy, transplantation, immunotherapy, hormone therapy, or photodynamic therapy.

### Definitions

Unless specifically stated otherwise herein, references made in the singular may also include the plural. For example, “a” and “an” may refer to either one, or one or more.

The definitions set forth herein take precedence over definitions set forth in any patent, patent application, and/or patent application publication incorporated herein by reference.

Listed below are definitions of various terms used to describe the present invention. These definitions apply to the terms as they are used throughout the specification (unless they are otherwise limited in specific instances) either individually or as part of a larger group.

As used herein, the term “administering” refers to bringing in contact with a compound of the present invention. In one embodiment, the compositions are applied locally. In another embodiment, the compositions are applied systemically. Administration can be accomplished to cells or tissue cultures, or to living organisms, for example humans.

As used herein, the terms “administering,” “administer,” or “administration” refer to deliver one or more compounds or compositions to a subject parenterally, enterally, or topically. Illustrative examples of parenteral administration include, but are not limited to, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. Illustrative examples of enteral administration include, but are not limited to oral, inhalation, intranasal, sublingual, and rectal administration. Illustrative examples of topical administration include, but are not limited to, transdermal and vaginal administration. In particular embodiments, an agent or composition is administered parenterally, optionally by intravenous administration or oral administration to a subject.

In one embodiment, a composition of the present invention comprises a pharmaceutically acceptable composition. In one embodiment, the phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In one embodiment, a composition of the present invention is administered in a therapeutically effective amount. In one embodiment, a “therapeutically effective amount” is intended to include an amount of a compound of the present invention alone or an amount of the combination of compounds claimed or an amount of a compound of the present invention in combination with other active ingredients effective to act as an inhibitor to a NOTCH receptor, effective to inhibit gamma secretase, or effective to treat or prevent proliferative diseases such as cancer. In one embodiment, a “therapeutically effective amount” of a composition of the invention is that amount of composition which is sufficient to provide a beneficial effect to the subject to which the composition is administered.

As used herein, “treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting its development; and/or (c) relieving the disease-state, i.e., causing regression of the disease state.

In one embodiment, “treating” refers to both therapeutic treatment and prophylactic or preventative measures, wherein the object is to prevent or lessen the targeted pathologic condition or disorder as described hereinabove. Thus, in one embodiment, treating may include directly affecting or curing, suppressing, inhibiting, preventing, reducing the severity of, delaying the onset of, reducing symptoms associated with the disease, disorder or condition, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In one embodiment, the term “decreasing the size of the tumor” as used herein is assessed using the “Response Evaluation Criteria In Solid Tumors” (RECIST). In one embodiment, RECIST measures reduction in tumor size by measuring the longest dimension of a target lesion. In one embodiment, the target lesion is selected on the basis of its size (lesion with the longest diameter) and its suitability for accurate repeated measurements (either by imaging techniques or clinically). In one embodiment, all other lesions (or sites of disease) are identified as non-target lesions and are also recorded at baseline. Measurements of these lesions are not required, but the presence or absence of each is noted throughout follow-up.

In one embodiment, the term “decreasing the volume of the tumor” as used herein is assessed using the radiological tumor response evaluation criteria. Whereby, the tumor is measured in two dimensions its maximum diameter (width) in the translation plane and its largest perpendicular diameter on same image (thickness), according to the World Health Organization (WHO).

According to any of the methods of the present invention and in one embodiment, a subject as described herein is human. In another embodiment, the subject is mammalian. In another embodiment, the subject is a primate, which in one embodiment, is a non-human primate. In another embodiment, the subject is murine, which in one embodiment is a mouse, and, in another embodiment is a rat. In another embodiment, the subject is canine, feline, bovine, equine, caprine, ovine, porcine, simian, ursine, vulpine, or lupine. In one embodiment, the subject is a chicken or fish.

In one embodiment, the compositions as described herein comprise the components of the composition (i.e., one or more chimeric antigen receptor T cells (CAR-T cells) that express a tumor antigen cleaved by gamma secretase and one or more gamma secretase inhibitors comprising a compound of Formula (I)) as described herein. In another embodiment, the compositions as described herein consist of the components of the composition (i.e., one or more chimeric antigen receptor T cells (CAR-T cells) that express a tumor antigen cleaved by gamma secretase and one or more gamma secretase inhibitors comprising a compound of Formula (I)) as described herein. In another embodiment, the compositions as described herein consist essentially of the components of the composition (i.e., one or more chimeric antigen receptor T cells (CAR-T cells) that express a tumor antigen cleaved by gamma secretase and one or more gamma secretase inhibitors comprising a compound of Formula (I)) as described herein.

It is to be understood that the compositions and methods of the present invention comprising the elements or steps as described herein may, in another embodiment, consist of those elements or steps, or in another embodiment, consist essentially of those elements or steps. In some embodiments, the term “comprise” refers to the inclusion of the indicated active agents, such as the CAR-T cells and the gamma secretase inhibitor, as well as inclusion of other active agents, and pharmaceutically or physiologically acceptable carriers, excipients, emollients, stabilizers, etc., as are known in the pharmaceutical industry. In some embodiments, the term “consisting essentially of” refers to a composition, whose only active ingredients are the indicated active ingredients. However, other compounds may be included which are for stabilizing, preserving, etc. the formulation, but are not involved directly in the therapeutic effect of the indicated active ingredients. In some embodiments, the term “consisting essentially of” may refer to components which facilitate the release of the active ingredient. In some embodiments, the term “consisting” refers to a composition, which contains the active ingredients and a pharmaceutically acceptable carrier or excipient.

### Timing and Site of Administration

In one embodiment, the administration of the CAR T-cells occurs prior to, concurrent with, or following the administration of the compound of Formula (I).

In one embodiment, the administration of the CAR-T cells occurs at the same site as the administration of the compound of Formula (I).

In one embodiment, the compound of Formula (I) is administered several days before and several days after the administration of CAR-T cells. In one embodiment, the compound of Formula (I) is administered 1, 2, 3, 4, or 5 days prior to the administration of CAR-T cells. In another embodiment, the compound of Formula (I) is administered 6, 7, 8, 9, or 10 days prior to the administration of CAR-T cells. In one embodiment, the compound of Formula (I) is administered 1, 2, 3, 4, or 5 days subsequent to the administration of CAR-T cells. In another embodiment, the compound of Formula (I) is administered 6, 7, 8, 9, or 10 days subsequent to the administration of CAR-T cells. In another embodiment, the compound of Formula (I) is administered 1, 2, 3, or 4 weeks subsequent to the administration of CAR-T cells.

In another embodiment, the compound of Formula (I) is administered one day before and up to 9 days following CAR-T cell administration. In another embodiment, the compound of Formula (I) is administered one day before and on days 1, 8, and 9 following CAR-T cell administration. In another embodiment, the compound of Formula (I) is administered one day before and 9 days following CAR-T cell administration. In another embodiment, the compound of Formula (I) is administered one day before and daily for 9 days following CAR-T cell administration. In another embodiment, the compound of Formula (I) is administered one day before and on day 9 following CAR-T cell administration.

In some embodiments, one or more compositions of the present invention are administered at least once during a treatment cycle. In some embodiments, the compositions of the present invention are administered to the subject on the same days. In some embodiments, the compositions of the present invention are administered to the subject on the different days. In some embodiments, one or more compositions of the present invention are administered to the subject on the same days and on different days according to treatment schedules.

In particular embodiments, one or more compositions of the present invention are administered to the subject over one or more treatment cycles. A treatment cycle can be at least two, at least three, at least four, at least five, at least six, at least seven, at least 14, at least 21, at least 28, at least 48, or at least 96 days or more. In one embodiment, a treatment cycle is 28 days. In certain embodiments, the compositions are administered over the same treatment cycle or concurrently over different treatment cycles assigned for each composition. In various embodiments, the treatment cycle is determined by a health care professional based on conditions and needs of the subject.

In some embodiments, a composition is administered on at least one day, at least two days, at least three days, at least four days, at least five days, at least six days, at least seven days, at least eight days, at least nine days, at least ten days, at least eleven days, at least twelve days, at least 13 days, at least 14 days, at least 21 days, or all 28 days of a 28 day treatment cycle. In particular embodiments, a composition is administered to a subject once a day. In other particular embodiments, a composition is administered twice a day. In certain embodiments a composition is administered more than twice a day.

In one embodiment, one or more of the compositions as described herein are administered once per day. In another embodiment, one or more of the compositions as described herein are administered twice per day. In another embodiment, one or more of the compositions as described herein are administered three times per day. In another embodiment, one or more of the compositions as described herein are administered four times per day. In another embodiment, one or more of the compositions as described herein are administered once every two days, once every three days, twice a week, once a week, once every 2 weeks, once every 3 weeks.

In one embodiment, one or more of the compositions as described herein are administered for 7 days to 28 days. In another embodiment, one or more of the compositions as described herein are administered for 7 days to 8 weeks. In another embodiment, one or more of the compositions as described herein are administered for 7 days to 50 days. In another embodiment, one or more of the compositions as described herein are administered for 7 days to six months. In another embodiment, one or more of the compositions as described herein are administered for 7 days to one and half years. In another embodiment, one or more of the compositions as described herein are administered for 14 days to 12 months. In another embodiment, one or more of the compositions as described herein are administered for 14 days to 3 years. In another embodiment, one or more of the compositions as described herein are administered for several years. In another embodiment, one or more of the compositions as described herein are administered for one month to six months.

In one embodiment, one or more of the compositions as described herein are administered for 7 days. In another embodiment, one or more of the compositions as described herein are administered for 14 days. In another embodiment, one or more of the compositions as described herein are administered for 21 days. In another embodiment, one or more of the compositions as described herein are administered for 28 days. In another embodiment, one or more of the compositions as described herein are administered for 50 days. In another embodiment, one or more of the compositions as described herein are administered for 56 days. In another embodiment, one or more of the compositions as described herein are administered for 84 days. In another embodiment, one or more of the compositions as described herein are administered for 90 days. In another embodiment, one or more of the compositions as described herein are administered for 120 days.

The number of times a composition is administered to a subject in need thereof depends on the discretion of a medical professional, the disorder, the severity of the disorder, and the subject's response to the formulation. In some embodiments, a composition disclosed herein is administered once to a subject in need thereof with a mild acute condition. In some embodiments, a composition disclosed herein is administered more than once to a subject in need thereof with a moderate or severe acute condition. In the case wherein the subject's condition does not improve, upon the doctor's discretion the composition may be administered chronically, that is, for an extended period of time, including throughout the duration of the subject's life in order to ameliorate or otherwise control or limit the symptoms of the subject's disease or condition.

In the case wherein the subject's status does improve, upon the doctor's discretion the composition may administered continuously; or, the dose of drug being administered may be temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday may be from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

### Kits

The present invention further comprises combinations of the compositions of the present invention and, optionally, one or more additional agents in kit form, e.g., where they are packaged together or placed in separate packages to be sold together as a kit, or where they are packaged to be formulated together.

In certain embodiments, the kit comprises a therapeutic or prophylactic composition containing an effective amount of an immunoresponsive cell comprising a BCMA- or other-targeted CAR in unit dosage form and an effective amount of the gamma secretase inhibitor compound, as described herein. In particular embodiments, the cells further expresses at least one co-stimulatory ligand. In certain embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the immunoresponsive cell is provided together with instructions for administering the cell to a subject having or at risk of developing a neoplasia (e.g., multiple myeloma). The instructions will generally include information about the use of the composition for the treatment or prevention of a neoplasia (e.g., multiple myeloma). In other embodiments, the instructions include at least one of the following: description of the therapeutic agent; dosage schedule and administration for treatment or prevention of a neoplasia (e.g., multiple myeloma) or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

### Example 1

### Effect of γ-Secretase Inhibitors on B-Cell Maturation Antigen (BCMA) Expression in Multiple Myeloma (MM) Cell Lines

The basal cell surface expression of BCMA on MM cells and the effect of γ-secretase inhibitors on a) the expression of BCMA on the cell surface as well as b) the level of soluble BCMA were examined.

For detecting levels of soluble and cell-bound BCMA, U266 cells (MM cell line) were seeded at 1×10⁵ cells/well in a total volume of 250 μl into a 96-well plate. The cells were cultured in RPMI medium supplemented with 10% FCS. GSIs (Compound 1, Compound 22, LY3039478 (Lilly), and PF3084014 (Pfizer)) were added to U266 cell cultures at a concentration of 0.3 nM, 0.5 nM, 1 nM and 3 nM for soluble BCMA and at a concentration of 0.3 nM, 1 nM, 3 nM and 10 nM for cell-bound BCMA. Following 24 hours of incubation at 37° C., the cells were collected, and BCMA levels were evaluated in both the cells (cell-bound BCMA) and the cell media (soluble BCMA). Cells were stained with an anti-BCMA antibody (PE anti-human CD269 BCMA, Biolegend). Soluble BCMA levels were analyzed by ELISA (Human BCMA/TNFRSF17 DuoSet kit: R&D Systems).

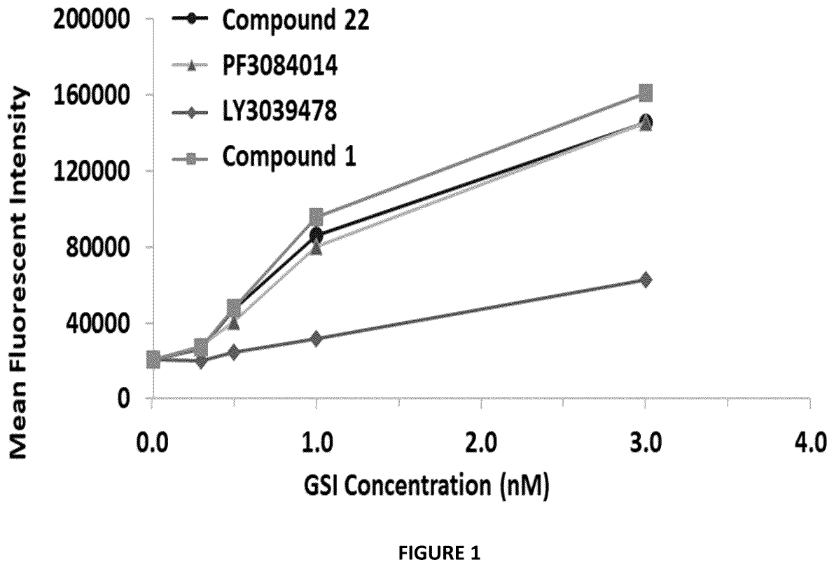

First, basal level BCMA expression was measured by fluorescence-activated cell sorting (FACS) in non-treated cells. BCMA was present on the cell surface of the MM cell line (FIG. 1. GSI concentration 0 nM).

Incubation of MM cells with Compound 1, Compound 22, LY3039478, and PF3084014 increased cell-bound BCMA levels (FIG. 1). Compounds 1 and 22 were as effective as LY3039478 and more effective than PF3084014 at increasing cell-bound BCMA at all doses above 0.3 nM.

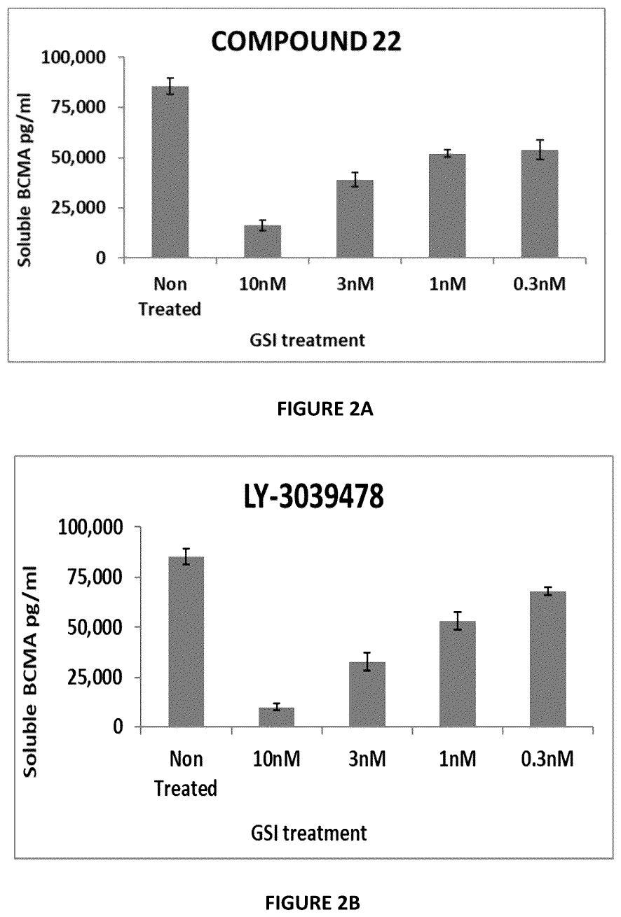

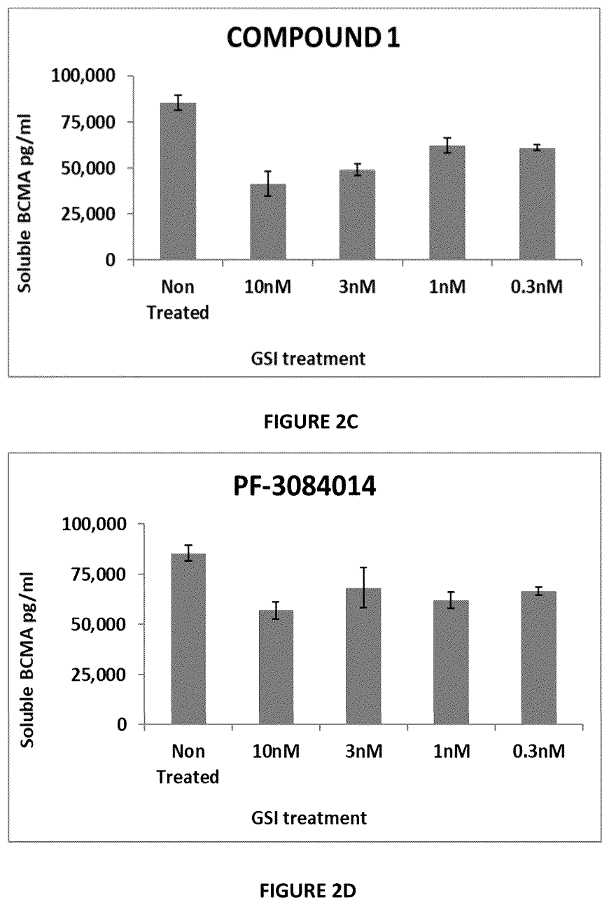

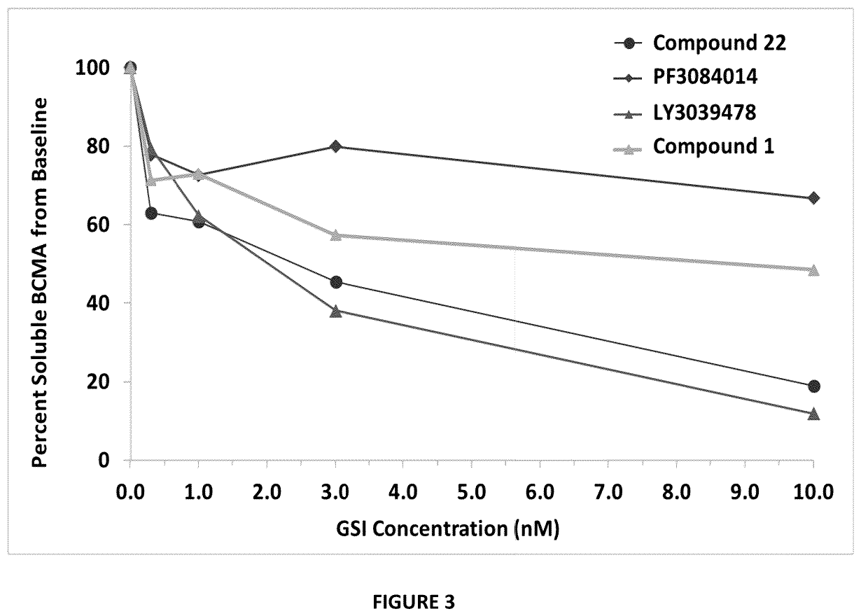

Incubation of MM cells with GSIs also reduced soluble BCMA in cell media (FIGS. 2-3). Compound 22 was more potent than Compound 1 in reducing soluble BCMA in cell media (FIGS. 2A, 2C, and 3). and was in fact as potent as LY3039478 (FIG. 2B) and more potent than PF3084014 (FIG. 2D, FIG. 3).

BCMA is a protein that is highly expressed on MM cells. BCMA is actively shed from MM cells, a process which is mediated by gamma secretase. It was demonstrated herein that Compounds 1 and 22 inhibit BCMA shedding (e.g., increase BCMA levels on the target MM cells and decrease levels of soluble BCMA) as well as or better than other GSIs. Since soluble BCMA can sequester anti-BCMA therapies, such as BCMA CAR-T and anti-BCMA bi-specific monoclonal antibodies, and hamper treatment, GSIs that inhibit BCMA shedding such as Compounds 1 and 22 are good candidates for combination with anti-BCMA therapies.

### Examples 2-4: Materials and Methods

### Construct of BCMA-Specific CAR-T Cells

Multiple unique fully human scFvs to BCMA are generated, and CARs based on these scFvs are generated. Multiple scFvs are identified by screening a fully human scFv phage library (>6×10¹⁰ scFvs) with BCMA-Fc fusion protein and then 3T3 cells expressing human BCMA. FACS analysis of phage antibody clones against BCMA-3T3 and parental 3T3 cell lines is used to confirm unique positive clones.

The generated scFvs are used to generate BCMA-targeted CARs. These BCMA-targeted CARs have similar structure, e.g., each has a transmembrane domain comprising a CD28 polypeptide, and an intracellular domain comprising a CD3 polypeptide and a co-stimulatory signaling region that comprises a CD28 polypeptide. Each of these BCMA-targeted CARs are cloned into a retroviral vector. These viral vectors are then transduced into HEK 293galv9 viral packaging cells in order to generate a stable packaging line for generation of CAR+ T cells. Human T cells (unselected (CD4 and CD8) human T cells from a healthy donor) are transduced with retrovirus in order to express each BCMA-targeted CAR such that the T cells express the BCMA-targeted CARs. The cell surface expression of BCMA-targeted CARs on human T cells is determined via binding A647 conjugated BCMA-Fc fusion protein. The cell surface expression of BCMA-targeted 28z CAR24 is assessed, and cell surface detection is validated by flow cytometry.

The cross-reacting activity of twenty human scFvs between human BCMA and mouse BCMA is assessed. scFvs that cross-react with mouse BCMA are used for syngeneic mouse studies.

### Example 2

### Anti-Tumor Activity of Combined BCMA-Targeted CAR-T Cells and Compound (1) In Vitro

The ability of BCMA-CAR-T cells and Compound (1) to specifically lyse human myeloma cell line (HMCL) is tested. BCMA-CAR-T cells and/or Compound (1) are incubated with GFP expressing tumor cell lines SET2 (Acute myeloid leukemia (AML), CD19-BCMA-); BCWM1 (Lymphoplasmacytic Lymphoma (LPL), CD19-BCMA-); L363 (Multiple Myeloma (MM), CD19-BCMA⁺); NCL-H929; and U266. At time 0, the percent of GFP⁺ tumor line is determined. At 36 h, the BCMA-CAR-T cells and Compound (1) have specifically killed more cells of the GFP⁺ LPL line than either treatment alone. The cytotoxicity was specific to BCMA-expressing cells, as neither the BCMA-CAR-T cells and Compound (1) combination nor each alone lyses BCMA negative CD19 positive Raji Burkett lymphoma cell line.

Drug interaction analysis and the confirmation of synergism is determined by the method of Chou and Talalay. Linear regression analysis of dose-response data is performed using Calcusyn 1.2 software (Biosoft, Cambridge, United Kingdom) to calculate a combination index (CI) for each individual drug combination. To ensure valid statistical analysis, only experimental data for which the linear correlation coefficient of the median-effect plot was more than 0.9 are included. CI values are defined as:

| CI | Definition |
|---|---|
| CI [symbol] 0.3 | strongly synergistic |
| 0.3 < CI [symbol] 0.9 | synergistic |
| 0.9 < CI [symbol] 1.1 | additive |
| 1.1 < CI [symbol] 3.3 | antagonistic |
| 3.3 < CI | strongly antagonistic |

The combination of Compound (1) with BCMA-CAR-T in models of human multiple myeloma in vitro lead to synergistic cell killing. Specifically, Compound (1) when used at concentrations between 10-80 nM (concentrations that are relevant in patients) in combination with BCMA-CAR-T at concentrations of 10⁹ cells produce synergistic cell killing in the U266 and RPMI-8226 human multiple myeloma cell lines.

### Example 3

### Anti-Tumor Activity of Combined BCMA-Targeted CAR-T Cells and Compound (1) In Vivo

BCMA targeted CAR-T cells mediate an anti-myeloma immune response. 1×10⁷ U266 human myeloma cell line cells are injected IV into NSG mice on day 0. On day 4, a) 1×10⁶ BCMA targeted second generation CAR-T cells, Compound (1) or both are injected IV. Imaging of tumors on day 11 (day 7 s/p CAR-T cell injection) shows that the combination treatment mediates an improved anti-tumor response compared to either BCMA targeted CAR-T or Compound (1) treatment alone.

### Example 4

### Anti-Tumor Activity of Combined BCMA-Targeted CAR-T Cells and Compound (1) In Vivo—Human

### Materials and Methods

All patients receive 3 doses of 300 mg/m² cyclophosphamide and 3 doses of 30 mg/m² fludarabine. Chemotherapy is administered because recipient leukocyte depletion enhances the activity of adoptively transferred T cells. Both chemotherapy agents are administered daily on days −5, −4, and −3 before CAR-BCMA T-cell infusion on day 0. CAR-BCMA T cells are administered to each patient with a composition comprising Compound (1). The dose escalation plan calls for an initial dose of 0.3×10⁶ CAR⁺ T cells/kg with threefold increases to each subsequent dose level.

### Follow-Up and Staging

Myeloma staging is conducted according to the International Uniform Response Criteria for Multiple Myeloma. Toxicity is graded by the Common Terminology Criteria for Adverse Events version 4.02. Two weeks, 1 month, 2 months, 3 months, and 6 months after CAR-BCMA infusion, MM is assessed with standard staging tests.

### CAR T-Cell Production

Autologous peripheral blood mononuclear cells (PBMCs) are cultured with an anti-CD3 monoclonal antibody to induce T-cell proliferation. The cells are transduced with the γ-retroviral vector that encodes that CAR, and 9 days after the initiation of cultures, CAR-BCMA T cells are infused.

### Immunologic Assays

CAR-BCMA T cells are detected by flow cytometry after staining with a phycoerythrin-labeled BCMA constant-fragment reagent (PE-BCMA-Fc). CAR-BCMA T cells are also detected by performing quantitative PCR (qPCR). Enzyme-linked immunosorbent assays (ELISA) for interferon γ (IFNγ) are performed on supernatants from cultures of CAR-BCMA T-cell samples plus target cells. Interleukin-6 (IL-6) ELISAs, soluble BCMA ELISAs, and multicytokine assays are performed on patient serum.

### CAR-BCMA Design and CAR T-Cell Production

The anti-BCMA CAR (CAR-BCMA) incorporates the 11D-5-3 anti-BCMA single-chain variable fragment (scFv), a CD28 costimulatory domain, and the CD3-ζ T-cell activation domain. The CAR sequence is expressed by a γ-retroviral vector backbone. CAR-BCMA is consistently expressed on the surface of transduced CD4 and CD8 T cells, and the transduced T cells proliferate extensively in culture. Most infusion T cells express CAR-BCMA. CAR-BCMA T cells specifically recognize BCMA in vitro.

Results demonstrate that the combination treatment mediates an improved anti-tumor response compared to either BCMA targeted CAR-T or Compound (1) treatment alone.

### Example 5

### γ-Secretase Inhibitors Increased B-Cell Maturation Antigen (BCMA) Levels in Lymphoma Cell Lines but not in B-ALL Cell Lines

### Materials and Methods

Cells were seeded at 1×10⁴ cells/well in a total volume of 250 μl into a 96-well plate. The cells were cultured in RPMI supplemented with 10% FCS.

The tested compounds (Compound 1 and Compound 22) were added to the cell cultures at concentrations of: 0, 10, 1, 0.5, 0.1, 0.01, 0.001 μM (0, 1 nM, 10 nM, 100 nM, 500 nM, 1000 nM, and 10000 nM). Following 24 hours of incubation at 37° C., the cells were collected and stained with anti-BCMA antibody and immediately analyzed by FACS.

### Results

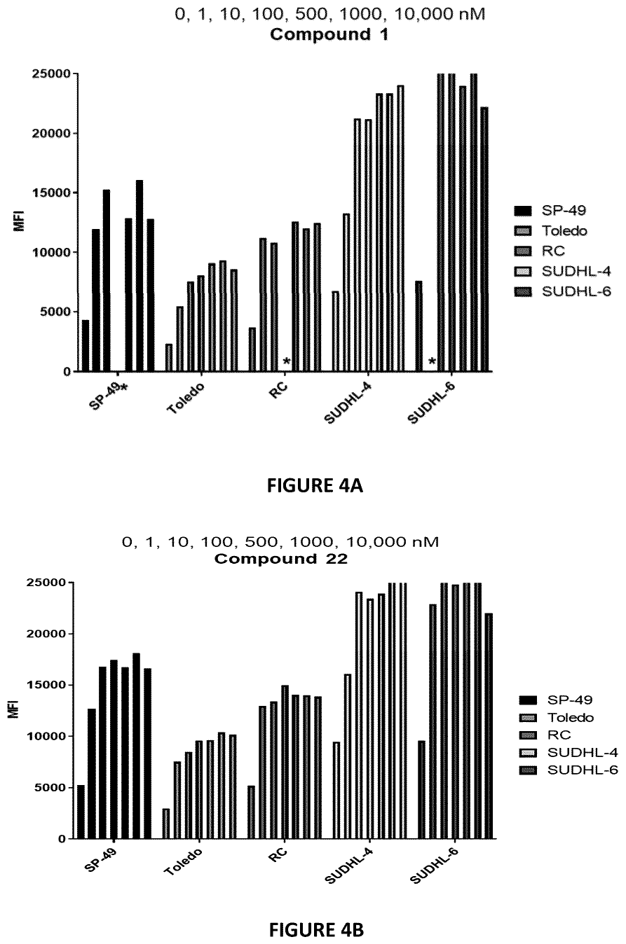

The effect of γ-secretase inhibitors on cell surface BCMA levels in lymphoma cell lines was observed using flow cytometry (FACS analysis) (FIG. 4). Cells from five different lymphoma cell lines were treated for 24 hours with different concentrations of Compound 1 (FIG. 4A) or Compound 22 (FIG. 4B). Lymphoma cell lines treated were: SP49 (MCL with Notch4 GOF); Toledo (DLBCL); RC (Double Hit DLBCL—MYC, BCL2); SUDHL-4 (DLBCL); and SUDHL-6 (DLBCL). GSI concentrations were 0, 1 nM, 10 nM, 100 nM, 500 nM, 1000 nM, and 10000 nM. All lymphoma cell lines had elevated BCMA levels after treatment with 10 nM or more of Compound 1 or Compound 22.

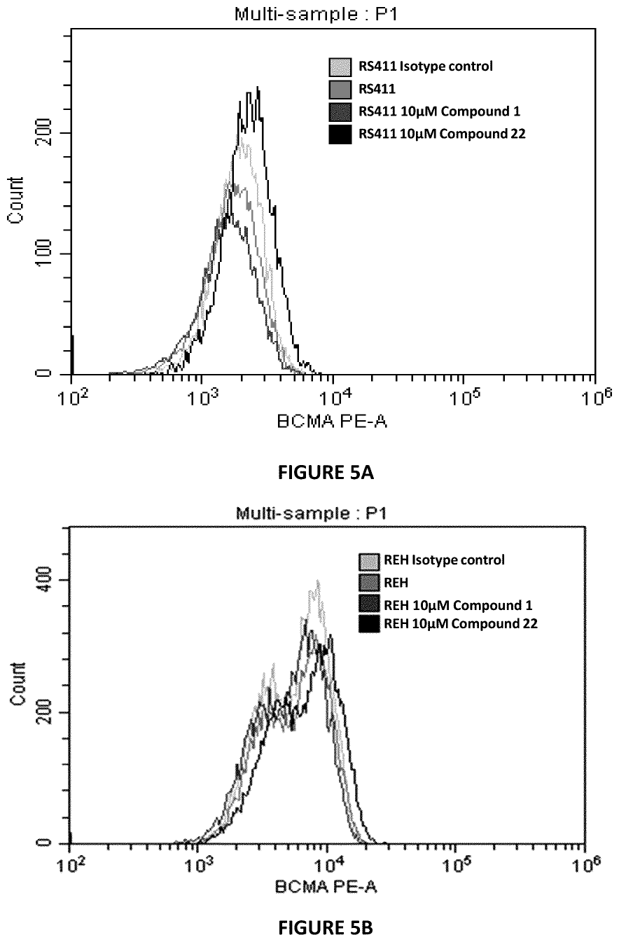

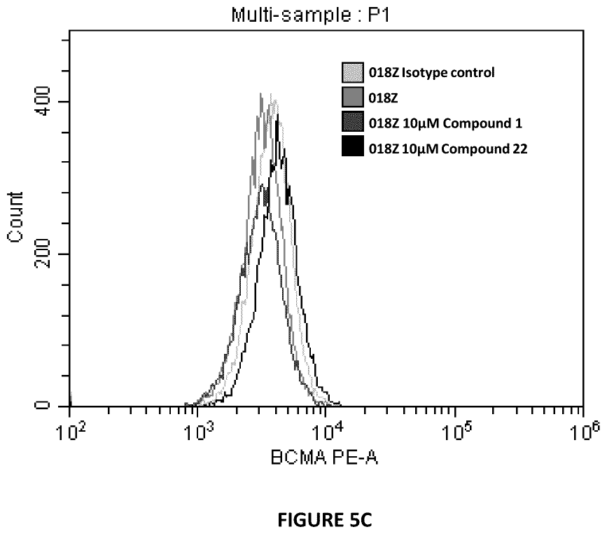

The effect of γ-secretase inhibitors on cell surface BCMA levels in Acute B-Cell Lymphoblastic Leukemia (B-ALL) cell lines was also observed using flow cytometry (FIG. 5). Cells from three different B-ALL cell lines were treated for 24 hours with different concentrations (0-10 μM) of GSIs (Compound 1 and Compound 22). The three B-ALL cell lines, RSV411 (FIG. 5A); REH (FIG. 5B); and 018Z (FIG. 5C), do not express BCMA, and treatment with up to 10 μM GSIs such as Compound 1 or Compound 22 did not affect BCMA expression (FIGS. 5A-5C). Similar results were demonstrated with lower doses of the GSIs (1, 0.5, 0.1, 0.01, 0.001 μM; data not shown).

### Example 6

### γ-Secretase Inhibitors Decreased B-Cell Maturation Antigen (BCMA) Shedding in Lymphoma Cell Lines

### Materials and Methods

The basal cell surface expression of BCMA on lymphoma cells and the effect of γ-secretase inhibitors on a) the expression of BCMA on the cell surface as well as b) the level of soluble BCMA were examined.

38 cell lines, comprising 9 subtypes of lymphomas (see Table 1), were seeded at 1×10⁵ cells/well in a total volume of 250 μl into a 96-well plate. The cells were cultured in RPMI medium supplemented with 10% FCS. Compound (22) was added to the cell cultures at a concentration of 10 μM, 1 μM, 100 nM, 10 nM, 1 nM, 100 μM, 10 μM, 1 μM, and 100 fM, and cell lines (8 from Hadassah Medical Center; 31 from Eurofins) were incubated at 37° C. for 24 hours with Compound (22). Following incubation, the cells were collected and BCMA levels were evaluated on the cell surface for cell-bound BCMA. Cells were stained with an anti-BCMA antibody (PE anti-human 19F2 BCMA, Biolegend), and cell surface BCMA levels were determined by FACS analysis (Mean Fluorescence Intensity. Soluble BCMA levels were analyzed by ELISA (Human BCMA/TNFRSF17 DuoSet kit: R&D Systems).

### Results

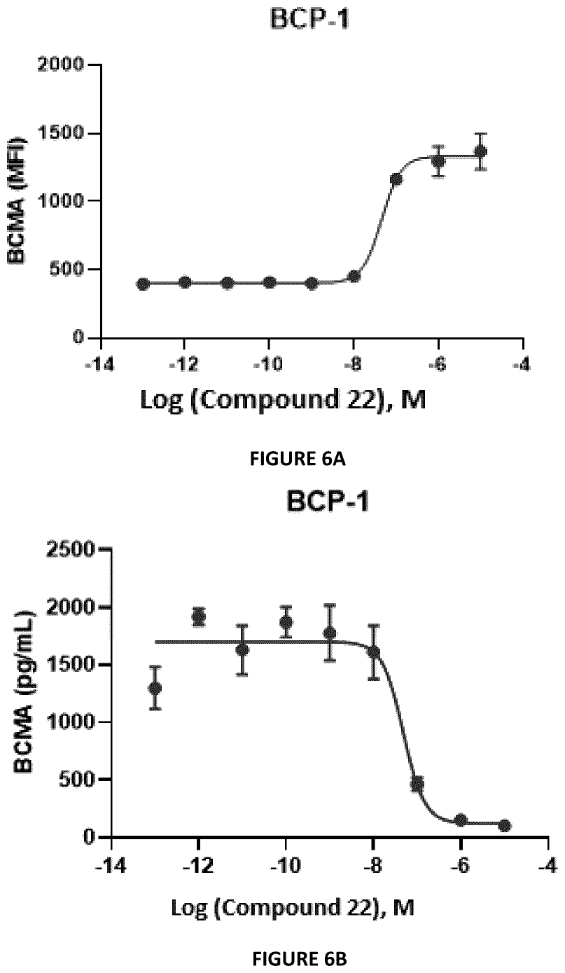

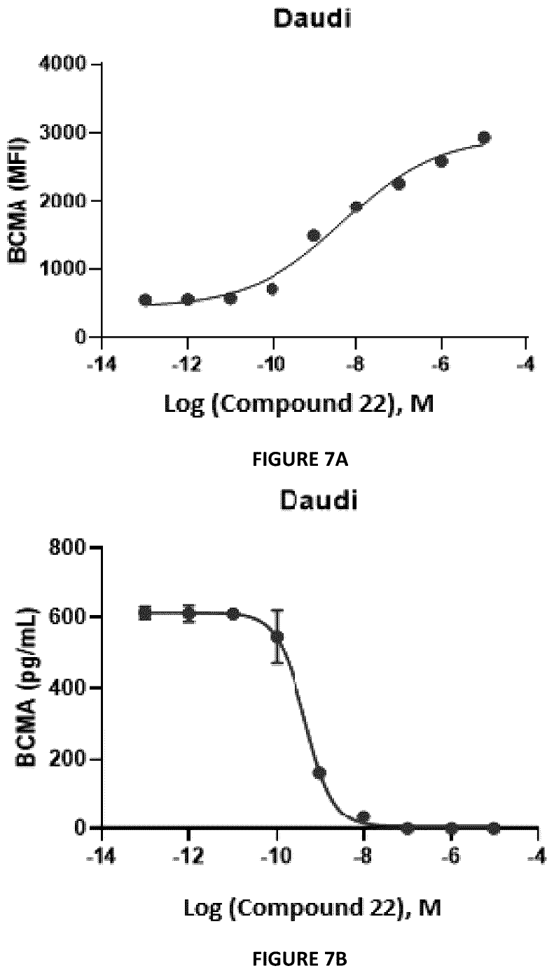

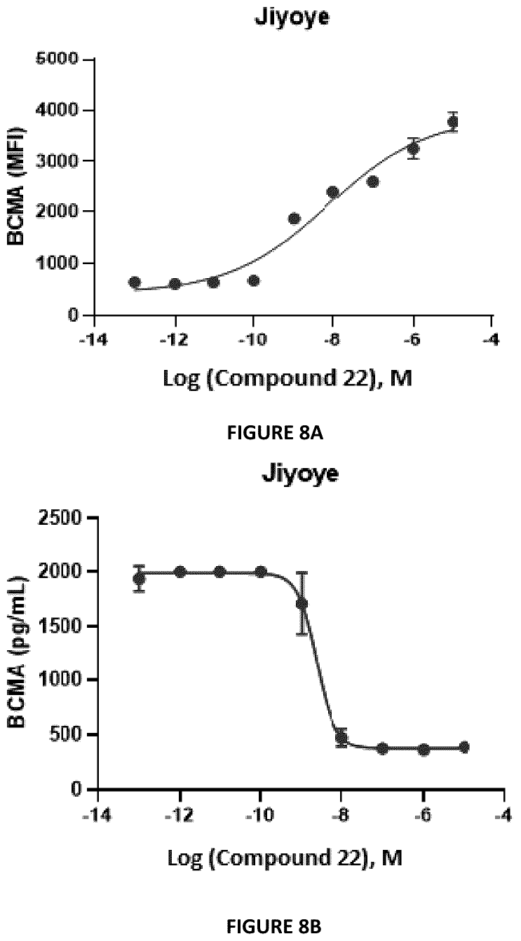

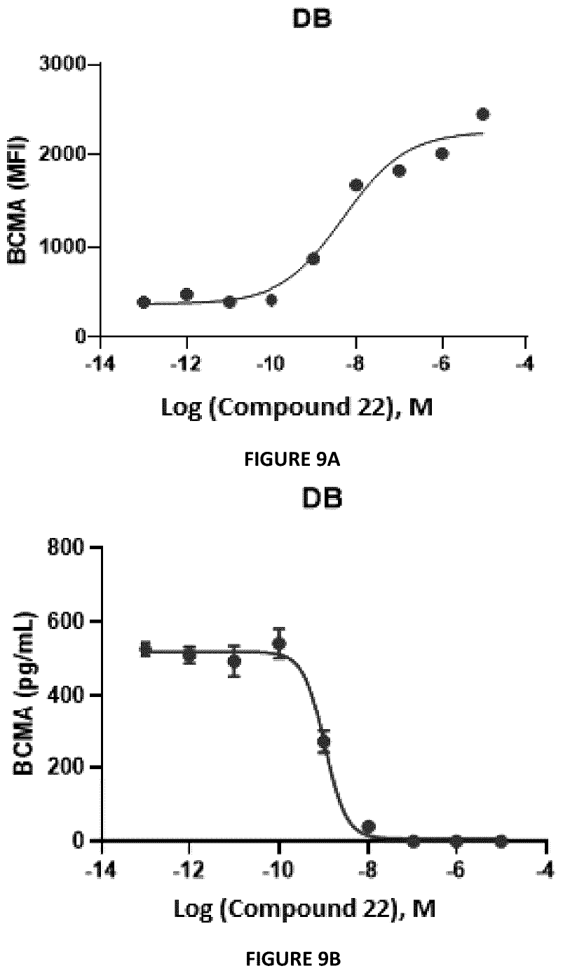

Basal BCMA expression on the cell surface was detected by FACS in all 38 lymphoma cell lines tested (Table 1, GSI concentration 0 nM). Incubation of lymphoma cells with Compound (22) for 24 hours increased cell-bound BCMA levels (Tables 1 and 3 and FIGS. 6A-9A and 10A-S) and reduced soluble BCMA (Tables 2 and 3 and FIG. 6B-9B) in cell lines derived from Mantle Cell Lymphoma, DLBCL, Burkitt's Lymphoma, and B-cell Lymphoma. For example, Compound (22) increased cell-bound BCMA and decreased soluble BCMA in BCP-1 (FIGS. 6A-6B; B-cell lymphoma), Daudi (FIGS. 7A-7B; Burkitt's Lymphoma), Jiyoye (FIGS. 8A-8B; Burkitt's Lymphoma), and DB (FIGS. 9A-9B; DLBCL) cell lines. Compound (22) also increased cell-bound BCMA levels in cell lines from Hodgkin's Lymphoma patients (Table 1). In contrast, Compound (22) did not increase cell-bound BCMA levels in the B-ALL, histiocytic lymphoma, anaplastic large cell lymphoma, or cutaneous T-cell lymphoma cell lines that were tested (Table 1). The data in Table 3 showing that Compound (22) increases mBCMA and decreases shed BCMA in some lymphoma cell lines demonstrates that Compound (22) decreases BCMA shedding from some lymphomas.

TABLE 1
Compound (22) increases BCMA expression
in certain lymphomas by 2-9 fold

| | | BCMA (Mean Fluorescence Intensity) | | | |
|---|---|---|---|---|---|
| | | 0 nM | 1 nM | 100 nM | |
| | | Com- | Com- | Com- | |
| | | pound | pound | pound | EC50 |
| Indication | Cell Line | (22) | (22) | (22) | (nM) |
| B-ALL | RS411 | 2676 | 2273 | 2518 | nt |
| | 018Z | 4763 | 4121 | 4500 | nt |
| | REH | 7239 | 5559 | 6463 | nt |
| Histiocytic | U937 | 315 | 327 | 328 | >10 μM |
| Lymphoma | TUR | 469 | 464 | 462 | >10 μM |
| Anaplastic | SR | 296.5 | 289 | 293 | >10 μM |
| Large Cell | | | | | |
| Lymphoma | | | | | |
| Cutaneous | H9 | 844 | 811 | 900 | >10 μM |
| T-cell | | | | | |
| Lymphoma | | | | | |
| Hodgkin's | RPMI-6666 | 497 | 971.5 | 3287.5 | 7.0 |
| Lymphoma | L-248 | 449 | 440 | 445 | >10 μM |
| Mantle Cell | Jeko-1 | 389 | 1654 | 2885 | 1.2 |
| Lymphoma | JVM-2 | 3118 | 4437 | 21757 | 3.1 |
| | SP49 | 5276 | 12697 | 17462 | nt |
| | REC-1 | 234 | 217 | 243 | >10 μM |
| DLBCL | ST486 | 2997 | 10564 | 13410 | 0.58 |
| | DOHH-2 | 2848 | 8033 | 12620 | 0.87 |
| | SUDHL | 2268 | 7568 | 20540 | 1.6 |
| | HT | 282 | 488 | 895 | 2.3 |
| | DB | 450 | 870 | 1827 | 4.9 |
| | SUDHL-10 | 1161 | 6061 | 7822 | 5.9 |
| | Toledo | 2978 | 7541 | 9580 | nt |
| | RC | 5185 | 12970 | 15001 | nt |
| | SUDHL-4 | 9481 | 16100 | 23438 | nt |
| | SUDHL-6 | 9569 | 22915 | 24812 | nt |
| | SUDHL-8 | 238 | 350 | 414 | >10 μM |
| Burkitt's | GA-10 | 9196 | 41993 | 51080 | 0.56 |
| Lymphoma | EB-2 | 5267 | 18536 | 28188 | 0.98 |
| | Raji | 290 | 521 | 650 | 1.1 |
| | Ramos | 949 | 4899 | 10264 | 1.4 |
| | CA46 | 2457 | 6424 | 13506 | 2.3 |
| | Daudi | 532 | 1421 | 2242 | 4.9 |
| | Jiyoye | 621 | 1864 | 2616 | 7.6 |
| | Namalwa | 843 | 1395 | 1690 | 10.7 |
| | EB-3 | 824 | 549 | 449 | >10 μM |
| B-cell | NU-DUL-1 | 671 | 3892 | 4665 | 0.61 |
| Lymphoma | MHHH-PREB-1 | 408 | 1363 | 1830 | 1.7 |
| | MC-116 | 438 | 915 | 1850 | 1.9 |
| | CRO-AP2 | 728 | 855 | 4782 | 5.6 |
| | BC-1 | 625 | 676 | 3694 | 30.6 |
| | BCP-1 | 405 | 398 | 1159 | 45.6 |

TABLE 2
Compound (22) decreases soluble BCMA
in some Lymphoma/Leukemia cell lines

| | | 0 nM | 1 nM | 10 nM | 100 nM |
|---|---|---|---|---|---|
| | | Com- | Com- | Com- | Com- |
| | | pound | pound | pound | pound |
| Indication | Cell Line | (22) | (22) | (22) | (22) |
| B-Cell | BC-1 | 1187.3 | >2000 | 1841.6 | 878.6 |
| Lymphoma | BCP-1 | 1754.8 | 1665.7 | 1610 | 460.6 |
| | CRO-AP2 | 970.9 | 1895.3 | 409.5 | 126.1 |
| | MHHH-PREB-1 | 1331.7 | 227.3 | 75.4 | 60.1 |
| | MC116 | 1453.9 | 1810.6 | 414.8 | 303.7 |
| Mantle | JEKO-1 | 1277.1 | 1245.6 | 338 | 305.6 |
| Cell | | | | | |
| Lymphoma | | | | | |
| Burkitt's | NAMALWA | 1441.4 | 87.5 | 66.6 | 67.4 |
| Lymphoma | CA-46 | 1718.7 | 1775.4 | 330 | 173.2 |
| | Daudi | 580.2 | 162.6 | 36.8 | 0 |
| | JIYOYE | 1567.2 | 1707.7 | 472.3 | 373.1 |
| | EB2 | 1688.2 | >2000 | 597.7 | 479.4 |
| | GA-10 | >2000 | >2000 | 800.8 | 398.1 |
| | Raji | 575.6 | 81.4 | 29.7 | 26.3 |
| | Ramos | 1000.7 | >2000 | 613.3 | 504.3 |
| DLBCL | DOHH-2 | 1404.7 | 1272.1 | 136 | 98.4 |
| | DB | 546.9 | 270.5 | 40.5 | 0 |
| | HT | 802.1 | 389.4 | 99.4 | 38 |

TABLE 3
Compound (22) increases mBCMA & abrogates
shedding in certain lymphomas

| | | mBCMA (MFI) | | Shed BCMA (pg/ml) | |
|---|---|---|---|---|---|
| | | Fold | EC₅₀ | % | EC₅₀ |
| Indication | Cell Line | Increase | (nM) | Reduction | (nM) |
| B-ALL | RS411 | 0.9 | nt | nt | nt |
| | 018Z | 0.9 | nt | nt | nt |
| | REH | 0.9 | nt | nt | nt |
| Histiocytic | U937 | 1.0 | >10 mM | BLQ | >10 mM |
| Lymphoma | TUR | 1.0 | >10 mM | BLQ | >10 mM |
| Anaplastic | SR | 1.0 | >10 mM | BLQ | >10 mM |
| Large Cell | | | | | |
| Lymphoma | | | | | |
| Cutaneous | H9 | 1.1 | >10 mM | BLQ | >10 mM |
| T-cell | | | | | |
| Lymphoma | | | | | |
| Hodgkin's | RPMI-6666 | 6.6 | 7.0 | 92% | 9.1 |
| Lymphoma | L-248 | 1.0 | >10 mM | BLQ | >10 mM |
| Mantle Cell | Jeko-1 | 7.4 | 1.2 | 76% | 1.1 |
| Lymphoma | JVM-2 | 7.0 | 3.1 | 57% | 75.3 |
| | SP49 | 3.3 | nt | nt | nt |
| | REC-1 | 1.0 | >10 mM | BLQ | >10 mM |
| DLBCL | ST486 | 4.5 | 0.58 | 90 | 0.9 |
| | DOHH-2 | 4.4 | 0.87 | 93 | 2.2 |
| | HT | 3.2 | 2.3 | 98 | 1 |
| | DB | 4.1 | 4.9 | 100 | 1 |
| | Toledo | 3.2 | nt | nt | nt |
| | RC | 2.9 | nt | nt | nt |
| | SUDHL-4 | 2.5 | nt | nt | nt |
| | (Haddasah) | | | | |
| | SUDHL-4 | 9.1 | 1.6 | 87 | 6.6 |
| | (Eurofins) | | | | |
| | SUDHL-6 | 2.6 | nt | nt | nt |
| | SUDHL-8 | 1.7 | >10 mM | 100 | 0.5 |
| | SUDHL-10 | 6.7 | 5.9 | 97 | 0.9 |
| Burkitt's | GA-10 | 5.6 | 0.56 | 83 | 7.4 |
| Lymphoma | EB-2 | 5.4 | 0.98 | 70 | 7.1 |
| | Raji | 2.2 | 1.1 | 99 | 0.3 |
| | Ramos | 10.8 | 1.4 | 76 | 8.6 |
| | CA46 | 5.5 | 2.3 | 90 | 7.4 |
| | Daudi | 4.2 | 4.9 | 100 | 0.5 |
| | Jiyoye | 4.2 | 7.6 | 81 | 2.3 |
| | Namalwa | 2.0 | 10.7 | 95 | 0.6 |
| | EB-3 | 0.5 | >10 mM | BLQ | >10 mM |
| B-cell | NU-DUL-1 | 7.0 | 0.61 | 91 | 1 |
| Lymphoma | MHHH-PREB-1 | 4.5 | 1.7 | 97 | 0.6 |
| | MC-116 | 4.2 | 1.9 | 76 | 6.5 |
| | CRO-AP2 | 6.6 | 5.6 | 91 | 4.8 |
| | BC-1 | 5.9 | 30.6 | 30 | 84 |
| | BCP-1 | 2.9 | 45.6 | 94 | 48 |

BCMA is a protein that is not highly expressed on lymphoma cells. BCMA is actively shed from lymphoma cells, a process which is mediated by gamma secretase. It was demonstrated herein that Compound (22) inhibits BCMA shedding (e.g., increases BCMA levels on the target lymphoma cells and decreases soluble BCMA levels). Since soluble BCMA can sequester anti-BCMA therapies, such as BCMA CAR-T and anti-BCMA bi-specific monoclonal antibodies and hamper treatment, GSJs that inhibit BCMA shedding such as Compound (22) are good candidates for combination with anti-BCMA therapies.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method of improving the efficacy of a BCMA-targeting immuno-therapeutic or treating, suppressing or inhibiting multiple myeloma in a subject, comprising the step of administering to said subject a first composition comprising one or more compounds represented by the structure of Formula (I): 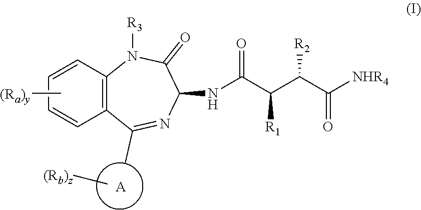
and/or at least one salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rₓ;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂, 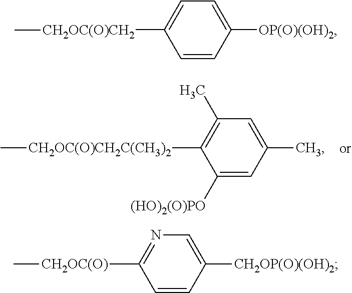
R_{y} is: —SCH₂CH(NH₂)C(O)OH, —SCH₂CH(NH₂)C(O)OCH₃, or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is 1 or 2,
and a second composition comprising one or more BCMA-targeting immuno-therapeutics.

2. The method of claim 1, wherein said BCMA-targeting immunotherapeutic comprises chimeric antigen receptor T (CAR-T) cells, a bispecific antibody, an antibody drug conjugate, a tri-specific antibody, a trifunctional antibody, chemically linked Fab, or a bi-specific T-cell engager (BiTE).

3. The method of claim 1, wherein said first composition and/or said second composition is administered intravenously or orally to said subject.

4. The method of claim 1, wherein said first composition and said second composition are administered together or said first composition and said second composition are administered at separate sites or at separate times.

5. The method of claim 1, wherein said composition comprising Formula (I) is administered prior to and then again subsequent to the administration of said composition comprising said one or more B-cell maturation antigen (BCMA)-targeting immuno-therapeutics.

6. The method of claim 1, wherein said one or more compounds of Formula (I) are represented by the structure of Formula (II): 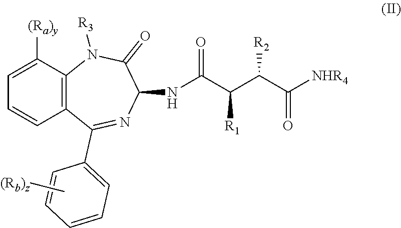
wherein
R₃ is H or —CH₃; and
y is zero or 1.

7. The method of claim 6, wherein said one or more compounds is represented by the structure of Formula (IV):

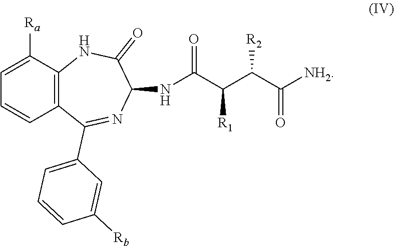

8. The method of claim 6, wherein said one or more compounds is represented by the structure of Formula (V): 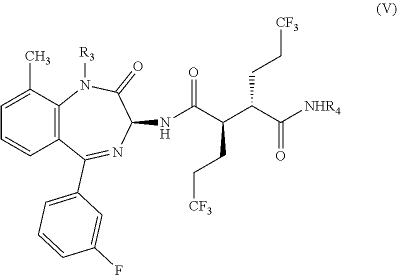
wherein R₃ is H or Rₓ.

9. The method of claim 6, wherein said one or more compounds are selected from: (2R,3S)—N-((3S)-5-(3-fluorophenyl)-9-methyl-2-oxo-2,3-dihydro-1H-1,4-benzodiazepin-3-yl)-2,3-bis(3,3,3-trifluoropropyl)succinamide (22); (2R,3S)—N-((3S)-5-(3-chlorophenyl)-9-ethyl-2-oxo-2,3-dihydro-1H-1,4-benzodiazepin-3-yl)-2,3-bis(3,3,3-trifluoropropyl)succinamide (23); (2R,3S)—N-((3S)-5-(3-chlorophenyl)-9-isopropyl-2-oxo-2,3-dihydro-1H-1,4-benzodiazepin-3-yl)-2,3-bis(3,3,3-trifluoropropyl)succinamide (24); (2R,3S)—N-(9-chloro-5-(3,4-dimethylphenyl)-2-oxo-2,3-dihydro-1H-1,4-benzodiazepin-3-yl)-3-(4,4,4-trifluorobutyl)-2-(3,3,3-trifluoropropyl)succinamide (25); (2R,3S)—N-(9-chloro-5-(3,5-dimethylphenyl)-2-oxo-2,3-dihydro-1H-1,4-benzodiazepin-3-yl)-3-(4,4,4-trifluorobutyl)-2-(3,3,3-trifluoropropyl)succinamide (26); (2R,3S)—N-((3S)-9-ethyl-5-(3-methylphenyl)-2-oxo-2,3-dihydro-1H-1,4-benzodiazepin-3-yl)-2,3-bis(3,3,3-trifluoropropyl)succinamide (27); (2R,3S)—N-((3S)-5-(3-chlorophenyl)-9-methyl-2-oxo-2,3-dihydro-1H-1,4-benzodiazepin-3-yl)-2,3-bis(3,3,3-trifluoropropyl)succinamide (28); (2R,3S)—N-((3S)-5-(3-chlorophenyl)-9-methyl-2-oxo-2,3-dihydro-1H-1,4-benzodiazepin-3-yl)-3-(4,4,4-trifluorobutyl)-2-(3,3,3-trifluoropropyl)succinamide (29); (2R,3S)—N-((3S)-5-(3-methylphenyl)-2-oxo-9-(trifluoromethyl)-2,3-dihydro-1H-1,4-benzodiazepin-3-yl)-2,3-bis(3,3,3-trifluoropropyl)succinamide (30); (2R,3S)—N-((3S)-9-chloro-5-(3,5-dimethylphenyl)-2-oxo-2,3-dihydro-1H-1,4-benzodiazepin-3-yl)-2,3-bis(3,3,3-trifluoropropyl)succinamide (31); (2R,3S)—N-((3S)-5-(3-methylphenyl)-2-oxo-9-(trifluoromethyl)-2,3-dihydro-1H-1,4-benzodiazepin-3-yl)-3-(4,4,4-trifluorobutyl)-2-(3,3,3-trifluoropropyl)succinamide (32); (2R,3S)—N-((3S)-9-isopropyl-5-(3-methylphenyl)-2-oxo-2,3-dihydro-1H-1,4-benzodiazepin-3-yl)-2,3-bis(3,3,3-trifluoropropyl)succinamide (33); (2R,3S)—N-((3S)-9-(cyclopropyloxy)-5-(3-methylphenyl)-2-oxo-2,3-dihydro-1H-1,4-benzodiazepin-3-yl)-3-(4,4,4-trifluorobutyl)-2-(3,3,3-trifluoropropyl)succinamide (34); (2R,3S)—N-((3S)-9-(cyclopropyloxy)-5-(3-methylphenyl)-2-oxo-2,3-dihydro-1H-1,4-benzodiazepin-3-yl)-2,3-bis(3,3,3-trifluoropropyl)succinamide (35); (2R,3S)—N-((3S)-9-chloro-5-(3-methylphenyl)-2-oxo-2,3-dihydro-1H-1,4-benzodiazepin-3-yl)-3-(4,4,4-trifluorobutyl)-2-(3,3,3-trifluoropropyl) succinamide (36); (2R,3S)—N-((3S)-9-methyl-2-oxo-5-(3-(trifluoromethyl)phenyl)-2,3-dihydro-1H-1,4-benzodiazepin-3-yl)-3-(4,4,4-trifluorobutyl)-2-(3,3,3-trifluoropropyl) succinamide (37); (2R,3S)—N-((3S)-9-methyl-2-oxo-5-(3-(trifluoromethyl) phenyl)-2,3-dihydro-1H-1,4-benzodiazepin-3-yl)-2,3-bis(3,3,3-trifluoropropyl) succinamide (38); (2R,3S)—N-((3S)-9-chloro-5-(2-methylphenyl)-2-oxo-2,3-dihydro-1H-1,4-benzodiazepin-3-yl)-2,3-bis(3,3,3-trifluoropropyl)succinamide (39); (2R,3S)—N-((3S)-5-(4-fluorophenyl)-9-methyl-2-oxo-2,3-dihydro-1H-1,4-benzodiazepin-3-yl)-2,3-bis(3,3,3-trifluoropropyl)succinamide (40); (2R,3S)—N-((3S)-9-chloro-5-(3-cyclopropylphenyl)-2-oxo-2,3-dihydro-1H-1,4-benzodiazepin-3-yl)-2,3-bis(3,3,3-trifluoropropyl)succinamide (41); (2R,3S)—N-((3S)-5-(3-chlorophenyl)-9-methoxy-2-oxo-2,3-dihydro-1H-1,4-benzodiazepin-3-yl)-2,3-bis(3,3,3-trifluoropropyl)succinamide (42); (2R,3S)—N-((3S)-5-(4-chlorophenyl)-9-methoxy-2-oxo-2,3-dihydro-1H-1,4-benzodiazepin-3-yl)-2,3-bis(3,3,3-trifluoropropyl)succinamide (43); (2R,3S)—N-((3S)-9-chloro-5-(3-methylphenyl)-2-oxo-2,3-dihydro-1H-1,4-benzodiazepin-3-yl)-2,3-bis(3,3,3-trifluoropropyl)succinamide (44); (2R,3S)—N-((3S)-5-(3-methylphenyl)-9-methoxy-2-oxo-2,3-dihydro-1H-1,4-benzodiazepin-3-yl)-2,3-bis(3,3,3-trifluoropropyl)succinamide (45); (2R,3S)—N-((3S)-5-(4-(hydroxymethyl)phenyl)-2-oxo-2,3-dihydro-1H-1,4-benzodiazepin-3-yl)-2,3-bis(3,3,3-trifluoropropyl)succinamide (46); (2R,3S)—N-((3S)-5-(2-methylphenyl)-2-oxo-2,3-dihydro-1H-1,4-benzodiazepin-3-yl)-2,3-bis(3,3,3-trifluoropropyl)succinamide (47); (2R,3S)—N-((3S)-5-(3-methylphenyl)-2-oxo-2,3-dihydro-1H-1,4-benzodiazepin-3-yl)-2,3-bis(3,3,3-trifluoropropyl)succinamide (48); (2R,3S)—N-((3S)-9-methoxy-2-oxo-5-(5-(trifluoromethyl)-2-pyridinyl)-2,3-dihydro-1H-1,4-benzodiazepin-3-yl)-2,3-bis(3,3,3-trifluoropropyl)succinamide (49); (2R,3S)—N-((3S)-5-(5-chloro-2-pyridinyl)-9-methoxy-2-oxo-2,3-dihydro-1H-1,4-benzodiazepin-3-yl)-2,3-bis(3,3,3-trifluoropropyl)succinamide (50); (2R,3S)—N-((3S)-5-(4-methoxyphenyl)-2-oxo-2,3-dihydro-1H-1,4-benzodiazepin-3-yl)-2,3-bis(3,3,3-trifluoropropyl)succinamide (51); (2R,3S)—N-((3S)-5-(4-methylphenyl)-2-oxo-2,3-dihydro-1H-1,4-benzodiazepin-3-yl)-2,3-bis(3,3,3-trifluoropropyl)succinamide (52); (2R,3S)—N-((3S)-5-(3-fluorophenyl)-9-(hydroxymethyl)-2-oxo-2,3-dihydro-1H-1,4-benzodiazepin-3-yl)-2,3-bis(3,3,3-trifluoropropyl)succinamide (53); ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl L-valinate (54); ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl L-alaninate (55); S-(((2S,3R)-6,6,6-trifluoro-3-(((3S)-5-(3-fluorophenyl)-9-methyl-2-oxo-2,3-dihydro-1H-1,4-benzodiazepin-3-yl)carbamoyl)-2-(3,3,3-trifluoropropyl)hexanoyl)amino)-L-cysteine (56); tert-butyl S-(((2S,3R)-6,6,6-trifluoro-3-(((3S)-5-(3-fluorophenyl)-9-methyl-2-oxo-2,3-dihydro-1H-1,4-benzodiazepin-3-yl)carbamoyl)-2-(3,3,3-trifluoropropyl)hexanoyl)amino)-L-cysteinate (57); methyl S-(((2S,3R)-6,6,6-trifluoro-3-(((3S)-5-(3-fluorophenyl)-9-methyl-2-oxo-2,3-dihydro-1H-1,4-benzodiazepin-3-yl)carbamoyl)-2-(3,3,3-trifluoropropyl) hexanoyl)amino)-L-cysteinate (58); ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl (4-(phosphonooxy)phenyl)acetate (59); ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl L-valyl-L-valinate (60); and salts thereof.

10. The method of claim 6, wherein said compound comprises:

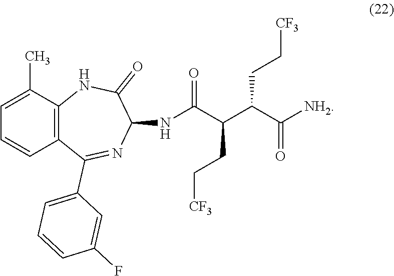 [SMILES: Cc1cccc2c1NC(=O)[C@@H](NC(=O)[C@H](CCC(F)(F)F)[C@H](CCC(F)(F)F)C(N)=O)N=C2c1cccc(F)c1]

11. The method of claim 1, wherein said multiple myeloma comprises cells with low or undetectable levels of BCMA, or BCMA levels are under the threshold for allowing anti-BCMA therapies to be effective, and wherein said BCMA comprises BCMA expressed on the cell surface.

12. A method of decreasing B-cell maturation antigen (BCMA) shedding or decreasing soluble BCMA in a multiple myeloma cell in a subject having multiple myeloma comprising the step of administering to said subject a composition comprising one or more compounds represented by the structure of Formula (I): 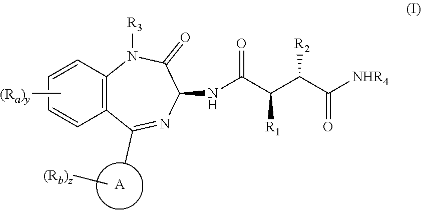
and/or at least one salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rₓ;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂, 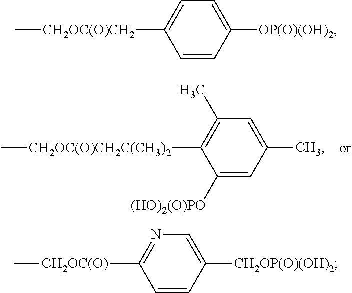
R_{y} is: —SCH₂CH(NH₂)C(O)OH, —SCH₂CH(NH₂)C(O)OCH₃, or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is 1 or 2.

13. The method of claim 12, wherein said composition is administered intravenously or orally to said subject.

14. The method of claim 12, wherein said one or more compounds of Formula (I) are represented by the structure of Formula (II): 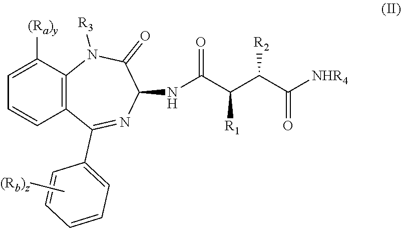
wherein
R₃ is H or —CH₃; and
y is zero or 1.

15. The method of claim 12, wherein said one or more compounds is represented by the structure of Formula (IV):

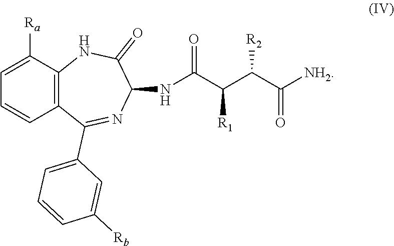

16. The method of claim 12, wherein said one or more compounds is represented by the structure of Formula (V): 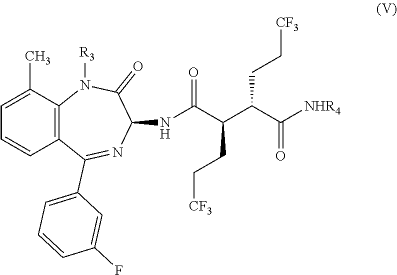
wherein R₃ is H or Rₓ.

17. The method of claim 12, wherein said one or more compounds are selected from: (2R,3S)—N-((3S)-5-(3-fluorophenyl)-9-methyl-2-oxo-2,3-dihydro-1H-1,4-benzodiazepin-3-yl)-2,3-bis(3,3,3-trifluoropropyl)succinamide (22); (2R,3S)—N-((3S)-5-(3-chlorophenyl)-9-ethyl-2-oxo-2,3-dihydro-1H-1,4-benzodiazepin-3-yl)-2,3-bis(3,3,3-trifluoropropyl)succinamide (23); (2R,3S)—N-((3S)-5-(3-chlorophenyl)-9-isopropyl-2-oxo-2,3-dihydro-1H-1,4-benzodiazepin-3-yl)-2,3-bis(3,3,3-trifluoropropyl)succinamide (24); (2R,3S)—N-(9-chloro-5-(3,4-dimethylphenyl)-2-oxo-2,3-dihydro-1H-1,4-benzodiazepin-3-yl)-3-(4,4,4-trifluorobutyl)-2-(3,3,3-trifluoropropyl)succinamide (25); (2R,3S)—N-(9-chloro-5-(3,5-dimethylphenyl)-2-oxo-2,3-dihydro-1H-1,4-benzodiazepin-3-yl)-3-(4,4,4-trifluorobutyl)-2-(3,3,3-trifluoropropyl)succinamide (26); (2R,3S)—N-((3S)-9-ethyl-5-(3-methylphenyl)-2-oxo-2,3-dihydro-1H-1,4-benzodiazepin-3-yl)-2,3-bis(3,3,3-trifluoropropyl)succinamide (27); (2R,3S)—N-((3S)-5-(3-chlorophenyl)-9-methyl-2-oxo-2,3-dihydro-1H-1,4-benzodiazepin-3-yl)-2,3-bis(3,3,3-trifluoropropyl)succinamide (28); (2R,3S)—N-((3S)-5-(3-chlorophenyl)-9-methyl-2-oxo-2,3-dihydro-1H-1,4-benzodiazepin-3-yl)-3-(4,4,4-trifluorobutyl)-2-(3,3,3-trifluoropropyl)succinamide (29); (2R,3S)—N-((3S)-5-(3-methylphenyl)-2-oxo-9-(trifluoromethyl)-2,3-dihydro-1H-1,4-benzodiazepin-3-yl)-2,3-bis(3,3,3-trifluoropropyl)succinamide (30); (2R,3S)—N-((3S)-9-chloro-5-(3,5-dimethylphenyl)-2-oxo-2,3-dihydro-1H-1,4-benzodiazepin-3-yl)-2,3-bis(3,3,3-trifluoropropyl)succinamide (31); (2R,3S)—N-((3S)-5-(3-methylphenyl)-2-oxo-9-(trifluoromethyl)-2,3-dihydro-1H-1,4-benzodiazepin-3-yl)-3-(4,4,4-trifluorobutyl)-2-(3,3,3-trifluoropropyl)succinamide (32); (2R,3S)—N-((3S)-9-isopropyl-5-(3-methylphenyl)-2-oxo-2,3-dihydro-1H-1,4-benzodiazepin-3-yl)-2,3-bis(3,3,3-trifluoropropyl)succinamide (33); (2R,3S)—N-((3S)-9-(cyclopropyloxy)-5-(3-methylphenyl)-2-oxo-2,3-dihydro-1H-1,4-benzodiazepin-3-yl)-3-(4,4,4-trifluorobutyl)-2-(3,3,3-trifluoropropyl)succinamide (34); (2R,3S)—N-((3S)-9-(cyclopropyloxy)-5-(3-methylphenyl)-2-oxo-2,3-dihydro-1H-1,4-benzodiazepin-3-yl)-2,3-bis(3,3,3-trifluoropropyl)succinamide (35); (2R,3S)—N-((3S)-9-chloro-5-(3-methylphenyl)-2-oxo-2,3-dihydro-1H-1,4-benzodiazepin-3-yl)-3-(4,4,4-trifluorobutyl)-2-(3,3,3-trifluoropropyl) succinamide (36); (2R,3S)—N-((3S)-9-methyl-2-oxo-5-(3-(trifluoromethyl)phenyl)-2,3-dihydro-1H-1,4-benzodiazepin-3-yl)-3-(4,4,4-trifluorobutyl)-2-(3,3,3-trifluoropropyl) succinamide (37); (2R,3S)—N-((3S)-9-methyl-2-oxo-5-(3-(trifluoromethyl) phenyl)-2,3-dihydro-1H-1,4-benzodiazepin-3-yl)-2,3-bis(3,3,3-trifluoropropyl) succinamide (38); (2R,3S)—N-((3S)-9-chloro-5-(2-methylphenyl)-2-oxo-2,3-dihydro-1H-1,4-benzodiazepin-3-yl)-2,3-bis(3,3,3-trifluoropropyl)succinamide (39); (2R,3S)—N-((3S)-5-(4-fluorophenyl)-9-methyl-2-oxo-2,3-dihydro-1H-1,4-benzodiazepin-3-yl)-2,3-bis(3,3,3-trifluoropropyl)succinamide (40); (2R,3S)—N-((3S)-9-chloro-5-(3-cyclopropylphenyl)-2-oxo-2,3-dihydro-1H-1,4-benzodiazepin-3-yl)-2,3-bis(3,3,3-trifluoropropyl)succinamide (41); (2R,3S)—N-((3S)-5-(3-chlorophenyl)-9-methoxy-2-oxo-2,3-dihydro-1H-1,4-benzodiazepin-3-yl)-2,3-bis(3,3,3-trifluoropropyl)succinamide (42); (2R,3S)—N-((3S)-5-(4-chlorophenyl)-9-methoxy-2-oxo-2,3-dihydro-1H-1,4-benzodiazepin-3-yl)-2,3-bis(3,3,3-trifluoropropyl)succinamide (43); (2R,3S)—N-((3S)-9-chloro-5-(3-methylphenyl)-2-oxo-2,3-dihydro-1H-1,4-benzodiazepin-3-yl)-2,3-bis(3,3,3-trifluoropropyl)succinamide (44); (2R,3S)—N-((3S)-5-(3-methylphenyl)-9-methoxy-2-oxo-2,3-dihydro-1H-1,4-benzodiazepin-3-yl)-2,3-bis(3,3,3-trifluoropropyl)succinamide (45); (2R,3S)—N-((3S)-5-(4-(hydroxymethyl)phenyl)-2-oxo-2,3-dihydro-1H-1,4-benzodiazepin-3-yl)-2,3-bis(3,3,3-trifluoropropyl)succinamide (46); (2R,3S)—N-((3S)-5-(2-methylphenyl)-2-oxo-2,3-dihydro-1H-1,4-benzodiazepin-3-yl)-2,3-bis(3,3,3-trifluoropropyl)succinamide (47); (2R,3S)—N-((3S)-5-(3-methylphenyl)-2-oxo-2,3-dihydro-1H-1,4-benzodiazepin-3-yl)-2,3-bis(3,3,3-trifluoropropyl)succinamide (48); (2R,3S)—N-((3S)-9-methoxy-2-oxo-5-(5-(trifluoromethyl)-2-pyridinyl)-2,3-dihydro-1H-1,4-benzodiazepin-3-yl)-2,3-bis(3,3,3-trifluoropropyl)succinamide (49); (2R,3S)—N-((3S)-5-(5-chloro-2-pyridinyl)-9-methoxy-2-oxo-2,3-dihydro-1H-1,4-benzodiazepin-3-yl)-2,3-bis(3,3,3-trifluoropropyl)succinamide (50); (2R,3S)—N-((3S)-5-(4-methoxyphenyl)-2-oxo-2,3-dihydro-1H-1,4-benzodiazepin-3-yl)-2,3-bis(3,3,3-trifluoropropyl)succinamide (51); (2R,3S)—N-((3S)-5-(4-methylphenyl)-2-oxo-2,3-dihydro-1H-1,4-benzodiazepin-3-yl)-2,3-bis(3,3,3-trifluoropropyl)succinamide (52); (2R,3S)—N-((3S)-5-(3-fluorophenyl)-9-(hydroxymethyl)-2-oxo-2,3-dihydro-1H-1,4-benzodiazepin-3-yl)-2,3-bis(3,3,3-trifluoropropyl)succinamide (53); ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl L-valinate (54); ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl L-alaninate (55); S-(((2S,3R)-6,6,6-trifluoro-3-(((3S)-5-(3-fluorophenyl)-9-methyl-2-oxo-2,3-dihydro-1H-1,4-benzodiazepin-3-yl)carbamoyl)-2-(3,3,3-trifluoropropyl)hexanoyl)amino)-L-cysteine (56); tert-butyl S-(((2S,3R)-6,6,6-trifluoro-3-(((3S)-5-(3-fluorophenyl)-9-methyl-2-oxo-2,3-dihydro-1H-1,4-benzodiazepin-3-yl)carbamoyl)-2-(3,3,3-trifluoropropyl)hexanoyl)amino)-L-cysteinate (57); methyl S-(((2S,3R)-6,6,6-trifluoro-3-(((3S)-5-(3-fluorophenyl)-9-methyl-2-oxo-2,3-dihydro-1H-1,4-benzodiazepin-3-yl)carbamoyl)-2-(3,3,3-trifluoropropyl) hexanoyl)amino)-L-cysteinate (58); ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl (4-(phosphonooxy)phenyl)acetate (59); ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl L-valyl-L-valinate (60); and salts thereof.

18. The method of claim 12, wherein said compound comprises:

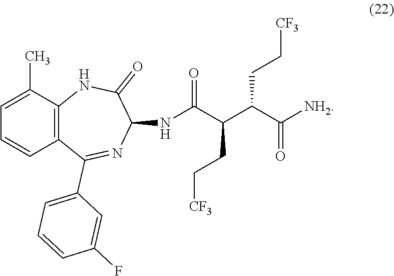 [SMILES: Cc1cccc2c1NC(=O)[C@@H](NC(=O)[C@H](CCC(F)(F)F)[C@H](CCC(F)(F)F)C(N)=O)N=C2c1cccc(F)c1]

19. The method of claim 13, wherein said multiple myeloma comprises cells with low or undetectable levels of BCMA, or BCMA levels are under the threshold for allowing anti-BCMA therapies to be effective, and wherein said BCMA comprises BCMA expressed on the cell surface.
